(19) United States
(12) Patent Application Publication
(10) Pub. No.: US 2021/0161150 A1
(43) Pub. Date: Jun. 3, 2021

(54) METHODS AND COMPOSITIONS FOR KILLING A TARGET BACTERIUM

(71) Applicants: Locus Biosciences, Inc., Morrisville, NC (US); North Carolina State University, Raleigh, NC (US)

(72) Inventors: Paul M. GAROFOLO, Cary, NC (US); David G. OUSTEROUT, Raleigh, NC (US); Kurt SELLE, Raleigh, NC (US); Sandi WONG, South San Francisco, CA (US); Chase Lawrence BEISEL, Würzburg (DE); Atul Kumar SINGH, Raleigh, NC (US)

(21) Appl. No.: 17/057,601

(22) PCT Filed: May 24, 2019
(86) PCT No.: PCT/US19/34061

§ 371 Date: Nov. 20, 2020

Related U.S. Application Data
(60) Provisional application No. 62/676,818, filed on May 25, 2018.

(51) Int. Cl. A01N 63/40; C12N 15/70; C12N 7/00; A61K 35/76; C12N 15/74

(52) U.S. Cl. CPC A01N 63/40; C12N 15/70; C12N 7/00; A61K 35/76; C12N 2795/14033; C12N 2800/80; C12N 2310/20; C12N 2795/14022; C12N 15/74

(57) ABSTRACT

Provided herein are methods and compositions for killing a target bacterium with a CRISPR-Cpf1 system. Also disclosed are engineered bacteriophages.

### CROSS REFERENCE

This application claims the benefit of U.S. Provisional Application No. 62/676,818 filed May 25, 2018, which is incorporated herein by reference in its entirety.

### STATEMENT AS TO FEDERALLY SPONSORED RESEARCH

This invention was made with the support of the United States government under Grant number GM119561 awarded by the National Institutes of Health (NIH). The government has certain rights in the invention.

### SUMMARY

Disclosed herein, in certain embodiments, are methods for killing a target bacterium. In some embodiments, disclosed herein, are methods for killing a target bacterium comprises, introducing into a target bacterium a bacteriophage comprising: (a) a first nucleic acid encoding a spacer sequence or a crRNA transcribed therefrom, wherein the spacer sequence is complimentary to a target nucleotide sequence from a target gene in the target bacterium; and (b) a second nucleic acid encoding an exogenous Cpf1; wherein the target bacterium is killed by lytic activity of the bacteriophage or activity of a CRISPR-Cpf1 system using the spacer sequence or the crRNA transcribed therefrom and the exogenous Cpf1. In some embodiments, a nucleic acid encoding a transcriptional activator for a CRISPR-Cpf1 system is not introduced into a target bacterium. In other embodiments, disclosed herein, are methods for killing a target bacterium comprises, introducing into a target bacterium a bacteriophage comprising: (a) a first nucleic acid encoding a spacer sequence or a crRNA transcribed therefrom, wherein the spacer sequence is complimentary to a target nucleotide sequence from a target gene in the target bacterium; and (b) a second nucleic acid encoding a transcriptional activator for a CRISPR-Cpf1 system in a target bacterium; wherein the target bacterium is killed by lytic activity of the bacteriophage or activity of a CRISPR-Cpf1 system using the spacer sequence or the crRNA transcribed therefrom. In some embodiments, the first nucleic acid sequence is a CRISPR array further comprising at least one repeat sequence. In some embodiments, the transcriptional activator is endogenous to the target bacterium. In some embodiments, the transcriptional activator is exogenous to the target bacterium. In some embodiments, the transcriptional activator is regulated by Quorum Sensing (QS) signals. In some embodiments, the transcriptional activator is a protein involved in sensing stress of a bacterium membrane. In some embodiments, the transcriptional activator is a protein that stabilizes Cpf1. In some embodiments, the transcriptional activator is a metabolic sensing protein. In some embodiments, the metabolic sensing protein is a sigma factor. In some embodiments, the transcriptional activator disrupts the activity of an inhibitory element. In some embodiments, the inhibitory element is a transcriptional repressor. In some embodiments, the transcriptional repressor is a global transcriptional repressor. In some embodiments, the CRISPR-Cpf1 system is endogenous to the target bacterium. In some embodiments, the CRISPR-Cpf1 system is exogenous to the target bacterium. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence is at least a portion of an essential gene that is needed for the survival of the target bacterium. In some embodiments, the essential gene is yfaP, speA, ftsZ, Tsf, acpP, gapA, infA, secY, csrA, trmD, ftsA, fusA, glyQ, eno, or nusG. In some embodiments, the at least one repeat sequence is operably linked to the at least one spacer sequence at either its 5′ end or its 3′ end. In some embodiments, the target bacterium is killed solely by the lytic activity of the bacteriophage. In some embodiments, the target bacterium is killed solely by the activity of the CRISPR-Cpf1 system. In some embodiments, the target bacterium is killed by both the lytic activity of the bacteriophage and the activity of the CRISPR-Cpf1 system in combination. In some embodiments, the target bacterium is killed by the activity of the CRISPR-Cpf1 system independently of the lytic activity of the bacteriophage. In some embodiments, the activity of the CRISPR-Cpf1 system supplements or enhances the lytic activity of the bacteriophage. In some embodiments, the spacer nucleotide sequence overlaps with a second spacer sequence. In some embodiments, the lytic activity of the bacteriophage and the activity of the CRISPR-Cpf1 system are synergistic. In some embodiments, the lytic activity of the bacteriophage, the activity of the CRISPR-Cpf1 system, or both is modulated by a concentration of the bacteriophage. In some embodiments, the bacteriophage infects multiple bacterial strains. In some embodiments, the bacteriophage is an obligate lytic bacteriophage. In some embodiments, the bacteriophage is a temperate bacteriophage that is rendered lytic. In some embodiments, the bacteriophage does not confer any new properties onto the target bacterium beyond cellular death caused by the lytic activity of the bacteriophage and/or the activity of the CRISPR-Cpf1 array. In some embodiments, the target bacterium is Escherichia coli, Klebsiella pneumoniae, Salmonella enterica, or Shigella dysenteriae. In some embodiments, the first nucleic acid encoding a spacer sequence or a crRNA is inserted into a non-essential bacteriophage gene. In some embodiments, the non-essential bacteriophage gene is gp49. In some embodiments, the non-essential bacteriophage gene is gp75. In some embodiments, the non-essential bacteriophage gene is hoc. In some embodiments, the non-essential bacteriophage gene is gp0.7, gp4.3, gp4.5, or gp4.7. In some embodiments, the non-essential bacteriophage gene is gp0.6, gp0.65, gp0.7, gp4.3, or gp4.5. In some embodiments, the bacteriophage further comprises a third nucleic acid encoding a Gam protein.

Disclosed herein, in certain embodiments, are methods for modulating the activity of a CRISPR-Cpf1 system. In some embodiments, a method for modulating the activity of a CRISPR-Cpf1 system in a bacterium, comprises: introducing a bacteriophage comprising a nucleic acid encoding an exogenous Cpf1 in the target bacterium. In some embodiments, a nucleic acid encoding a transcriptional activator for a CRISPR-Cpf1 system is not introduced into a target bacterium. In some embodiments, a method for modulating the activity of a CRISPR-Cpf1 system in a bacterium, comprises: introducing a bacteriophage comprising a nucleic acid encoding a transcriptional activator for the CRISPR-Cpf1 system in the target bacterium. In some embodiments, the transcriptional activator is regulated by Quorum Sensing (QS) signals. In some embodiments, the transcriptional activator is a protein involved in sensing stress to a bacterium membrane. In some embodiments, the transcriptional activator is a protein that stabilizes Cpf1. In some embodiments, the transcriptional activator is a metabolic sensing protein. In some embodiments, the metabolic sensing protein is a sigma factor. In some embodiments, the transcriptional activator disrupts the activity of an inhibitory element. In some embodiments, the inhibitory element is a transcriptional repressor. In some embodiments, the transcriptional repressor is a global transcriptional repressor. In some embodiments, the CRISPR-Cpf1 system is endogenous to the target bacterium. In some embodiments, the CRISPR-Cpf1 system is exogenous to the target bacterium. In some embodiments, the bacteriophage infects multiple bacterial strains. In some embodiments, the bacteriophage is an obligate lytic bacteriophage. In some embodiments, the bacteriophage is a temperate bacteriophage that is rendered lytic. In some embodiments, the target bacterium is Escherichia coli, Klebsiella pneumoniae, Salmonella enterica, or Shigella dysenteriae. In some embodiments, the nucleic acid encoding a transcriptional activator is inserted into a non-essential bacteriophage gene. In some embodiments, the non-essential bacteriophage gene is gp49. In some embodiments, the non-essential bacteriophage gene is gp75. In some embodiments, the non-essential bacteriophage gene is hoc. In some embodiments, the non-essential bacteriophage gene is gp0.7, gp4.3, gp4.5, or gp4.7. In some embodiments, the non-essential bacteriophage gene is gp0.6, gp0.65, gp0.7, gp4.3, or gp4.5. In some embodiments, the bacteriophage further comprises a second nucleic acid encoding a Gam protein.

Disclosed herein, in certain embodiments, are methods for killing a target bacterium. In some embodiments, a method of killing a target bacterium comprises introducing into a target bacterium a bacteriophage comprising: (a) lytic activity, and (b) a first nucleic acid sequence encoding an anti-CRISPR polypeptide, wherein the anti-CRISPR polypeptide enhances the lytic activity of the bacteriophage. In some embodiments, the anti-CRISPR polypeptide inactivates a CRISPR-Cpf1 system. In some embodiments, the anti-CRISPR polypeptide inactivates the CRISPR-Cpf1 system using a process comprising gene regulation interference. In some embodiments, the anti-CRISPR polypeptide inactivates the CRISPR-Cpf1 system using a process comprising nuclease recruitment interference. In some embodiments, the anti-CRISPR polypeptide is a truncated protein, a fusion protein, a dimer protein or mutated protein. In some embodiments, the bacteriophage further comprises a second nucleic acid encoding a CRISPR array. In some embodiments, the CRISPR array comprises at least one repeat sequence and at least one spacer sequence that is complimentary to a target nucleotide sequence from a target gene in the target bacterium.

Disclosed herein, in certain embodiments, are bacteriophages. In some embodiments, a bacteriophage comprises: (a) a first nucleic acid encoding a spacer sequence or a crRNA transcribed therefrom, wherein the spacer sequence is complimentary to a target nucleotide sequence from a target gene in a target bacterium; and (b) a second nucleic acid encoding an exogenous Cpf1 in a target bacterium, wherein the target bacterium is killed by the lytic activity of the bacteriophage or activity of a CRISPR-Cpf1 system using the spacer sequence or the crRNA transcribed therefrom and the exogenous Cpf1. In some embodiments, a nucleic acid encoding a transcriptional activator for a CRISPR-Cpf1 system is not introduced into a target bacterium. In other embodiments, a bacteriophage comprises: (a) a first nucleic acid encoding a spacer sequence or a crRNA transcribed therefrom, wherein the spacer sequence is complimentary to a target nucleotide sequence from a target gene in a target bacterium; and (b) a second nucleic acid encoding a encoding a transcriptional activator for a CRISPR-Cpf1 system in a target bacterium, wherein the target bacterium is killed by the lytic activity of the bacteriophage or activity of a CRISPR-Cpf1 system using the spacer sequence or the crRNA transcribed therefrom. In some embodiments, the transcriptional activator is regulated by Quorum Sensing (QS) signals. In some embodiments, the transcriptional activator is a protein involved in sensing stress of a bacterium membrane. In some embodiments, the transcriptional activator is a protein that stabilizes Cpf1. In some embodiments, the transcriptional activator is a metabolic sensing protein. In some embodiments, the metabolic sensing protein is a sigma factor. In some embodiments, the transcriptional activator disrupts the activity of an inhibitory element of the target bacterium. In some embodiments, the inhibitory element is a transcriptional repressor. In some embodiments, the transcriptional repressor is a global transcriptional repressor. In some embodiments, the CRISPR-Cpf1 system is endogenous to the target bacterium. In some embodiments, the CRISPR-Cpf1 system is exogenous to the target bacterium. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence is essential. In some embodiments, the essential gene is yfaP, speA, ftsZ, Tsf, acpP, gapA, infA, secY, csrA, trmD, ftsA, fusA, glyQ, eno, or nusG. In some embodiments, the target nucleotide sequence is a non-essential gene. In some embodiments, the first nucleic acid sequence is a CRISPR array comprising at least one repeat sequence. In some embodiments, the at least one repeat sequence is operably linked to the spacer sequence at either its 5′ end or its 3′ end. In some embodiments, the bacteriophage infects multiple bacterial strains. In some embodiments, the bacteriophage is an obligate lytic bacteriophage. In some embodiments, the bacteriophage is a temperate bacteriophage that is rendered lytic. In some embodiments, the temperate bacteriophage is rendered lytic by the removal, replacement, or inactivation of one or more lysogeny genes. In some embodiments, the target bacterium is Escherichia coli, Klebsiella pneumoniae, Salmonella enterica, or Shigella dysenteriae. In some embodiments, the first nucleic acid encoding a spacer sequence or a crRNA is inserted into a non-essential bacteriophage gene. In some embodiments, the non-essential bacteriophage gene is gp49. In some embodiments, the non-essential bacteriophage gene is gp75. In some embodiments, the non-essential bacteriophage gene is hoc. In some embodiments, the non-essential bacteriophage gene is gp0.7, gp4.3, gp4.5, or gp4.7. In some embodiments, the non-essential bacteriophage gene is gp0.6, gp0.65, gp0.7, gp4.3, or gp4.5. In some embodiments, the bacteriophage further comprises a third nucleic acid encoding a Gam protein. In some embodiments, a method of treating a disease in a subject comprises administering the bacteriophage. In some embodiments, the subject is a mammal. In some embodiments, the disease is a bacterial infection. In some embodiments, a bacterium causing the bacterial infection is a bacterium in the genus Acinetobacter, Actinomyces, Burkholderia, Capylobacter, Candidia, Clostrium, Corynebacterium, Coccidiodes, Cryptococcus, Enterococcus, Escherichica, Haemophilis, Helicobacter, Klebsiella, Moraxella, Mycobacterium, Neisseria, Porphyromonas, Prevotella, Pseudomonas, Salmonella, Serratia, Staphylococcus, Streptococcus, or Vibrio. In some embodiments, a bacterium causing the bacterial infection is Burkholderia cepacia, Clostridium difficile, Corynebacterium minutissium, Corynebacterium pseudodiphtherias, Corynebacterium stratium, Escherichia coli, Haemophilus influenzae, Klebsiella pneumoniae, a Moraxella species, Mycobacterium tuberculosis, Neisseria gonorrhoeae, Neisseria meningitidis, Prevotella melaninogenicus, Salmonella typhimurium, Salmonella enterica, Shigella dysenteriae, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, Helicobacter felis, Helicobacter pylori, or Clostridium bolteae. In some embodiments, the bacterium is a drug resistant bacteria that is resistant to at least one antibiotic. In some embodiments, the bacterium is a multi-drug resistant bacteria that is resistant to at least one antibiotic. In some embodiments, the antibiotic comprises a cephalosporin, a fluoroquinolone, a carbapenem, a colistin, an aminoglycoside, vancomycin, streptomycin, or methicillin. In some embodiments, administering is intra-arterial, intravenous, intramuscular, oral, subcutaneous, inhalation, or any combination thereof. In some embodiments, pharmaceutical composition comprises the bacteriophage and a pharmaceutically acceptable excipient. In some embodiments, the pharmaceutical composition is in a form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof.

Disclosed herein, in certain embodiments, are bacteriophages comprising a nucleic acid encoding an exogenous Cpf1 in a target bacterium. In some embodiments, a nucleic acid encoding a transcriptional activator for a CRISPR-Cpf1 system is not introduced into a target bacterium. Disclosed herein, in certain embodiments, are bacteriophages comprising a nucleic acid encoding a transcriptional activator for a CRISPR-Cpf1 system in a target bacterium. In some embodiments, the transcriptional activator is regulated by Quorum Sensing (QS) signals. In some embodiments, the transcriptional activator is a protein involved in sensing stress to a bacterium membrane. In some embodiments, the transcriptional activator is a protein that stabilizes Cpf1. In some embodiments, the transcriptional activator is a metabolic sensing protein. In some embodiments, the metabolic sensing protein is a sigma factor. In some embodiments, the transcriptional activator disrupts the activity of an inhibitory element. In some embodiments, the inhibitory element is a transcriptional repressor. In some embodiments, the transcriptional repressor is a global transcriptional repressor. In some embodiments, the CRISPR-Cpf1 system is endogenous to the target bacterium. In some embodiments, the CRISPR-Cpf1 system is exogenous to the target bacterium. In some embodiments, the bacteriophage infects multiple bacterial strains. In some embodiments, the bacteriophage is an obligate lytic bacteriophage. In some embodiments, the bacteriophage is a temperate bacteriophage that is rendered lytic. In some embodiments, the target bacterium is Escherichia coli, Klebsiella pneumoniae, Salmonella enterica, or Shigella dysenteriae. In some embodiments, the nucleic acid encoding a transcriptional activator is inserted into a non-essential bacteriophage gene. In some embodiments, the non-essential gene is gp49. In some embodiments, the non-essential gene is gp75. In some embodiments, the non-essential gene is hoc. In some embodiments, the non-essential gene is gp0.7, gp4.3, gp4.5, or gp4.7. In some embodiments, the non-essential gene is gp0.6, gp0.65, gp0.7, gp4.3, or gp4.5. In some embodiments, the bacteriophage further comprises a second nucleic acid encoding a Gam protein. In some embodiments, a method of treating a disease in a subject comprises administering the bacteriophage. In some embodiments, the subject is a mammal. In some embodiments, the disease is a bacterial infection. In some embodiments, a bacterium causing the bacterial infection is a bacterium in the genus Acinetobacter, Actinomyces, Burkholderia, Capylobacter, Candidia, Clostrium, Corynebacterium, Coccidiodes, Cryptococcus, Enterococcus, Escherichica, Haemophilis, Helicobacter, Klebsiella, Moraxella, Mycobacterium, Neisseria, Porphyromonas, Prevotella, Pseudomonas, Salmonella, Serratia, Staphylococcus, Streptococcus, or Vibrio. In some embodiments, a bacterium causing the bacterial infection is Burkholderia cepacia, Clostridium difficile, Corynebacterium minutissium, Corynebacterium pseudodiphtheriae, Corynebacterium stratium, Escherichia coli, Haemophilus influenzae, Klebsiella pneumoniae, a Moraxella species, Mycobacterium tuberculosis, Neisseria gonorrhoeae, Neisseria meningitidis, Prevotella melaninogenicus, Salmonella typhimurium, Salmonella enterica, Shigella dysenteriae, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, Helicobacter felis, Helicobacter pylori, or Clostridium bolteae. In some embodiments, the bacterium is a drug resistant bacteria that is resistant to at least one antibiotic. In some embodiments, the bacterium is a multi-drug resistant bacteria that is resistant to at least one antibiotic. In some embodiments, the antibiotic comprises a cephalosporin, a fluoroquinolone, a carbapenem, a colistin, an aminoglycoside, vancomycin, streptomycin, or methicillin. In some embodiments, administering is intra-arterial, intravenous, intramuscular, oral, subcutaneous, inhalation, or any combination thereof. In some embodiments, pharmaceutical composition comprises the bacteriophage and a pharmaceutically acceptable excipient. In some embodiments, the pharmaceutical composition is in a form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof.

Disclosed herein, in certain embodiments, are bacteriophages. In some embodiments, a bacteriophage comprises (a) lytic activity, and (b) a first nucleic acid sequence encoding an anti-CRISPR polypeptide, wherein the anti-CRISPR polypeptide enhances the lytic activity of the bacteriophage. In some embodiments, a nucleic acid encoding a transcriptional activator for a CRISPR-Cpf1 system is not introduced into a target bacterium. In some embodiments, the anti-CRISPR polypeptide inactivates a CRISPR-Cpf1 system. In some embodiments, the anti-CRISPR polypeptide inactivates the CRISPR-Cpf1 system using a process comprising gene regulation interference. In some embodiments, the anti-CRISPR polypeptide inactivates the CRISPR-Cpf1 system using a process comprising nuclease recruitment interference. In some embodiments, the anti-CRISPR polypeptide is a truncated protein, a fusion protein, a dimer protein, or mutated protein. In some embodiments, the bacteriophage further comprises a second nucleic acid encoding a CRISPR array. In some embodiments, the CRISPR array comprises at least one repeat sequence and at least one spacer sequence that is complimentary to a target nucleotide sequence from a target gene in the target bacterium. In some embodiments, a method of treating a disease in a subject comprises administering the bacteriophage. In some embodiments, the subject is a mammal. In some embodiments, the disease is a bacterial infection. In some embodiments, a bacterium causing the bacterial infection is a bacterium in the genus Acinetobacter, Actinomyces, Burkholderia, Capylobacter, Candidia, Clostrium, Corynebacterium, Coccidiodes, Cryptococcus, Enterococcus, Escherichica, Haemophilis, Helicobacter, Klebsiella, Moraxella, Mycobacterium, Neisseria, Porphyromonas, Prevotella, Pseudomonas, Salmonella, Serratia, Staphylococcus, Streptococcus, or Vibrio. In some embodiments, a bacterium causing the bacterial infection is Burkholderia cepacia, Clostridium difficile, Corynebacterium minutissium, Corynebacterium pseudodiphtheriae, Corynebacterium stratium, Escherichia coli, Haemophilus influenzae, Klebsiella pneumoniae, a Moraxella species, Mycobacterium tuberculosis, Neisseria gonorrhoeae, Neisseria meningitidis, Prevotella melaninogenicus, Salmonella typhimurium, Salmonella enterica, Shigella dysenteriae, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, Helicobacter felis, Helicobacter pylori, or Clostridium bolteae. In some embodiments, the bacterium is a drug resistant bacteria that is resistant to at least one antibiotic. In some embodiments, the bacterium is a multi-drug resistant bacteria that is resistant to at least one antibiotic. In some embodiments, the antibiotic comprises a cephalosporin, a fluoroquinolone, a carbapenem, a colistin, an aminoglycoside, vancomycin, streptomycin, or methicillin. In some embodiments, administering is intra-arterial, intravenous, intramuscular, oral, subcutaneous, inhalation, or any combination thereof. In some embodiments, pharmaceutical composition comprises the bacteriophage and a pharmaceutically acceptable excipient. In some embodiments, the pharmaceutical composition is in a form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof.

### BRIEF DESCRIPTION OF THE DRAWINGS

The features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a workflow process for engineering a CRISPR-enhanced bacteriophage.

FIG. 2A-FIG. 2F illustrate comparisons of CRISPR-Cas systems (Cas9, Cpf1 (Cas12a) & Cas13a) mediated killing in E. coli MG1655. FIG. 2A illustrates features of Cas9 with its respective nucleic-acid target, PAM, gRNA and mechanism of attack. FIG. 2B illustrates features of Cpf1 (Cas12a) with its respective nucleic-acid target, PAM, spacer and mechanism of attack. FIG. 2C illustrates features of Cas13a with its respective nucleic-acid target, PAM, spacer and mechanism of attack. FIG. 2D illustrates a CRISPR array carrying different spacers (non-essential and essential) transformed in cells expressing Cas9. FIG. 2E illustrates a CRISPR array carrying different spacers (non-essential and essential) transformed in cells expressing Cpf1 (Cas12a). FIG. 2F illustrates a CRISPR array carrying different spacers (non-essential and essential) transformed in cells expressing Cas13a. Mean CFU numbers are reported for transformation in E. coli MG1655 wild-type cells. CFU count was compared with NT (Non target spacer).

FIG. 3A-FIG. 3E illustrate Cas13a mediated killing in E. coli strains. FIG. 3A illustrates CRISPR arrays carrying different spacers targeting tref and eamB (non-essential) speA and yfaP (essential) transformed in cells expressing Cas13a constitutively in E. coli MG1655. FIG. 3B illustrates CRISPR arrays carrying different spacers targeting tref and eamB (non-essential) speA and yfaP (essential) transformed in cells expressing Cas13a constitutively in E. coli BW25113. FIG. 3C illustrates CRISPR arrays carrying different spacers targeting tref and eamB (non-essential) speA and yfaP (essential) transformed in cells expressing Cas13a constitutively in E. coli BW25113ΔrecA. FIG. 3D illustrates CRISPR arrays carrying different spacers targeting tref and eamB (non-essential) speA and yfaP (essential) transformed in cells expressing Cas13a constitutively in E. coli O9:HS. FIG. 3E illustrates CRISPR arrays carrying different spacers targeting tref and eamB (non-essential) speA and yfaP (essential) transformed in cells expressing Cas13a constitutively in E. E2437A. CFU count is compared with NT (Non target spacer).

FIG. 4A-FIG. 4B illustrate killing by Cas13a with a multiplexed plasmid. FIG. 4A illustrates E. coli MG1655 with a multiplexing targeting plasmid harboring constitutively expressed Cas13a and transformations are carried out with spacer SP1 or SP2 for plasmid targeting where cells are spotted on LB-agar plate with suitable antibiotics. FIG. 4B illustrates E. coli MG1655 wild type strain with multiplexing targeting plasmid harboring constitutively expressing Cas13a and transformations carried out with spacer SP1 or SP2 for plasmid target. CFU count is compared with control.

FIG. 5A-FIG. 5C illustrate impact of recA mediated DNA repair on killing by Cas9, Cpf1 (Cas12a), and Cas13a in E. coli MG1655ΔrecA. FIG. 5A illustrates killing efficiency of a CRISPR array carrying different spacers (non-essential and essential) transformed in cells expressing Cas9 constitutively. FIG. 5B illustrates killing efficiency of a CRISPR array carrying different spacers (non-essential and essential) transformed in cells expressing Cpf1 (Cas12a) constitutively. FIG. 5C illustrates killing efficiency of a CRISPR array carrying different spacers (non-essential and essential) transformed in cells expressing Cas13a constitutively. (Mean CFU numbers are reported for transformation in E. coli MG1655 recA mutant cells. CFU count was compared with NT (Non target spacer).

FIG. 6A-FIG. 6L illustrate characteristics of cells surviving Cpf1 (Cas12a) independently by DNA damage repair via protein recA. FIG. 6A illustrates cell survivorship of cells transformed with a non-target spacer. FIG. 6B illustrates cell survivorship of cells transformed with a treF spacer. FIG. 6C illustrates cell survivorship of cells transformed with a yfaP spacer. Regions of the spacer along with 400 bp in the genome of surviving colonies were amplified to check genomic mutation. FIG. 6D illustrates genomic mutations in cells transformed with the non-target CRISPR array. FIG. 6E illustrates genomic mutations in cells transformed with the treF CRISPR array. FIG. 6F illustrates mutations in the RuvCII domain of Cpf1 (Cas12a) plasmids isolated from surviving cells transformed with the non-target CRISPR array. FIG. 6G illustrates mutations in the RuvCII domain of Cpf1 (Cas12a) plasmids isolated from surviving cells transformed with the treF CRISPR array. FIG. 6H illustrates killing efficiency of various spacer configurations (only repeat and spacer (treF_RS and yfaP_RS), repeat-spacer-repeat (treF and yfaP) and double spacer (treF+treF and yfaP+yfaP)), with a schematic diagram of the spacer with promoters are shown at the bottom of this figure. FIG. 6I illustrates a percent of one spacer sequence missing in a CRISPR array comprising a treF double spacer. FIG. 6J illustrates percentage of one spacer sequence missing in a CRISPR array comprising yfaP double spacer. FIG. 6K illustrates percentage of spacer sequence missing in a CRISPR array comprising a treF spacer. FIG. 6L illustrates percentage of spacer sequence missing in a CRISPR array comprising a yfaP spacer.

FIG. 7A-FIG. 7B illustrate Cpf1 (Cas12a) mediated killing with Repeat and spacer (R_S). FIG. 7A illustrates a schematic diagram of the repeat and the spacer. FIG. 7B illustrates the killing efficiency of three E. coli strains (E. coli BW25113, E. coli BW25113ΔrecA, and E. coli E24377A) harboring constitutively expressing Cpf1 (Cas12a) transformed with a spacer of treF with single repeat. CFU count was compared with NT (Non target spacer).

FIG. 8A-FIG. 8C illustrate the effect of Cpf1 (Cas12a) with RuvC catalytic residue mutation on killing efficiency. FIG. 8A illustrates the domain architecture of Cpf1 (Cas12a) with the RuvC catalytic residues highlighted. The catalytic residues D917 and D1255 were mutated. FIG. 8B illustrate t killing efficiency in an E. coli MG1655 wild type strain harboring constitutively expressed Cpf1 (Cas12a), Cas12aD917A, or Cas12aD1255A, where transformation was carried out with spacer of treF, eamB (non-essential) and yfaP (essential) gene. CFU count was compared with NT (Non target spacer). FIG. 8C illustrates killing efficiency in an E. coli MG1655 recA mutant strain harboring constitutively expressed Cpf1 (Cas12a), Cas12aD917A, or Cas12aD1255A, where transformation was carried out with spacer of treF, eamB (non-essential) and yfaP (essential) gene. CFU count was compared with NT (Non target spacer).

FIG. 9A-FIG. 9C illustrate the effect of Cas13a catalytic residue mutation on killing efficiency. FIG. 9A illustrates the domain architecture of Cas13a with the HEPN catalytic residues highlighted. The catalytic residues R597, H602, R1278, and H1283 were mutated. FIG. 9B illustrates killing efficiency in an E. coli MG1655 wild type strain with multiplexing targeting plasmid harboring constitutively expressed Cas13a, Cas13aR597A, Cas13aH602A, Cas13aR1278A, or Cas13aH1283A, where transformation was carried with spacer SP1 or SP2 for the plasmid target. CFU count was compared with NT (Non target spacer). FIG. 9C illustrates killing efficiency in an E. coli MG1655 wild type strain harboring constitutively expressed Cas13a, Cas13aR597A, Cas13aH602A, Cas13aR1278A, or Cas13aH1283A, where transformation was carried out with spacer SP1 or SP2 for the genome target. CFU count was compared with NT (Non target spacer).

FIG. 10A-FIG. 10G illustrate Cpf1 (Cas12a) mediated killing in broad host range of pathogens. FIG. 10A illustrates killing efficiency of CRISPR arrays carrying different spacers (non-essential and essential) in cells expressing Cpf1 (Cas12a) constitutively in E. coli BW25113. FIG. 10B illustrates killing efficiency of CRISPR arrays carrying different spacers (non-essential and essential) in cells expressing Cpf1 (Cas12a) constitutively in E. coli BW25113ΔrecA. FIG. 10C illustrates killing efficiency of CRISPR arrays carrying different spacers (non-essential and essential) in cells expressing Cpf1 (Cas12a) constitutively in E. coli O9:HS. FIG. 10D illustrates killing efficiency of CRISPR arrays carrying different spacers (non-essential and essential) in cells expressing Cpf1 (Cas12a) constitutively in E. coli E2437A. FIG. 10E illustrates killing efficiency of CRISPR arrays carrying different spacers (non-essential and essential) in cells expressing Cpf1 (Cas12a) constitutively in Shigella dysenteriae. FIG. 10F illustrates killing efficiency of CRISPR arrays carrying different spacers (non-essential and essential) in cells expressing Cpf1 (Cas12a) constitutively in Klebsiella pneumoniae. FIG. 10A illustrates killing efficiency of CRISPR arrays carrying different spacers (non-essential and essential) in cells expressing Cpf1 (Cas12a) constitutively in Salmonella enterica. CFU count was compared with NT (Non target spacer).

FIG. 11A-FIG. 11E illustrate enhanced killing of Salmonella enterica LT2. FIG. 11A illustrates schematic diagram showing the Mu Gam protein specifically binds to double stranded ends and block the DNA damage repair protein RecA which repairs DNA through homologous recombination (HR), thereby promoting cell death. FIG. 11B illustrates killing efficiency of a Salmonella enterica LT wild type and recA mutant strain harboring constitutively expressing Cpf1 (Cas12a), where transformation was carried with a CRISPR array having treF spacer (non-essential) ftsZ spacer (essential). CFU count was compared with NT (Non target spacer). FIG. 11C illustrates a schematic diagram of a single spacer and multiplex spacer. FIG. 11D illustrate killing efficiency of a Salmonella enterica LT wild type strain harboring constitutively expressing Cpf1 (Cas12a), where transformation was carried out with single and multiplex spacer treF. CFU count was compared with NT (Non target spacer). FIG. 11E illustrates killing efficiency of a Salmonella enterica LT2 wild type strain harboring constitutively expressing Cpf1 (Cas12a) and Gam protein, where transformation was carried out with single and multiplex spacer treF. CFU count was compared with NT (Non target spacer).

FIG. 12 illustrates upregulation of rdgC accounts for Cas13a-mediated killing of E. coli MG1655ΔrecA. mRNA levels of rdgC and soxS were analyzed by qRT-PCR in E. coli MG1655 or E. coli MG1655ΔrecA expressing the Cas13a and a single-spacer CRISPR array targeting the indicated gene. NT—non-targeting. Results were representative of three independent experiments starting from separate colonies.

FIG. 13A-FIG. 13F illustrate schematics of plasmid maps. FIG. 13A illustrates a schematic of a pBAD33-Cpf1. FIG. 13B illustrates a schematic of a pBAD33-Cpf1-MuGam. FIG. 13C illustrates a schematic of a pACYC184-Cas9. FIG. 13D illustrates a schematic of a pACYC184-Cas13a. FIG. 13E illustrates a schematic of the sgRNA plasmid for Cas9. FIG. 13F illustrates a schematic of the spacer plasmid for Cpf1 (Cas12a) and C2c2 (Cas13a).

FIG. 14A-FIG. 14B illustrates plasmid expressed Cpf1 and self-targeting crRNAs elicit cell death. FIG. 14A illustrates plasmid transformation of CpfI alone and CpfI with crRNAs targeting ftsA or gyrB and exemplifies that bacterial genome targeted by plasmid transformed with CPF1+crRNA causes bacterial cell death and further shows its utility as a nuclease for phage-delivered anti-microbial activity in two Pseudomonas aeruginosa strains. FIG. 14B illustrates comparison of phage titers for a wild-type Pseudomonas aeruginosa phage, CpfI encoding phage and CpfI+crRNA encoding phage on two Pseudomonas strains. Pseudomonas aeruginosa phage (p1032) was engineered to carry either the Cpf1 coding sequence alone or in concert with the ftsA crRNA and assessed for their ability to amplify. The results illustrate that the Cpf1 and Cpf1+crRNA variants exhibited the same fitness in terms of final titer amplification as the wild-type counterpart on two Pseudomonas aeruginosa strains.

FIG. 14C illustrates comparison of Pseudomonas aeruginosa strain cfu reductions by a wild-type Pseudomonas aeruginosa phage, cpfI encoding phage and cpfI+crRNA encoding phage. p1032 and its engineered variants were incubated with a susceptible Pseudomonas aeruginosa strain (b1127) and sampled at various times to enumerate bacterial cfus.

FIG. 15A-FIG. 15B illustrate p1106 and engineered phages CFU reduction assays for PA14. p1106 and its engineered variants were incubated with a susceptible Pseudomonas aeruginosa strain (PA14, FIG. 15A) and a non-susceptible strain (LFP1160, FIG. 15B) and sampled at various times to enumerate bacterial CFUs.

FIG. 16 is an exemplary schematic for detection of Cpf1 and crRNA expression in phage p1032.

FIG. 17A-FIG. 17D illustrate Cpf1 expression at various time points. The fold changes were derived by comparison to the uninfected control at each individual timepoint. The fold changes were compared against the Pseudomonas aeruginosa housekeeping gene, rpsH. The background expression in the WT phage-infected bacteria was minimal. Cpf1 appears to only be expressed in the crPhage, indicating the specificity of the primers for detecting CPFI expression.

FIG. 18 illustrates that Cpf1 appears to be expressed in the crPhage and expression appears to increase over time. The fold changes were derived by comparison to the uninfected control at 15 min timepoint. The fold changes were compared against the Pseudomonas aeruginosa housekeeping gene, rpsH. The background expression in the WT phage-infected bacteria was minimal.

FIG. 19A-FIG. 19D illustrate crRNA expression at various time points. The fold changes were derived by comparison to the uninfected control at each individual timepoint. The fold changes were compared against the Pseudomonas aeruginosa housekeeping gene, rpsH. The background expression in the WT phage-infected bacteria was minimal. crRNA appears to be expressed in the crPhage.

FIG. 20 illustrates that crRNA appears to be expressed in the crPhage and expression appears to increase over time. The fold changes were derived by comparison to the uninfected control at 15 min timepoint. The fold changes were compared against the Pseudomonas aeruginosa housekeeping gene, rpsH. The background expression in the WT phage-infected bacteria was minimal.

FIG. 21A-FIG. 21D illustrate phage DNA polymerase expression in WT phage and crPhage at various time points. The fold changes were derived by comparison to the uninfected control at each individual timepoint. The fold changes were compared against the Pseudomonas aeruginosa housekeeping gene, rpsH.

FIG. 22 illustrates phage DNA polymerase expression in WT phage and crPhage and exemplifies that expression increases over time. The fold changes were derived by comparison to the uninfected control at 15 min timepoint. The fold changes were compared against the Pseudomonas aeruginosa housekeeping gene, rpsH.

FIG. 23A-FIG. 23D illustrate uncut ftsA expression at various time points. The fold changes were derived by comparison to the uninfected control at each individual timepoint. The fold changes were compared against the Pseudomonas aeruginosa housekeeping gene, rpsH.

FIG. 24 illustrates uncut ftsA expression. The fold changes were derived by comparison to the uninfected control at 15 min timepoint. The fold changes were compared against the Pseudomonas aeruginosa housekeeping gene, rpsH

FIG. 25A-FIG. 25D illustrate cut ftsA expression at various time points. The fold changes were derived by comparison to the uninfected control at each individual timepoint. The fold changes were compared against the Pseudomonas aeruginosa housekeeping gene, rpsH.

FIG. 26 illustrates cut ftsA expression. The fold changes were derived by comparison to the uninfected control at 15 min timepoint. The fold changes were compared against the Pseudomonas aeruginosa housekeeping gene, rpsH.

FIG. 27 illustrates ratio of cut:uncut ftsA by fold changes. When the DNA is being cut, the ratio is less than 1.

### DETAILED DESCRIPTION

### Definitions

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

Unless the context indicates otherwise, it is specifically intended that the various features of the disclosure described herein are able of being used in any combination. Moreover, the present disclosure also contemplates that in some embodiments, any feature or combination of features set forth herein are excluded or omitted. To illustrate, if the specification states that a composition comprises components A, B and C, it is specifically intended that any of A, B or C, or a combination thereof, are omitted and disclaimed singularly or in any combination.

One of skill in the art will understand the interchangeability of terms designating the various CRISPR-Cas systems and their components due to a lack of consistency in the literature and an ongoing effort in the art to unify such terminology. Likewise, one of skill in the art will also understand the interchangeability of terms designating the various anti-CRISPR proteins due to a lack of consistency in the literature and an ongoing effort in the art to unify such terminology.

As used in the description and the appended claims, the singular forms “a,” “an” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

The term “about” as used herein when referring to a measurable value such as a dosage or time period and the like refers to variations of ±20%, ±10%, ±5%, ±1%, +0.5%, or even ±0.1% of the specified amount. As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The terms “comprise”, “comprises”, and “comprising”, “includes”, “including”, “have” and “having”, as used herein, specify the presence of the stated features, steps, operations, elements, and/or components, but do not preclude the presence or addition of one or more other features, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed disclosure. Thus, the term “consisting essentially of” when used in a claim of this disclosure is not intended to be interpreted to be equivalent to “comprising.”

The terms “consists of” and “consisting of”, as used herein, exclude any features, steps, operations, elements, and/or components not otherwise directly stated. The use of “consisting of” limits only the features, steps, operations, elements, and/or components set forth in that clause and does exclude other features, steps, operations, elements, and/or components from the claim as a whole.

As used herein, “chimeric” refers to a nucleic acid molecule or a polypeptide in which at least two components are derived from different sources (e.g., different organisms, different coding regions).

“Complement” as used herein means 100% complementarity or identity with the comparator nucleotide sequence or it means less than 100% complementarity (e.g., about 80%, 81%, 82%, 83%, 84%, 85%, 86%, 87%, 88%, 89%, 90%, 91%, 92%, 93%, 94%, 95%, 96%, 97%, 98%, 99%, and the like, complementarity). Complement or complementable may also be used in terms of a “complement” to or “complementing” a mutation.

The terms “complementary” or “complementarity”, as used herein, refer to the natural binding of polynucleotides under permissive salt and temperature conditions by base-pairing. For example, the sequence “A-G-T” binds to the complementary sequence “T-C-A.” Complementarity between two single-stranded molecules is “partial,” in which only some of the nucleotides bind, or it is complete when total complementarity exists between the single stranded molecules. The degree of complementarity between nucleic acid strands has effects on the efficiency and strength of hybridization between nucleic acid strands.

As used herein, the term “gene” refers to a nucleic acid molecule capable of being used to produce mRNA, tRNA, rRNA, miRNA, anti-microRNA, regulatory RNA, and the like. Genes may or may not be capable of being used to produce a functional protein or gene product. Genes include both coding and non-coding regions (e.g., introns, regulatory elements, promoters, enhancers, termination sequences and/or 5′ and 3′ untranslated regions). A gene is “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

As used herein, a “target nucleotide sequence” refers to the portion of a target gene that is complementary to the spacer sequence of the recombinant CRISPR array.

As used herein, a “target DNA,” “target nucleotide sequence,” “target region,” or a “target region in the genome” refers to a region of an organism's genome that is fully complementary or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a CRISPR array. In some embodiments, a target region is about 10 to about 40 consecutive nucleotides in length located immediately adjacent to a PAM (protospacer adjacent motif) sequence (PAM sequence located immediately 3′ of the target region) in the genome of the organism. In some embodiments, a target nucleotide sequence is located adjacent to or flanked by a PAM. While PAMs are often specific to the particular CRISPR-Cas system, a PAM sequence is determined by a suitable method. Thus, for example, experimental approaches include targeting a sequence flanked by all possible nucleotides sequences and identifying sequence members that do not undergo targeting, such as through in vitro cleavage of target DNA or the transformation of target plasmid DNA. In some embodiments, a computational approach includes performing BLAST searches of natural spacers to identify the original target DNA sequences in bacteriophages or plasmids and aligning these sequences to determine conserved sequences adjacent to the target sequence.

As used herein, the term “protospacer adjacent motif” or “PAM” refers to a DNA sequence present on the target DNA molecule adjacent to the sequence matching the guide RNA spacer. This motif is found in the target gene next to the region to which a spacer sequence binds as a result of being complementary to that region and identifies the point at which base pairing with the spacer nucleotide sequence begins. For type V systems, the PAM is located immediately 3′ to the sequence that matches the spacer, and thus is 5′ to the sequence that base pairs with the spacer nucleotide sequence. Non-limiting examples of a PAM includes YTN, wherein Y is a pyrimidine and N is any nucleobase. In some embodiments, for Cpf1, the PAM is TTN or TTTV.

A “CRISPR array” as used herein means a nucleic acid molecule that comprises at least two repeat sequences, or a portion of each of said repeat sequences, and at least one spacer sequence. One of the two repeat sequences, or a portion thereof, is linked to the 5′ end of the spacer sequence and the other of the two repeat sequences, or portion thereof, is linked to the 3′ end of the spacer sequence. In a recombinant CRISPR array, the combination of repeat sequences and spacer sequences is synthetic, made by man and not found in nature. In some embodiments, a “CRISPR array” refers to a nucleic acid construct that comprises from 5′ to 3′ at least one repeat-spacer sequences (e.g., about 1, 2, 3, 4, 5, 6, 7, 8, 9, 10, 11, 12, 13, 14, 15, 16, 17, 18, 19, 20, 21, 22, 23, 24, 25 or more repeat-spacer sequences, and any range or value therein), wherein the 3′ end of the 3′ most repeat-spacer sequence of the array are linked to a repeat sequence, thereby all spacers in said array are flanked on both the 5′ end and the 3′ end by a repeat sequence.

As used herein, “spacer sequence” or “spacer refers to a nucleotide sequence that is complementary to a target DNA (i.e., target region in the genome or the “protospacer sequence,” which is adjacent to a protospacer adjacent motif (PAM) sequence). The spacer sequence is fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target DNA.

A “repeat sequence” as used herein, refers to, for example, any repeat sequence of a wild-type CRISPR locus or a repeat sequence of a synthetic CRISPR array that is separated by “spacer sequences” (e.g., repeat-spacer-repeat sequences). A repeat sequence useful with this disclosure is any known or later identified repeat sequence of a CRISPR locus or it is a synthetic repeat designed to function in a CRISPR system, for example CRISPR-Cpf1. Cpf1 is also referred to herein as Cas12a.

In some embodiments, Cpf1, CRISPR-associate endonuclease Cas12a, Cas12a, CRISPR-associated endonuclease Cpf1 are used interchangeably. Cpf1 is an RNA-guided endonuclease of a class II CRISPR/Cas system that recognizes a 5′ T-rich protospacer adjacent motif, wherein double strand DNA cleavage results in a 5′ overhang. Type V systems have been identified in several bacteria, including Parcubacteria bacterium GWC2011_GWC2_44_17 (PbCpf1), Lachnospiraceae bacterium MC2017 (Lb3Cpf1), Butyrivibrio proteoclasticus (BpCpf1), Peregrinibacteria bacterium GW2011_WA_33_10 (PeCpf1), Acidaminococcus spp. BV3L6 (AsCpf1), Porphyromonas macacae (PmCpf1), Lachnospiraceae bacterium ND2006 (LbCpf1), Porphyromonas crevioricanis (PcCpf1), Prevotella disiens (PdCpf1), Moraxella bovoculi 237(MbCpf1), Smithella spp. SC_K08D17 (SsCpf1), Leptospira inadai (LiCpf1), Lachnospiraceae bacterium MA2020 (Lb2Cpf1), Franciscella novicida U112 (FnCpf1), Candidatus methanoplasma termitum (CMtCpf1), and Eubacterium eligens (EeCpf1). GenBank Accession numbers for Cpf1 are readily available, for example, Lachnospiraceae bacterium (GenBank Accession number WP_051666128.1), Acidaminococcus (GenBank Accession number WP_021736722.1), Francisella novicida (GenBank Accession number AVC43833.1), Francisella novicida (GenBank Accession number WP003034647), Francisella tularensis (GenBank Accession number WP_071304624.1). In some embodiments, Cpf1 as used herein, also refer to variants, fusions, and nucleic acid complexes related thereto.

As used herein, the term “CRISPR phage”, “CRISPR enhanced phage”, and “crPhage” refer to bacteriophage particles comprising bacteriophage DNA comprising at least one heterologous polynucleotide. In some embodiments, the polynucleotide encodes at least one component of a CRISPR-Cpf1 system (e.g., CRISPR array, crRNA; e.g., PI bacteriophage comprising an insertion of crRNA targeting). In some embodiments, the polynucleotide encodes Cpf1 of a CRISPR-Cpf1 system. In some embodiments, the polynucleotide encodes a Cpf1 crRNA. In some embodiments, a Cpf1 crRNA nucleic acid sequence is used to direct activity of exogenous Cpf1 to endogenous chromosomal sequences in bacteria to induce double strand breaks. In some embodiments, the polynucleotide encodes at least one transcriptional activator of a CRISPR-Cpf1 system. In some embodiments, the polynucleotide encodes at least one component of an anti-CRISPR polypeptide of a CRISPR-Cpf1 system.

As used herein, the phrase “substantially identical,” or “substantial identity” in the context of two nucleic acid molecules, nucleotide sequences or protein sequences, refer to two or more sequences or subsequences that have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments, substantial identity refer to two or more sequences or subsequences that have at least about 70%, at least about 75%, at least about 80%, at least about 85%, at least about 90%, at least about 95, 96, 97, 98, or 99% identity. For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences is to a full-length polynucleotide sequence or to a portion thereof, or to a longer polynucleotide sequence. In some embodiment, “percent identity” is also determined using BLASTX version 2.0 for translated nucleotide sequences and BLASTN version 2.0 for polynucleotide sequences.

In some embodiments, the recombinant nucleic acids molecules, nucleotide sequences and polypeptides disclosed herein are “isolated.” An “isolated” nucleic acid molecule, an “isolated” nucleotide sequence or an “isolated” polypeptide is a nucleic acid molecule, nucleotide sequence or polypeptide that exists apart from its native environment. In some embodiments, an isolated nucleic acid molecule, nucleotide sequence or polypeptide exist in a purified form that is at least partially separated from at least some of the other components of the naturally occurring organism or virus, for example, the cell or viral structural components or other polypeptides or nucleic acids commonly found associated with the polynucleotide. In representative embodiments, the isolated nucleic acid molecule, the isolated nucleotide sequence and/or the isolated polypeptide is at least about 1%, 5%, 10%, 20%, 30%, 40%, 50%, 60%, 70%, 80%, 90%, 95%, or more pure.

As used herein, the terms “anti-CRISPR” or “Acr” refers to any protein or gene product with functional anti-CRISPR activity. Due to a lack of consistency in the literature, one of skill in the art will understand the interchangeability of terms designating the various anti-CRISPR proteins. For example, as used herein the designation of Acr1-Bo is interchangeable with AcrIIC1Boe and the designation of Acr2-Nm is interchangeable with AcrIIC2Nme. Also, as used herein, the designation of Acr88a-32 is interchangeable with AcrE2. An anti-CRISPR protein is any bacteriophage protein with activity that prevents the function of a bacterial CRISPR-Cas system, such as a CRISPR-Cpf1 system. Activity of an anti-CRISPR protein prevents a host bacterium from mounting a CRISPR-Cas system based defense against the invading bacteriophage.

By the terms “treat,” “treating,” or “treatment,” it is intended that the severity of the subject's condition is reduced or at least partially improved or modified and that some alleviation, mitigation or decrease in at least one clinical symptom is achieved, and/or there is a delay in the progression of the disease or condition, and/or delay of the onset of a disease or illness. With respect to an infection, a disease or a condition, the term refers to a decrease in the symptoms or other manifestations of the infection, disease or condition. In some embodiments, treatment provides a reduction in symptoms or other manifestations of the infection, disease or condition by at least about 5%, e.g., about 10%, 15%, 20%, 25%, 30%, 35%, 40%, 45%, 50% or more.

The terms with respect to an “infection”, “a disease”, or “a condition”, used herein, refer to any adverse, negative, or harmful physiological condition in a subject. In some embodiments, the source of an “infection”, “a disease”, or “a condition”, is the presence of a target bacterial population in and/or a subject. In some embodiments, the bacterial population comprises one or more target bacterial species. In some embodiments, the one or more bacteria in the bacterial population comprise one or more strains of one or more bacteria. In some embodiments, the target bacterial population causing an “infection”, “a disease”, or “a condition” is acute or chronic. In some embodiments, the target bacterial population causing an “infection”, “a disease”, or “a condition” is localized or systemic. In some embodiments, the target bacterial population causing an “infection”, “a disease”, or “a condition” is idiopathic. In some embodiments, the target bacterial population causing an “infection”, “a disease”, or “a condition” is acquired through means, including but not limited to, respiratory inhalation, ingestion, skin and wound infections, blood stream infections, middle-ear infections, gastrointestinal tract infections, peritoneal membrane infections, urinary tract infections, urogenital tract infections, oral soft tissue infections, intra-abdominal infections, epidermal or mucosal absorption, eye infections (including contact lens contamination), endocarditis, infections in cystic fibrosis, infections of indwelling medical devices such as joint prostheses, dental implants, catheters and cardiac implants, sexual contact, and/or hospital-acquired and ventilator-associated bacterial pneumonias.

As used herein the term “biofilm” means an accumulation of microorganisms embedded in a matrix of polysaccharide. In some embodiments, biofilms form on solid biological or non-biological surfaces and are medically important, accounting for over 80 percent of microbial infections in the body.

The terms “prevent,” “preventing,” and “prevention” (and grammatical variations thereof) refer to prevention and/or delay of the onset of an infection, disease, condition and/or a clinical symptom(s) in a subject and/or a reduction in the severity of the onset of the infection, disease, condition and/or clinical symptom(s) relative to what occurs in the absence of carrying out the methods disclosed herein prior to the onset of the disease, disorder and/or clinical symptom(s). Thus, in some embodiments, to prevent infection, food, surfaces, medical tools and devices are treated with compositions and by methods disclosed herein.

A “subject” disclosed herein includes any animal that has or is susceptible to an infection, disease or condition involving bacteria. Thus, in some embodiments, subjects are mammals, avians, reptiles, amphibians, or fish. Mammalian subjects include but are not limited to humans, non-human primates (e.g., gorilla, monkey, baboon, and chimpanzee, etc.), dogs, cats, goats, horses, pigs, cattle, sheep, and the like, and laboratory animals (e.g., rats, guinea pigs, mice, gerbils, hamsters, and the like). Avian subjects include but are not limited to chickens, ducks, turkeys, geese, quail, pheasants, and birds kept as pets (e.g., parakeets, parrots, macaws, cockatoos, canaries, and the like). In some embodiments, suitable subjects include both males and females and subjects of any age, including embryonic (e.g., in-utero or in-ovo), infant, juvenile, adolescent, adult and geriatric subjects. In some embodiments, a subject is a human.

By “pharmaceutically acceptable” it is meant a material that is not biologically or otherwise undesirable, i.e., the material are administered to a subject without causing any undesirable biological effects such as toxicity.

As discussed in more detail herein, including in the summary of invention herein, provided herein are methods of killing bacterium (e.g., target bacterium, such as with bacteriophages suited for, designed for, or suitable for killing such target bacterium, such as selectively killing such target bacterium), methods of modulating CRISPR-Cpf1 systems, bacteriophages (e.g., suited for, designed for, or suitable for killing such target bacterium, such as selectively killing such target bacterium), and other components, steps, and other parts either individually or in combination as described in the summary or otherwise herein.

Specifically, disclosed in certain embodiments herein, are methods for killing a target bacterium. In some embodiments, disclosed herein, are methods for killing a target bacterium. In some embodiments, the method comprises, introducing into a target bacterium a bacteriophage comprising: (a) a first nucleic acid encoding a spacer sequence or a crRNA transcribed therefrom, and (b) a second nucleic acid encoding an exogenous Cpf1. In some embodiments, the method comprises, introducing into a target bacterium a bacteriophage comprising: (a) a first nucleic acid encoding a spacer sequence or a crRNA transcribed therefrom, and (b) a second nucleic acid encoding a transcriptional activator for a CRISPR-Cpf1 system in a target bacterium. In specific embodiments, the spacer sequence is complimentary to a target nucleotide sequence from a target gene in the target bacterium. In some specific embodiments, the target bacterium is killed by lytic activity of the bacteriophage and/or activity of a CRISPR-Cpf1 system using the spacer sequence or the crRNA transcribed therefrom.

In certain embodiments, provided herein are methods for modulating the activity of a CRISPR-Cpf1 system. In some embodiments, a method for modulating the activity of a CRISPR-Cpf1 system in a bacterium, comprises: introducing a bacteriophage comprising a nucleic acid encoding an exogenous Cpf1. In some embodiments, a method for modulating the activity of a CRISPR-Cpf1 system in a bacterium, comprises: introducing a bacteriophage comprising a nucleic acid encoding a transcriptional activator for the CRISPR-Cpf1 system in the target bacterium.

In certain embodiments, provided herein are methods for killing a target bacterium. In some embodiments, a method of killing a target bacterium comprises introducing into a target bacterium a bacteriophage comprising: (a) lytic activity, and (b) a first nucleic acid sequence encoding an anti-CRISPR polypeptide, wherein the anti-CRISPR polypeptide enhances the lytic activity of the bacteriophage. In some embodiments, the anti-CRISPR polypeptide inactivates a CRISPR-Cpf1 system. In some embodiments, the anti-CRISPR polypeptide inactivates the CRISPR-Cpf1 system using a process comprising gene regulation interference. In some embodiments, the anti-CRISPR polypeptide inactivates the CRISPR-Cpf1 system using a process comprising nuclease recruitment interference.

In certain embodiments, provided herein are bacteriophages. In some embodiments, a bacteriophage comprises a first nucleic acid encoding a spacer sequence or a crRNA transcribed therefrom. In specific embodiments, the spacer sequence is complimentary to a target nucleotide sequence from a target gene in a target bacterium. In some embodiments, the bacteriophage comprises a second nucleic acid encoding a encoding an exogenous Cpf1. In some embodiments, the bacteriophage comprises a second nucleic acid encoding a encoding a transcriptional activator for a CRISPR-Cpf1 system in a target bacterium. In specific embodiments, the target bacterium is killed by the lytic activity of the bacteriophage or activity of a CRISPR-Cpf1 system using the spacer sequence or the crRNA transcribed therefrom.

In certain embodiments, are bacteriophages comprising a nucleic acid encoding an exogenous Cpf1.

In certain embodiments, are bacteriophages comprising a nucleic acid encoding a transcriptional activator for a CRISPR-Cpf1 system in a target bacterium.

In certain embodiments, disclosed herein are bacteriophages. In some embodiments, a bacteriophage comprises (a) lytic activity, and (b) a first nucleic acid sequence encoding an anti-CRISPR polypeptide. In specific embodiments, the anti-CRISPR polypeptide enhances the lytic activity of the bacteriophage. In some embodiments, the anti-CRISPR polypeptide inactivates a CRISPR-Cpf1 system. In some embodiments, the anti-CRISPR polypeptide inactivates the CRISPR-Cpf1 system using a process comprising gene regulation interference. In some embodiments, the anti-CRISPR polypeptide inactivates the CRISPR-Cpf1 system using a process comprising nuclease recruitment interference.

In certain embodiments, a bacteriophage provided herein selectively kills a target bacteria or bacterium, e.g., such that the bacteria that is not the target bacterium or bacteria is killed at a lesser rate than the target bacteria, such as at less than 50% the rate, less than 25% the rate, less than 10% the rate, or about 0% the rate (i.e., not at all) relative to the target bacterium or bacteria. In some instances, such as in certain methods provided herein, less than 50% of the non-target bacterium is killed, less than 25%, less than 20%, less than 10%, less than 5% killed, or the like is killed.

### CRISPR/Cpf1 Systems

CRISPR-Cpf1 systems are naturally adaptive immune systems found in bacteria and archaea. The CRISPR system is a nuclease system involved in defense against invading phages and plasmids that provides a form of acquired immunity.

In some embodiments, processing of a CRISPR-array disclosed herein includes, but is not limited to, the following processes: 1) transcription of the nucleic acid encoding a CRISPR array into a pre-crRNA; 2) pre-crRNA processing by Cpf1 into mature crRNAs; 3) mature crRNA complexation with Cpf1; 4) target recognition by the complexed mature crRNA/Cpf1; and 5) nuclease activity at the target leading to double stranded DNA breakage resulting in a 5′ overhang.

In some embodiments, a CRISPR array disclosed herein comprises a nucleic acid that encodes a processed, mature crRNA. In some embodiments, a mature crRNA is introduced into a phage or a target bacterium described herein. In some embodiments, a phage comprises a nucleic acid that encodes a processed, mature crRNA. In some embodiments, an endogenous or exogenous Cpf1 processes a CRISPR array into mature crRNA. In some embodiments, an exogenous Cpf1 is introduced into a phage. In some embodiments, a phage comprises an exogenous Cpf1. In some embodiments, an exogenous Cpf1 is introduced into a target bacterium.

In some embodiments, the CRISPR-Cpf1 system is endogenous to the target bacterium. In some embodiments, the CRISPR-Cpf1 system is exogenous to the target bacterium.

### CRISPR Array

Disclosed herein are CRISPR arrays compatible with Cpf1. In some embodiments, a nucleic acid encoding a CRISPR array comprises at least one repeat sequence and at least one spacer sequence complimentary to a target nucleotide sequence from a target gene in the target bacterium. In some embodiments, a CRISPR array is of any length and comprises any number of spacer nucleotide sequences alternating with repeat nucleotide sequences necessary to achieve the desired level of killing of the target bacterium by use of one or more target genes. In some embodiments, the CRISPR array comprise, consist essentially of, or consist of 1 to about 100 spacer nucleotide sequences, each linked on its 5′ end and its 3′ end to a repeat nucleotide sequence. In some embodiments, a recombinant CRISPR array of disclosed herein, consist essentially of, or consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer nucleotide sequences.

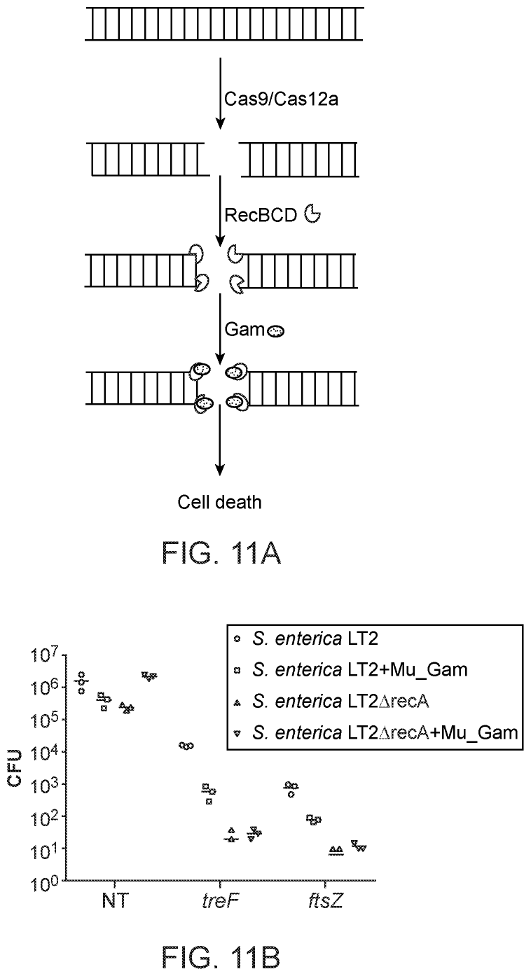

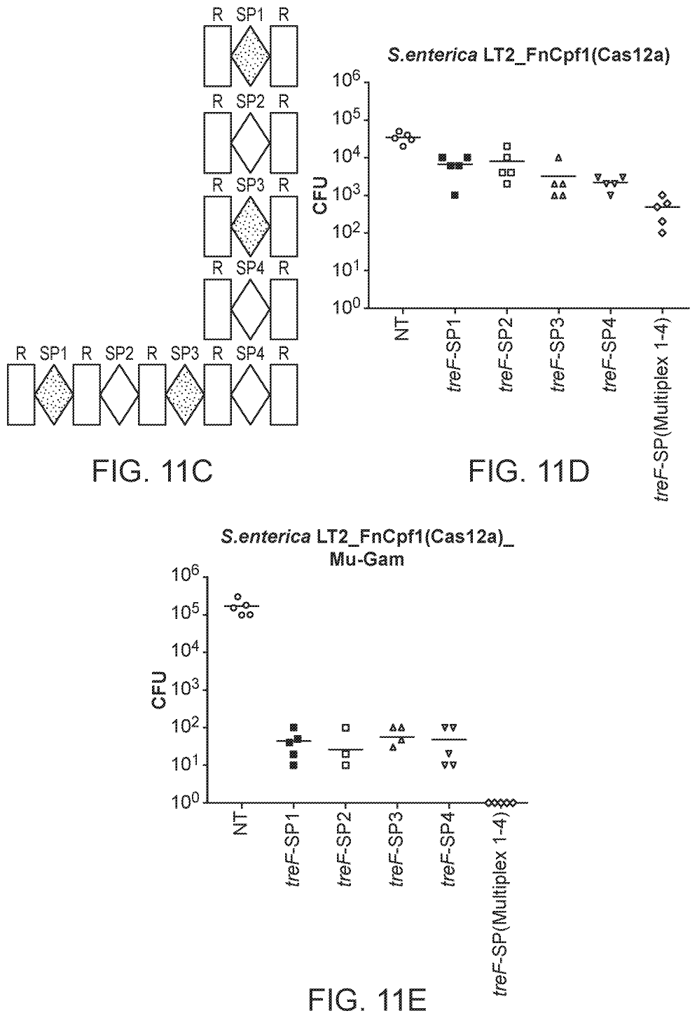

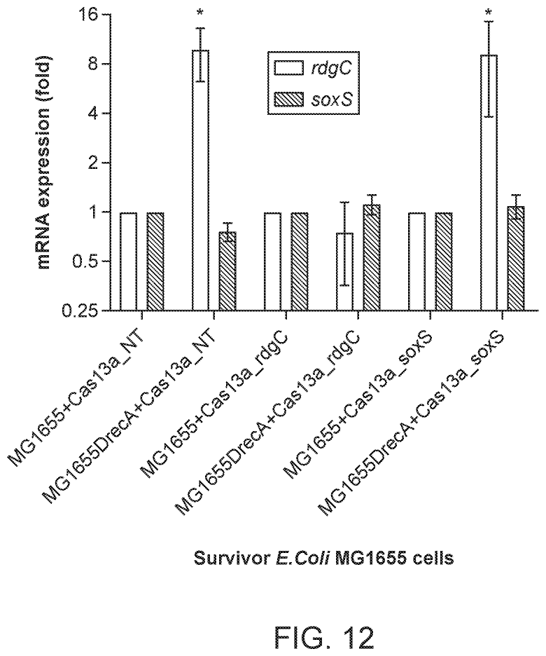

In some embodiments, the CRISPR array comprises a plurality of spacers, wherein each spacer targets a plurality of genomic locations of the target gene, herein referred to as a multiplex spacer). In some embodiments, a multiple spacer comprises at least two, at least three, at least four, or at least five spacers. In some embodiments, a multiple spacer comprises at least four spacers. An example of a multiplex spacer comprising four spacers compared to four single spacers is illustrated in FIG. 11C. Described herein, in certain embodiments, are methods for killing a target bacterium comprising administering a vector comprising a nucleic acid encoding a multiplex spacer sequence or a crRNA transcribed therefrom, wherein the multiplex spacer sequence comprises at least two spacers complimentary to at least two target nucleotide sequences from a target gene in the target bacterium. In some embodiments, a multiplex spacer sequence comprises spacers targeting essential genes. In some embodiments, a multiplex spacer sequence comprises spacers targeting only essential genes. In some embodiments, a multiplex spacer sequence comprises spacers targeting non-essential genes. In some embodiments, a multiplex spacer sequence comprises spacers targeting only non-essential genes. In some embodiments, a multiplex spacer sequence comprises spacers targeting essential genes and non-essential genes. In some embodiments, a multiplex spacer is a length described herein.

Spacer

In some embodiments, the spacer sequence described herein comprises one, two, three, four, or five mismatches as compared to the target DNA. In some embodiments, mismatches are contiguous. In some embodiments, mismatches are noncontiguous. In some embodiments, the spacer sequence has 70% complementarity to a target DNA. In some embodiments, the spacer nucleotide sequence has 80% complementarity to a target DNA. In some embodiments, the spacer nucleotide sequence is 85%, 90%, 95%, 96%, 97%, 98%, 99% or 100% complementarity to a target nucleotide sequence of a target gene. In some embodiments, the spacer sequence has 100% complementarity to the target DNA. In some embodiments, a spacer sequence has complete complementarity or substantial complementarity over a region of a target nucleotide sequence that are at least about 8 nucleotides to about 150 nucleotides in length. In some embodiments, a spacer sequence has complete complementarity or substantial complementarity over a region of a target nucleotide sequence that is at least about 20 nucleotides to about 100 nucleotides in length. In some embodiments, the 5′ region of a spacer sequence is 100% complementary to a target DNA while the 3′ region of the spacer is substantially complementary to the target DNA and therefore the overall complementarity of the spacer sequence to the target DNA is less than 100%. For example, in some embodiments, the first 7, 8, 9, 10, 11, 12, 13, 14, 15, 16 nucleotides in the 3′ region of a 20 nucleotide spacer sequence (seed region) is 100% complementary to the target DNA, while the remaining nucleotides in the 5′ region of the spacer sequence are substantially complementary (e.g., at least about 70% complementary) to the target DNA. In some embodiments, the first 7 to 12 nucleotides of the 3′ end of the spacer sequence is 100% complementary to the target DNA, while the remaining nucleotides in the 5′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target DNA. In some embodiments, the first 7 to 10 nucleotides in the 3′ end of the spacer sequence is 75%-99% complementary to the target DNA, while the remaining nucleotides in the 5′ region of the spacer sequence are at least about 50% to about 99% complementary to the target DNA. In some embodiments, the first 7 to 10 nucleotides in the 3′ end of the spacer sequence are 100% complementary to the target DNA, while the remaining nucleotides in the 5′ region of the spacer sequence are substantially complementary (e.g., at least about 70% complementary) to the target DNA. In some embodiments, the first 10 nucleotides (within the seed region) of the spacer sequence are 100% complementary to the target DNA, while the remaining nucleotides in the 5′ region of the spacer sequence are substantially complementary (e.g., at least about 70% complementary) to the target DNA. In some embodiment, the 5′ region of a spacer sequence (e.g., the first 8 nucleotides at the 5′ end, the first 10 nucleotides at the 5′ end, the first 15 nucleotides at the 5′ end, the first 20 nucleotides at the 5′ end) have about 75% complementarity or more (75% to about 100% complementarity) to a target DNA, while the remainder of the spacer sequence have about 50% or more complementarity to the target DNA. In some embodiments, the first 8 nucleotides at the 5′ end of a spacer sequence have 100% complementarity to the target nucleotide sequence or have one or two mutations and therefore are about 88% complementary or about 75% complementary to a target DNA, respectively, while the remainder of the spacer nucleotide sequence is at least about 50% or more complementary to the target DNA.

In some embodiments, a spacer sequence described herein is about 15 nucleotides to about 150 nucleotides in length. In some embodiments, a spacer nucleotide sequence is about 15 nucleotides to about 100 nucleotides in length (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nucleotides or more). In some embodiments, a spacer nucleotide sequence is a length of about 8 to about 150 nucleotides, about 8 to about 100 nucleotides, about 8 to about 50 nucleotides, about 8 to about 40 nucleotides, about 8 to about 30 nucleotides, about 8 to about 25 nucleotides, about 8 to about 20 nucleotides, about 10 to about 150 nucleotides, about 10 to about 100 nucleotides, about 10 to about 80 nucleotides, about 10 to about 50 nucleotides, about 10 to about 40, about 10 to about 30, about 10 to about 25, about 10 to about 20, about 15 to about 150, about 15 to about 100, about 15 to about 50, about 15 to about 40, about 15 to about 30, about 20 to about 150 nucleotides, about 20 to about 100 nucleotides, about 20 to about 80 nucleotides, about 20 to about 50 nucleotides, about 20 to about 40, about 20 to about 30, about 20 to about 25, at least about 8, at least about 10, at least about 15, at least about 20, at least about 25, at least about 30, at least about 32, at least about 35, at least about 40, at least about 44, at least about 50, at least about 60, at least about 70, at least about 80, at least about 90, at least about 100, at least about 110, at least about 120, at least about 130, at least about 140, at least about 150 nucleotides in length, or more, and any value or range therein.

In some embodiments, the identity of two or more spacer nucleotide sequences of a CRISPR array disclosed herein is the same. In some embodiments, the identity of two or more spacer nucleotide sequences of a CRISPR array disclosed herein is different. In some embodiments, the identities of two or more spacer nucleotide sequences of a CRISPR array are different but are complementary to one or more target nucleotide sequences. In some embodiments, the identities of two or more spacer nucleotide sequences of a CRISPR array are different and are complementary to one or more target nucleotide sequences that are overlapping sequences. In some embodiments, the identities of two or more spacer nucleotide sequences of a CRISPR array are different and are complementary to one or more target nucleotide sequences that are not overlapping sequences.

Codon Optimization

In some embodiments, a polynucleotide, nucleotide sequence and/or recombinant nucleic acid molecule described herein (e.g., polynucleotides comprising a CRISPR array, Cpf1 polypeptides, polynucleotides encoding transcriptional activators, and the like) is codon optimized for expression in any species of interest. Codon optimization involves modification of a nucleotide sequence for codon usage bias using species-specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species specific codon usage table with the codons present in the native polynucleotide sequences. Codon optimization of a nucleotide sequence results in a nucleotide sequence having less than 100% identity (e.g., 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to the native nucleotide sequence but which still encodes a polypeptide having the same function as that encoded by the original nucleotide sequence. In some embodiments, the nucleotide sequence and/or recombinant nucleic acid molecule of this disclosure are codon optimized for expression in the organism/species of interest.

Repeat Nucleotide Sequences

In some embodiments, a repeat nucleotide sequence of a CRISPR array comprises a nucleotide sequence of any known repeat nucleotide sequence of a CRISPR-Cpf1 system. In some embodiment, a repeat nucleotide sequence is of a synthetic sequence comprising the secondary structure of a native repeat from a CRISPR-Cpf1 system (e.g., an internal hairpin).

In some embodiments, a spacer nucleotide sequence of a CRISPR array described herein is linked at its 5′ end to the 3′ end of a repeat sequence. In some embodiments, the spacer nucleotide sequence is linked at its 5′ end to about 1 to about 8, about 1 to about 10, or about 1 to about 15 nucleotides of the 3′ end of a repeat nucleotide sequence. In some embodiments, the about 1 to about 8, about 1 to about 10, about 1 to about 15 nucleotides of the repeat nucleotide sequence are a portion of the 3′ end of a repeat nucleotide sequence. In some embodiments, spacer nucleotide sequence is linked at its 3′ end to the 5′ end of a repeat nucleotide sequence. In some embodiments, the spacer is linked at its 3′ end to about 1 to about 8, about 1 to about 10, or about 1 to about 15 nucleotides of the 5′ end of a repeat nucleotide sequence. In some embodiments, the about 1 to about 8, about 1 to about 10, about 1 to about 15 nucleotides of the repeat nucleotide sequence are a portion of the 5′ end of a repeat nucleotide sequence.

In some embodiments, a spacer nucleotide sequence described herein is linked at its 5′ end to a first repeat nucleotide sequence and linked at its 3′ end to a second repeat nucleotide sequence to form a repeat-spacer-repeat sequence. In some embodiments, a spacer described herein is linked at its 5′ end to about 1 to about 8, about 1 to about 10, or about 1 to about 15 nucleotides of the 3′ end of a first repeat sequence and is linked at its 3′ end to about 1 to about 8, about 1 to about 10, or about 1 to about 15 nucleotides of the 5′ end of a second repeat sequence. In some embodiments, the about 1 to about 8, about 1 to about 10, about 1 to about 15 nucleotides of the first repeat sequence are a portion of the 3′ end of the first repeat nucleotide sequence. In some embodiments, the about 1 to about 8, about 1 to about 10, about 1 to about 15 nucleotides of the first second sequence are a portion of the 3′ end of the second repeat nucleotide sequence. In some embodiments, a spacer nucleotide sequence disclosed herein is linked at its 5′ end to the 3′ end of a first repeat nucleotide sequence and is linked at its 3′ end to the 5′ of a second repeat nucleotide sequence where the spacer nucleotide sequence and the second repeat nucleotide sequence are repeated to form a repeat-(spacer-repeat)n sequence such that n is any integer from 1 to 100. Thus, in some embodiments, a repeat-(spacer-repeat)n sequence disclosed herein comprise, consist essentially of, or consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer nucleotide sequences.

Thus, in some embodiments, a repeat sequence is identical to or substantially identical to a repeat sequence from a wild-type Cpf1 loci. In some embodiments, a repeat sequence comprises a portion of a wild type repeat sequence (e.g., 1, 2, 3, 4, 5, 6, 7, 8, 9, 10, 11, 12, 13, 14, 15 or more contiguous nucleotides of a wild type repeat sequence). In some embodiments, a repeat sequence comprises, consists essentially of, or consists of at least one nucleotide (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, or more nucleotides, or any range therein).

Regulatory Elements

In some embodiments, recombinant CRISPR arrays, nucleotide sequences, and/or nucleic acid molecules disclosed herein are operatively associated with a variety of promoters, terminators and other regulatory elements for expression in various organisms or cells. In some embodiments, at least one promoter and/or terminator is operably linked to a recombinant nucleic acid molecule and/or a recombinant CRISPR array disclosed herein. Any promoter useful with this disclosure is used and includes, for example, promoters functional with the organism of interest as well as constitutive, inducible, developmental regulated, tissue-specific/preferred-promoters, and the like, as described herein. A regulatory element as used herein is endogenous or heterologous. In some embodiments, an endogenous regulatory element derived from the subject organism is inserted into a genetic context in which it does not naturally occur (e.g. a different position in the genome than as found in nature), thereby producing a recombinant or non-native nucleic acid.

In some embodiments, expression of a construct disclosed herein is constitutive, inducible, temporally regulated, developmentally regulated, or chemically regulated. In some embodiments, a construct is made constitutive, inducible, temporally regulated, developmentally regulated, or chemically regulated by operatively linking the construct to a promoter functional in an organism of interest. In some embodiments, repression is made reversible by operatively linking a recombinant nucleic acid construct disclosed herein to an inducible promoter that is functional in an organism of interest. The choice of promoter described herein will vary depending on the quantitative, temporal and spatial requirements for expression, and also depending on the host cell to be transformed.

Exemplary promoters for use with the methods, bacteriophage and composition disclosed herein include promoters that are functional in bacteria. For example, L-arabinose inducible (araBAD, P_{BAD}) promoter, any lac promoter, L-rhamnose inducible (rhaPBAD) promoter, T7 RNA polymerase promoter, trc promoter, tac promoter, lambda phage promoter (p_{L}p_{L}-9G-50), anhydrotetracycline-inducible (tetA) promoter, trp, Ipp, phoA, recA, proU, cst-1, cadA, nar, Ipp-lac, cspA, 11-lac operator, T3-lac operator, T4 gene 32, T5-lac operator, nprM-lac operator, Vhb, Protein A, corynebacterial-E. coli like promoters, thr, horn, diphtheria toxin promoter, sig A, sig B, nusG, SoxS, katb, a-amylase (Pamy), Ptms, P43 (comprised of two overlapping RNA polymerase a factor recognition sites, GA, GB), Ptms, P43, rplK-rplA, ferredoxin promoter, and/or xylose promoter.

In some embodiments, inducible promoters are used. In some embodiment, chemical-regulated promoters are used to modulate the expression of a gene in an organism through the application of an exogenous chemical regulator. The use of chemically regulated promoters enables RNAs and/or the polypeptides disclosed herein to be synthesized only when, for example, an organism is treated with the inducing chemicals. In some embodiments where a chemical-inducible promoter is used, the application of a chemical induces gene expression. In some embodiments wherein a chemical-repressible promoter is used, the application of the chemical represses gene expression. In some embodiments, the promoter is a light-inducible promoter, where application of specific wavelengths of light induces gene expression. In some embodiments, a promoter is a light-repressible promoter, where application of specific wavelengths of light represses gene expression.

Transformation

In some embodiments, the nucleotide sequences, constructs, and expression cassettes disclosed herein are expressed transiently and/or stably incorporated into the genome of a host organism. In some embodiments, a polynucleotide disclosed herein is introduced into a cell by any method known to those of skill in the art. Exemplary methods of transformation include transformation via electroporation of competent cells, passive uptake by competent cells, chemical transformation of competent cells, as well as any other electrical, chemical, physical (mechanical) and/or biological mechanism that results in the introduction of nucleic acid into a cell, including any combination thereof. In some embodiments, transformation of a cell comprises nuclear transformation. In some embodiments, transformation of a cell comprises plasmid transformation and conjugation.

In some embodiments, when more than one nucleotide sequence is introduced, the nucleotide sequences are assembled as part of a single nucleic acid construct, or as separate nucleic acid constructs, and are located on the same or different nucleic acid constructs. In some embodiments, nucleotide sequences are introduced into the cell of interest in a single transformation event, or in separate transformation events.

Expression Cassette

In some embodiments, a nucleic acid construct is an “expression cassette” or is in an expression cassette. As used herein, “expression cassette” means a recombinant nucleic acid molecule comprising a nucleotide sequence of interest (e.g., the recombinant nucleic acid molecules and CRISPR arrays disclosed herein), wherein the nucleotide sequence is operably associated with at least a control sequence (e.g., a promoter). In some embodiments, the expression cassettes are designed to express the recombinant nucleic acid molecules and/or the recombinant CRISPR arrays disclosed herein.

In some embodiments, an expression cassette comprising a nucleotide sequence of interest is chimeric, meaning that at least one of its components is heterologous with respect to at least one of its other components. In some embodiments, an expression cassette is naturally occurring but has been obtained in a recombinant form useful for heterologous expression.

In some embodiments, an expression cassette includes a transcriptional and/or translational termination region (i.e. termination region) that is functional in the selected host cell. In some embodiments, termination regions are responsible for the termination of transcription beyond the heterologous nucleotide sequence of interest and for correct mRNA polyadenylation. In some embodiments, the termination region is native to the transcriptional initiation region, is native to the operably linked nucleotide sequence of interest, is native to the host cell, or is derived from another source (i.e., foreign or heterologous to the promoter, to the nucleotide sequence of interest, to the host, or any combination thereof). In some embodiments, terminators are operably linked to the recombinant nucleic acid molecule and CRISPR array disclosed herein.

In some embodiments, an expression cassette includes a nucleotide sequence for a selectable marker. As used herein, “selectable marker” means a nucleotide sequence that when expressed imparts a distinct phenotype to the host cell expressing the marker and thus allows such transformed cells to be distinguished from those that do not have the marker. In some embodiments, a nucleotide sequence encode either a selectable or screenable marker, depending on whether the marker confers a trait that is selected for by chemical means, such as by using a selective agent (e.g. an antibiotic), or on whether the marker is simply a trait that one can identify through observation or testing, such as by screening (e.g., fluorescence).

Vectors

In addition to expression cassettes, the nucleic acid molecules and nucleotide sequences described herein (e.g. polynucleotides comprising a CRISPR array, polynucleotides encoding a transcriptional activator, polynucleotides encoding a Cpf1, or anti-CRISPR polypeptides) are used in connection with vectors. The term “vector” refers to a composition for transferring, delivering or introducing a nucleic acid (or nucleic acids) into a cell. A vector comprises a nucleic acid molecule comprising the nucleotide sequence(s) to be transferred, delivered or introduced. Non-limiting examples of general classes of vectors include but are not limited to a viral vector, a plasmid vector, a phage vector, a phagemid vector, a cosmid vector, a fosmid vector, a bacteriophage, an artificial chromosome, or an agrobacterium binary vector in double or single stranded linear or circular form which may or may not be self-transmissible or mobilizable. In some embodiments, the vector is a bacteriophage. In some embodiments, the vector is a plasmid.

A vector as defined herein transforms prokaryotic or eukaryotic host either by integration into the cellular genome or exist extrachromosomally (e.g. autonomous replicating plasmid with an origin of replication). Additionally included are shuttle vectors by which is meant a DNA vehicle capable, naturally or by design, of replication in two different host organisms. In some embodiments, a shuttle vector replicates in actinomycetes and bacteria and/or eukaryotes. In some embodiments, the nucleic acid in the vector are under the control of, and operably linked to, an appropriate promoter or other regulatory elements for transcription in a host cell. In some embodiments, the vector is a bi-functional expression vector which functions in multiple hosts.

In some embodiments, the vector comprises a first nucleic acid encoding a spacer sequence or a crRNA transcribed therefrom, wherein the spacer sequence is complimentary to a target nucleotide sequence from a target gene in the target bacterium. In some embodiments, the vector further comprises a second nucleic acid. In some embodiments, the second nucleic acid encodes a gene which inhibits DNA repair. In some embodiments, the second nucleic acid encodes an exogenous Cpf1. In some embodiments, the second nucleic acid encodes a transcriptional activator of the CRISPR-Cpf1 system. In other embodiments, the vector comprises both a second nucleic acid encoding a gene which inhibits DNA repair and a third nucleic acid encoding a transcriptional activator of the CRISPR-Cpf1 system. In some embodiments, the vector comprises a first nucleic acid encoding an exogenous Cpf1. In some embodiments, the vector comprises a first nucleic acid encoding transcriptional activator for the CRISPR-Cpf1 system in the target bacterium. In some embodiments, the vector comprises a first nucleic acid encoding an anti-CRISPR polypeptide.

In some embodiments, the gene which inhibits DNA repair is Gam. Gam is bacteriophage protein from Mu phage. In some embodiments, Gam binds to a DNA double stranded break where recA is bound and inhibit functionality of recA to enhance killing efficiency. In some embodiments, the vector further comprises a sequence encoding a Gam protein, also referred to herein as Gam or Mu-Gam. In some embodiments, expression of the Gam protein is controlled through a constitutive promoter. In some embodiments, the constitutive promoter controlling expression of the Gam protein further controls expression of the Cpf1 or the CRISPR array.

Described herein, in certain embodiments, are methods for killing a target bacterium comprising administering a vector comprising a first nucleic acid encoding a Gam protein and a second first nucleic acid encoding a spacer sequence or a crRNA transcribed therefrom, wherein the spacer sequence is complimentary to a target nucleotide sequence from a target gene in the target bacterium. In some embodiments, the spacer sequence is a multiplex spacer sequence.

### Repair Mechanism

In some embodiments, a CRISPR-Cas system disclosed herein causes a nucleic acid double strand break (DNA double strand break/cleavage). In some embodiments, double strand breaks are repaired, for example by non-homologous end joining, microhomology-mediated end joining, or homology-directed repair. Non-homologous end joining refers to the repair of a double strand breaks in DNA by direct ligation of one end of the break to the other end of the break without a requirement for a donor polynucleotide. In some embodiments, DNA ligase IV forms a complex with cofactor XRCC4 to directly join two ends of a DNA break. Homology-directed repair relies on the presence of a template for repair. In some examples of genome engineering, a donor polynucleotide or portion thereof is inserted into the break. In some embodiments, a RecA initiates the repair of double-stranded DNA breaks. In some embodiments, AddAB initiates the repair of double-stranded DNA breaks. In some embodiments, a RecBCD enzyme initiates the repair of double-stranded DNA breaks by homologous recombination. In some embodiments, an enzyme that repair double strand breaks is a helicase-nuclease. In some embodiments, Ligase A is involved in double strand break repair.

In some embodiments, a system described herein is used to deliver an inhibitor of double strand break repair. In some embodiments, a system described herein is used to deliver a CRISPR-Cpf1 system and an inhibitor of double strand break repair. In some embodiments, an exogenous molecule inhibits DNA repair. In some embodiments, the molecule is an exogenous protein that binds the ends of the double stranded break and inhibits double strand break repair. In some embodiments, the protein is a Mu phage Gam protein. In some embodiments, the protein is a lambda phage Gam protein. In some embodiments, the protein is a phage T7 gp5.9 protein. In some embodiments, the protein is a RecA, recBCD or AddAB inhibitor. In some embodiments, the protein inhibits RecA activity. In some embodiments, the protein inhibits recBCD activity. In some embodiments, the protein inhibits AddAB activity. In some embodiments, a protein described herein that inhibit double strand break repair is expressed by the target bacteria or a bacteriophage disclosed herein. In some embodiments, described herein are methods for killing a target bacterium comprising: introducing into a target bacterium a bacteriophage comprising: a first nucleic acid encoding a spacer sequence or a crRNA transcribed therefrom, wherein the spacer sequence is complimentary to a target nucleotide sequence from a target gene in the target bacterium; and a second nucleic acid encoding a protein that inhibits double strand break repair. In some embodiments, a bacteriophage disclosed herein comprises one or more compositions, for example a small organic molecule, peptide or nucleic acid, which inhibits, reduces or abolishes the double strand break repair.

### Bacteriophages

Bacteriophages or “phages” represent a group of bacterial viruses and are engineered or sourced from environmental sources. Individual bacteriophage host ranges are usually narrow, meaning, phages are highly specific to one strain or few strains of a bacterial species and this specificity makes them unique in their antibacterial action. Bacteriophages are bacterial viruses that rely on the host's cellular machinery to replicate. Generally, phages generally fall into three categories: lytic, lysogenic, and temperate. Lytic bacteriophages infect a host cell, undergo numerous rounds of replication, and trigger cell lysis to release newly made bacteriophage particles. Lysogenic bacteriophages permanently reside within the host cell, either within the bacterial genome or as an extrachromosomal plasmid. Temperate bacteriophages are capable of being lytic or lysogenic, and choose one versus the other depending on growth conditions and the physiological state of the cell. Anytime a lysogenic bacterium is exposed to adverse conditions, in some embodiments, the lysogenic state is terminated. This process is called induction. Adverse conditions which favor the termination of the lysogenic state include desiccation, exposure to UV or ionizing radiation, and exposure to mutagenic chemicals. This leads to the expression of the phage genes, reversal of the integration process, and lytic multiplication.

Bacteriophages package and deliver synthetic DNA using three general approaches. Under the first approach, the synthetic DNA is randomly recombined into the bacteriophage genome, which usually involves a selectable marker. Under the second approach, restriction sites within the phage are used to introduce synthetic DNA in-vitro. Under the third approach, a plasmid generally encoding the phage packaging sites and lytic origin of replication is packaged as part of the assembly of the bacteriophage particle. The resulting plasmids have been coined “phagemids.”

Phages are limited to a given bacterial strain for evolutionary reasons. Injecting their genetic material into an incompatible strain is counterproductive. Phages have therefore evolved to specifically infect a limited cross-section of strains. However, some phages have been discovered that inject their genetic material into a wide range of bacteria. The classic example is the PI phage, which has been shown to inject DNA in a range of gram-negative bacteria.

In some embodiments, the bacteriophage or phagemid DNA is from a lysogenic or temperate bacteriophage. In some embodiments, the bacteriophage or phagemid DNA is from an obligate lytic bacteriophage. In some embodiments, the bacteriophages or phagemids include but are not limited to PI phage, a Ml 3 phage, a λ phage, a T4 phage, a ϕC2 phage, a ϕCD27 phage, a ϕNMl phage, Bc431 v3 phage, ϕ10 phage, ϕ25 phage, ϕ151 phage, A511-like phages, B054, 0176-like phages, or Campylobacter phages (such as NCTC 12676 and NCTC 12677). In some embodiments, the bacteriophage is φCD146 C. difficile bacteriophage. In some embodiments, the bacteriophage is φCD24-2 C. difficile bacteriophage. In some embodiments, the bacteriophage is T4 E. coli bacteriophage. In some embodiments, the bacteriophage is T7 E. coli bacteriophage. In some embodiments, the bacteriophage is T7m E. coli bacteriophage.

In some embodiments, a plurality of bacteriophages are used together. In some embodiments, the plurality of bacteriophages used together targets the same or different bacteria within a sample or subject. In some embodiments, the bacteriophages used together comprises T4 phage, T7 phage, T7m phage, or any combination of bacteriophages described herein.

In some embodiments, bacteriophages of interest are obtained from environmental sources. or commercial research vendors. In some embodiments, obtained bacteriophages are screened for lytic activity against a library of bacteria and their associated strains. In some embodiments, the bacteriophages are screened against a library of bacteria and their associated strains for their ability to generate primary resistance in the screened bacteria.

In some embodiments, disclosed herein are method for killing a target bacterium comprising introducing into a target bacterium a bacteriophage comprising: a nucleic acid encoding a spacer sequence or a crRNA transcribed therefrom, wherein the spacer sequence is complimentary to a target nucleotide sequence from a target gene in the target bacterium; and a gene that is capable of inducing lysis of the target bacterium, wherein the target bacterium is killed by lytic activity of the bacteriophage or activity of a CRISPR-Cpf1 system using the spacer sequence or the crRNA transcribed therefrom. In some embodiments, disclosed herein are bacteriophages comprising: a nucleic acid encoding a spacer sequence or a crRNA transcribed therefrom, wherein the spacer sequence is complimentary to a target nucleotide sequence from a target gene in a target bacterium; and a gene that is capable of inducing lysis of the target bacterium, wherein the target bacterium is killed by the lytic activity of the bacteriophage or activity of a CRISPR-Cpf1 system using the spacer sequence or the crRNA transcribed therefrom.

Insertion Sites

In some embodiments, the introduction of a nucleic acid encoding a CRISPR array into a bacteriophage does not disrupt the lytic activity of the bacteriophage. In some embodiments, the introduction of a nucleic acid encoding a CRISPR array into a bacteriophage preserves the lytic activity of the bacteriophage. In some embodiments, the nucleic acid is inserted into the bacteriophage genome. In some embodiments, the nucleic acid is inserted into the bacteriophage genome at a transcription terminator site at the end of an operon of interest. In some embodiments, the nucleic acid is inserted into the bacteriophage genome as a replacement for one or more removed non-essential genes. In some embodiments, the nucleic acid is inserted into the bacteriophage genome as a replacement for one or more removed lysogenic genes. In some embodiments, the replacement of non-essential and/or lysogenic genes with the nucleic acid does not affect the lytic activity of the bacteriophage. In some embodiments, the replacement of non-essential and/or lysogenic genes with the nucleic acid preserves the lytic activity of the bacteriophage. In some embodiments, the replacement of non-essential and/or lysogenic genes with the nucleic acid enhances the lytic activity of the bacteriophage. In some embodiments, the replacement of non-essential and/or lysogenic genes with the nucleic acid renders a lysogenic bacteriophage lytic.

In some embodiments, the nucleic acid is introduced into the bacteriophage genome at a first location while one or more non-essential and/or lysogenic genes are separately removed and/or inactivated from the bacteriophage genome at a separate location. In some embodiments, the removal and/or inactivation of one or more non-essential and/or lysogenic genes does not affect the lytic activity of the bacteriophage. In some embodiments, the removal and/or inactivation of one or more non-essential and/or lysogenic genes preserves the lytic activity of the bacteriophage. In some embodiments, the removal of one or more non-essential and/or lysogenic genes renders a lysogenic bacteriophage into a lytic bacteriophage. Similarly, in some embodiments, one or more lytic genes are introduced into the bacteriophage so as to render a non-lytic, lysogenic bacteriophage into a lytic bacteriophage.

In some embodiments, the bacteriophage is a temperate bacteriophage which has been rendered lytic by any of the aforementioned means. In some embodiments, a temperate bacteriophage is rendered lytic by the removal, replacement, or inactivation of one or more lysogenic genes. In some embodiments, the lytic activity of the bacteriophage is due to the removal, replacement, or inactivation of at least one lysogeny gene. In some embodiments, a temperate bacteriophage is rendered lytic by the removal, replacement, or inactivation of one or more lysogenic gene and comprises a CRISPR array comprising at least one spacer that is complementary to a target nucleotide sequence in a target gene in a target bacterium. In some embodiments, a temperate bacteriophage is rendered lytic by the removal, replacement, or inactivation of one or more lysogenic gene via a CRISPR array comprising a spacer directed to the one or more lysogenic gene and comprises a CRISPR array comprising at least one spacer that is complementary to a target nucleotide sequence in a target gene in a target bacterium. In some embodiments, the lysogenic gene plays a role in the maintenance of lysogenic cycle in the bacteriophage. In some embodiments, the lysogenic gene plays a role in establishing the lysogenic cycle in the bacteriophage. In some embodiments, the lysogenic gene plays a role in both establishing the lysogenic cycle and in the maintenance of the lysogenic cycle in the bacteriophage. In some embodiments, the lysogenic gene is a repressor gene. In some embodiments, the lysogenic gene is cI repressor gene. In some embodiments, the lysogenic gene is an activator gene. In some embodiments, the lysogenic gene is cII gene. In some embodiments, the lysogenic gene is lexA gene. In some embodiments, the lysogenic gene is int (integrase) gene. In some embodiments, two or more lysogeny genes are removed, replaced, or inactivated to cause arrest of a bacteriophage lysogeny cycle and/or induction of a lytic cycle. In some embodiments, a temperate bacteriophage is rendered lytic by the insertion of one or more lytic genes. In some embodiments, a temperate bacteriophage is rendered lytic by the insertion of one or more genes that contribute to the induction of a lytic cycle. In some embodiments, a temperate bacteriophage is rendered lytic by altering the expression of one or more genes that contribute to the induction of a lytic cycle. In some embodiments, a temperate bacteriophage phenotypically changes from a lysogenic bacteriophage to a lytic bacteriophage. In some embodiments, a temperate bacteriophage is rendered lytic by environmental alterations. In some embodiments, environmental alterations include, but are not limited to, alterations in temperature, pH, or nutrients, exposure to antibiotics, hydrogen peroxide, foreign DNA, or DNA damaging agents, presence of organic carbon, and presence of heavy metal (e.g. in the form of chromium (VI). In some embodiments, a temperate bacteriophage that is rendered lytic is prevented from reverting to lysogenic state. In some embodiments, a temperate bacteriophage that is rendered lytic is prevented from reverting back to lysogenic state by way of introducing an additions CRIPSR array. In some embodiments, the bacteriophage does not confer any new properties onto the target bacterium beyond cellular death cause by lytic activity of the bacteriophage and/or the activity of the CRISPR array.

In some embodiments, the replacement, removal, inactivation, or any combination thereof, of one or more non-essential and/or lysogenic genes is achieved by chemical, biochemical, and/or any suitable method. In some embodiments, the insertion of one or more lytic genes is achieved by any suitable chemical, biochemical, and/or physical method by homologous recombination.

In some embodiments, the bacteriophage is an obligate lytic bacteriophage. In some embodiments, the bacteriophage is ϕCD146 C. difficile bacteriophage. In some embodiments, the bacteriophage is ϕCD24-2 C. difficile bacteriophage. In some embodiments, the bacteriophage is T4 E. coli bacteriophage. In some embodiments, the bacteriophage is T7 E. coli bacteriophage. In some embodiments, the bacteriophage is T7m E. coli bacteriophage.

Non Essential Gene

In some embodiments, the non-essential gene to be removed and/or replaced from the bacteriophage is gp49 from ϕCD146 C. difficile bacteriophage. In some embodiments, the non-essential gene to be removed and/or replaced from the bacteriophage is gp75 from ϕCD24-2C. difficile bacteriophage. In some embodiments, the non-essential gene to be removed and/or replaced from the bacteriophage is the hoc gene from a T4 E. coli bacteriophage. In some embodiments, the non-essential gene to be removed and/or replaced include gp0.7, gp4.3, gp4.5, gp4.7, or any combination thereof from a T7 E. coli bacteriophage. In some embodiments, the non-essential gene to be removed and/or replaced is gp0.6, gp0.65, gp0.7, gp4.3, gp4.5, or any combination thereof from a T7m E. coli bacteriophage.

Enhanced Lytic Bacteriophages

Disclosed herein, are methods of producing a bacteriophage that comprises a nucleic acid encoding an exogenous Cpf1. Also, disclosed herein, are bacteriophages that comprises a nucleic acid encoding an exogenous Cpf1.

Disclosed herein, are methods of producing a bacteriophage that comprises a nucleic acid encoding a transcriptional activator for a CRISPR-Cpf1 system. Also, disclosed herein, are bacteriophages that comprises a nucleic acid encoding a transcriptional activator for a CRISPR-Cpf1 system.

In some embodiments, the introduction of a nucleic acid encoding a transcriptional activator for a CRISPR-Cpf1 into a bacteriophage is used to modulate the activity of a CRISPR-Cpf1 system in the target bacterium. In some embodiments, the transcriptional activator introduced by the bacteriophage increases the expression of a CRISPR-Cpf1 system in the target bacterium. In some embodiments, the increased expression of a CRISPR-Cpf1 system in the target bacterium due to the introduction of a transcriptional activator by a first bacteriophage, enhances the lethality of a second different bacteriophage comprising a CRISPR array as described by previous embodiments. In some embodiments, the increased expression of a CRISPR-Cpf1 system in the target bacterium due to the introduction of a transcriptional activator by a first bacteriophage, enhances the lethality of a second different bacteriophage comprising a pre-processed immature or a processed mature crRNA as described by previous embodiments.

In addition to regulation by transcriptional activators, the CRISPR-Cpf1 system is tightly controlled by other means and mechanisms of regulation. Quorum sensing (QS) is the chemical communication between bacteria within a bacterial population which permits the coordination of gene expression with respect to the population density. QS relies upon chemical signals that are produced and accumulate during bacterial growth. Upon hitting a threshold level, QS signals bind to transcriptional regulators to influence bacterial gene expression. In some bacteria, QS signaling enhances the CRISPR-Cpf1 system for bacterial defense by de-repressing its expression. In addition to QS signaling, the regulation of CRISPR-Cpf1 system expression is believed to be sensitive to perturbations in the host bacterium's membrane integrity.

In some embodiments, the transcriptional activator comprises a QS signal. In some embodiments, the transcriptional activator comprises a protein involved in sensing stress to the membrane of the host bacterium. In some embodiments, the transcriptional activator comprises a protein which stabilizes Cpf1. In some embodiments, the transcriptional activator is a metabolic sensing protein. In some embodiments, a nucleic acid encoding a transcriptional activator or a functional fragment thereof is introduced into the target bacteria. In some embodiments, a nucleic acid encoding a transcriptional activator or a functional fragment thereof is introduced into the target bacteria via a CRISPR array described herein. In some embodiments, the methods disclosed herein comprises: introducing a bacteriophage comprising a nucleic acid encoding a transcriptional activator for the CRISPR-Cpf1 system in the target bacterium. In some embodiments, disclosed herein are bacteriophages comprising a nucleic acid encoding a transcriptional activator for a CRISPR-Cpf1 system in a target bacterium.

### Anti-CRISPR Array

In some embodiments, a bacteriophage disclosed herein further comprises an Anti-CRISPR.

In some embodiments, a method disclosed herein comprises introducing into a target bacterium a bacteriophage comprising: lytic activity, and a first nucleic acid sequence encoding an anti-CRISPR polypeptide, wherein the anti-CRISPR polypeptide enhances the lytic activity of the bacteriophage. In some embodiments, disclosed herein are bacteriophages comprising: lytic activity, and a first nucleic acid sequence encoding an anti-CRISPR polypeptide, wherein the anti-CRISPR polypeptide enhances the lytic activity of the bacteriophage.

In some embodiments, the nucleic acid encoding an anti-CRISPR polypeptide directly enhances the lytic activity of the bacteriophage or another bacteriophage. In some embodiments, enhancement of the lytic activity of the bacteriophage is due to the anti-CRISPR polypeptide inhibiting, inactivating, and/or repressing the activity of a CRISPR-Cpf1 system in the host target bacterium. An anti-CRISPR polypeptide is any bacteriophage protein with activity that prevents the function of a bacterial CRISPR-Cpf1 system. Activity of an anti-CRISPR protein prevents a host bacterium from mounting a CRISPR-Cpf1 system based defense against the invading bacteriophage. In some embodiments, the anti-CRISPR polypeptide inactivates the host bacterium's CRISPR-Cpf1 system using a process comprising gene regulation interference. In some embodiments, the anti-CRISPR polypeptide inactivates the host bacterium's CRISPR-Cpf1 system using a process comprising nuclease recruitment interference. In some embodiments, the anti-CRISPR polypeptide inhibits, inactivates, and/or represses the activity of a CRISPR-Cpf1 system.

In some embodiments, the anti-CRISPR polypeptide is a truncated, mutated, or fused to another protein of interest. In some embodiments, the anti-CRISPR polypeptide is a dimer protein. In some embodiments, the anti-CRISPR polypeptide is a homodimer or heterodimer protein. In one embodiment, the anti-CRISPR polypeptide comprises AcrIIC1Boe, AcrIIC1Nme, AcrIIC2Nme, AcrIIC3Nme, AcrIIC4Hpa, AcrIIC5Smu, or any functional fragments thereof. In one embodiment, the anti-CRISPR polypeptide binds with specific affinity to a specific binding site upon the CRISPR-Cpf1 system.

In some embodiments, the anti-CRISPR polypeptide inhibits, inactivates, or represses the activity of a CRISPR-Cpf1 system in the target bacterium, wherein said CRISPR-Cpf1 system targets the bacteriophage comprising the nucleic acid encoding the anti-CRISPR polypeptide. In some embodiments, the anti-CRISPR polypeptide inhibits, inactivates, or represses the activity of a CRISPR-Cpf1 system in the target bacterium, wherein said CRISPR-Cpf1 system targets a second orthogonal bacteriophage different than a first bacteriophage. In some embodiments, the second orthogonal bacteriophage is different than the first bacteriophage. In some embodiments, the inhibition, inactivation, or repression of the CRISPR-Cpf1 system activity in the target bacterium by the anti-CRISPR polypeptide from a first bacteriophage enhances the activity of the first bacteriophage or a second orthogonal bacteriophage. In some embodiments, the second orthogonal bacteriophage has lytic activity. In some embodiments, the second orthogonal bacteriophage comprises a bacteriophage of any of the embodiments disclosed herein.

### Methods of Killing a Target Bacterium

Disclosed herein, in certain embodiments, are methods of killing bacteria. In some embodiments, killing of the target bacterium is achieved by the lytic activity of the bacteriophage. In some embodiments, killing of a target bacterium is achieved by the activity of a CRISPR array comprising at least one spacer that is complimentary to a target nucleotide sequence in a target gene in the target bacterium. In some embodiments, killing of the target bacterium is achieved by the activity of a mature crRNA. In some embodiments, killing of the bacterium is achieved by the processing of the CRISPR array by a CRISPR-Cpf1 system to produce a processed crRNA capable of directing CRISPR-Cpf1 based endonuclease activity and/or cleavage at the target nucleotide sequence in the target gene of the bacterium. In some embodiments, killing of a target bacterium is achieved by the activity of the CRISPR array independent to the lytic and/or non-lytic activity of the bacteriophage. In some embodiments, the killing of a target bacterium is by any method or combination of methods disclosed herein.

In some embodiments, killing of the bacterium are achieved solely by the lytic activity of the bacteriophage. In some embodiments, killing of the bacterium is achieved solely by the activity of the nucleic acid encoding a CRISPR array comprising at least one spacer. In some embodiments, killing of the bacterium is achieved solely by the activity of the nucleic acid encoding a mature crRNA. In some embodiments, killing of the bacterium is achieved by a combination of the lytic activity of the bacteriophage and the activity of the CRISPR array or mature crRNA. In some embodiments, killing of the bacterium by a combination of the lytic activity of the bacteriophage and by the activity of the first nucleic acid encoding a CRISPR array is synergistic. In some embodiments, the killing activity of the CRISPR array or mature crRNA supplements or enhances the lytic activity of the bacteriophage. In some embodiments, killing of a target bacterium is a synergistic effect of two or more systems.

In some embodiments, the synergistic killing of the bacterium is modulated by the concentration of the bacteriophage and/or the design of the CRISPR array. In some embodiments, the synergistic killing of the bacterium is modulated to favor killing by the lytic activity of the bacteriophage over the activity of the CRISPR array by increasing the concentration of bacteriophage administered to the bacterium. In some embodiments, the synergistic killing of the bacterium is modulated to disfavor killing by the lytic activity of the bacteriophage over the activity of the CRISPR array by decreasing the concentration of bacteriophage administered to the bacterium. In some embodiments, at low concentrations, lytic replication allows for amplification and killing of the target bacteria. In some embodiments, at high concentrations, amplification of a phage is not required.

In some embodiments, the synergistic killing of the bacterium is modulated to favor killing by the activity of the CRISPR array over the lytic activity of the bacteriophage by altering the number, the length, the composition, the identity, or any combination thereof, of the spacers so as to increase the lethality of the CRISPR array. In some embodiments, the synergistic killing of the bacterium is modulated to disfavor killing by the activity of the CRISPR array over the lytic activity of the bacteriophage by altering the number, the length, the composition, the identity, or any combination thereof, of the spacers so as to decrease the lethality of the CRISPR array.

In some embodiments, the target nucleotide sequence in the bacterium to be killed is any essential target nucleotide sequence of interest. In some embodiments, the target nucleotide sequence is a non-essential sequence. In some embodiments, a target nucleotide sequence comprises, consists essentially of or consist of all or a part of a nucleotide sequence encoding a promoter, or a complement thereof, of a target gene. In some embodiments, the spacer nucleotide sequence is complementary to a promoter, or a part thereof, of a target gene.

In some embodiments, the target nucleotide sequence comprises all or a part of a nucleotide sequence located on a coding or a non-coding strand of DNA. In some embodiments, the target nucleotide sequence comprises all or a part of a nucleotide sequence located on a coding of a transcribed region of a target gene.

An essential gene is any gene of an organism that is critical for its survival. However, being essential is highly dependent on the circumstances in which an organism lives. For instance, a gene required to digest starch is only essential if starch is the only source of energy. In some embodiments, the essential gene includes but is not limited to: yfaP, speA, ftsZ, acpP, csrA, eno, fusA, gapA, glyQ, infA, nusG, secY, trmD, Tsf, ftsA or homologues thereof. In some embodiments, a non-essential gene is any gene of an organism that is not critical for survival. Examples of non-essential genes include, but are not limited to, treF, eamB, irhA, lacZ, soxS, rdgC, zwfl, acnA or homologues thereof. However, being non-essential is highly dependent on the circumstances in which an organism lives.

In some embodiments, non-limiting examples of a target gene of interest includes a gene encoding a transcriptional regulator, a translational regulator, a polymerase gene, a metabolic enzyme, a transporter, an RNase, a protease, a DNA replication enzyme, a DNA modifying or degrading enzyme, a regulatory RNA, a transfer RNA, or a ribosomal RNA. In some embodiments, a target gene is a gene involved in cell-division, cell structure, metabolism, motility, pathogenicity or virulence. In some embodiments, a target gene includes a hypothetical gene whose function is not yet characterized. Thus, for example, the target genes are any gene from any bacterium.

Antimicrobial Agents and Peptides

In some embodiments, a bacteriophage disclosed herein is further genetically modified to express an antibacterial peptide, a functional fragment of an antibacterial peptide or a lytic gene. In some embodiments, a bacteriophage disclosed herein express at least one antimicrobial agent or peptide disclosed herein. In some embodiments, a bacteriophage disclosed herein comprises a nucleic acid sequence that encodes an enzybiotic where the protein product of the nucleic acid sequence targets phage resistant bacteria. In some embodiments, the bacteriophage comprises nucleic acids which encode enzymes which assist in breaking down or degrading biofilm matrix. In some embodiments, a bacteriophage disclosed herein comprises nucleic acids encoding Dispersin D aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, xylanase or lyase. In some embodiments, the enzyme is selected from the group consisting of cellulases, such as glycosyl hydroxylase family of cellulases, such as glycosyl hydroxylase 5 family of enzymes also called cellulase A; polyglucosamine (PGA) depolymerases; and colonic acid depolymerases, such as 1,4-L-fucodise hydrolase Characterisation of a 1,4-beta-fucoside hydrolase degrading colanic acid, depolymerazing alginase, DNase I, or combinations thereof. In some embodiments, a bacteriophage disclosed herein secretes an enzyme disclosed herein.

In some embodiments, an antimicrobial agent or peptide is expressed and/or secreted by a bacteriophage disclosed herein. In some embodiments, a bacteriophage disclosed herein secretes and expresses an antibiotic such as ampicillin, penicillin, penicillin derivatives, cephalosporins, monobactams, carbapenems, ofloxacin, ciproflaxacin, levofloxacin, gatifloxacin, norfloxacin, lomefloxacin, trovafloxacin, moxifloxacin, sparfloxacin, gemifloxacin, pazufloxacin or any antibiotic disclosed herein. In some embodiments, a bacteriophage disclosed herein comprises a nucleic acid sequence encoding an antibacterial peptide, expresses an antibacterial peptide, or secretes a peptide that aids or enhances killing of a target bacterium. In some embodiments, a bacteriophage disclosed herein comprises a nucleic acid sequence encoding a peptide, a nucleic acid sequence encoding an antibacterial peptide, expresses an antibacterial peptide, or secretes a peptide that aids or enhances the activity of a CRISPR-Cpf1 system. In some embodiments, a bacteriophage disclosed herein comprises a nucleic acid sequence encoding a peptide. In some embodiments, a bacteriophage disclosed herein comprises a nucleic acid sequence encoding an antibacterial peptide. In some embodiments, a bacteriophage disclosed herein expresses an antibacterial peptide. In some embodiments, a bacteriophage secretes a peptide that aids or enhances the activity of a CRISPR-Cpf1 system.

### Uses

Bacterial Infections

Disclosed herein, are methods of treating bacterial infections. In some embodiments, the bacteriophages disclosed herein treat or prevent diseases or conditions mediated or caused by bacteria as disclosed herein in a human or animal subjects. Such bacteria are typically in contact with tissue of the subject including: gut, oral cavity, lung, armpit, ocular, vaginal, anal, ear, nose or throat tissue. In some embodiments, a bacterial infection is treated by modulating the activity of the bacteria and/or by directly killing of the bacteria.

In some embodiments, one or more target bacteria present in a bacterial population are pathogenic. In some embodiments, the pathogenic bacteria are uropathogenic. In some embodiments, the pathogenic bacterium is uropathogenic E. Coli (UPEC). In some embodiments, the pathogenic bacteria are diarrheagenic. In some embodiments, the pathogenic bacteria are diarrheagenic E. coli (DEC). In some embodiments, the pathogenic bacteria are Shiga-toxin producing. In some embodiments, the pathogenic bacterium is Shiga-toxin producing E. coli (STEC). In some embodiments, the pathogenic bacteria are Shiga-toxin producing. In some embodiments, the pathogenic bacterium is Shiga-toxin producing E. coli (STEC). In some embodiments, the pathogenic bacterium is Shiga-toxin producing E. coli (STEC). In some embodiments, the pathogenic bacteria are various O-antigen:H-antigen serotype E. coli. In some embodiments, the pathogenic bacteria are enteropathogenic. In some embodiments, the pathogenic bacterium is enteropathogenic E. coli (EPEC).

In some embodiments, the pathogenic bacteria are various strains of C. difficile including: CD043, CD05, CD073, CD093, CD180, CD106, CD128, CD199, CD111, CD108, CD25, CD148, CD154, FOBT195, CD03, CD038, CD112, CD196, CD105, UK1, UK6, BI-9, CD041, CD042, CD046, CD19, or R20291.

In some embodiments, the bacteriophages disclosed herein are used to treat an infection, a disease, or a condition, in the gastrointestinal tract of a subject. In some embodiments, the bacteriophages are used to modulate and/or kill target bacteria within the microbiome or gut flora of a subject. In some embodiments, the bacteriophages are used to selectively modulate and/or kill one or more target bacteria from a plurality of bacteria within the microbiome or gut flora of a subject. In some embodiments, the bacteriophages are used to selectively modulate and/or kill one or more target enteropathogenic bacteria from a plurality of bacteria within the microbiome or gut flora of a subject. In some embodiments, the target enteropathogenic bacterium is enteropathogenic E. Coli (EPEC). In some embodiments, the bacteriophages are used to selectively modulate and/or kill one or more target diarrheagenic bacteria from a plurality of bacteria within the microbiome or gut flora of a subject. In some embodiments, the target diarrheagenic bacterium is diarrheagenic E. coli (DEC). In some embodiments, the bacteriophages are used to selectively modulate and/or kill one or more target Shiga-toxin producing bacteria from a plurality of bacteria within the microbiome or gut flora of a subject. In some embodiments, the target Shiga-toxin producing bacterium is Shiga-toxin producing E. coli (STEC).

In some embodiments, the bacteriophages are used to selectively modulate and/or kill one or more target enteropathogenic C. difficile bacteria strains within the microbiome or gut flora of a subject including: CD043, CD05, CD073, CD093, CD180, CD106, CD128, CD199, CD111, CD108, CD25, CD148, CD154, FOBT195, CD03, CD038, CD112, CD196, CD105, UK1, UK6, BI-9, CD041, CD042, CD046, CD19, or R20291.

In some embodiments, the bacteriophages disclosed herein are used to treat an infection, a disease, or a condition, in the urinary tract of a subject. In some embodiments, the bacteriophages are used to modulate and/or kill target bacteria within the urinary tract flora of a subject. The urinary tract flora includes, but is not limited, to Staphylococcus epidermidis, Enterococcus faecalis, and some alpha-hemolytic Streptococci. In some embodiments, the bacteriophages are used to selectively modulate and/or kill one or more target uropathogenic bacteria from a plurality of bacteria within the urinary tract flora of a subject. In some embodiments, the target bacterium is uropathogenic E. Coli (UPEC).

In some embodiments, the bacteriophages disclosed herein are used to treat an infection, a disease, or a condition, on the skin of a subject. In some embodiments, the bacteriophages are used to modulate and/or kill target bacteria on the skin of a subject.

In some embodiments, the bacteriophages disclosed herein are used to treat an infection, a disease, or a condition, on a mucosal membrane of a subject. In some embodiments, the bacteriophages are used to modulate and/or kill target bacteria on the mucosal membrane of a subject.

In some embodiments, the pathogenic bacteria are antibiotic resistant. In one embodiment, the pathogenic bacterium is methicillin-resistant Staphylococcus aureus (MRSA).

In some embodiments, the one or more target bacteria present in the bacterial population form a biofilm. In some embodiments, the biofilm comprises pathogenic bacteria. In some embodiments, the bacteriophage disclosed herein is used to treat a biofilm.

In some embodiments, the target bacteria is a gram negative bacteria. In some embodiments, a gram negative bacteria is a bacteria in the family Enterobacteriaceae. In some embodiments, the enterobacteriaceae is carbapenem-resistant Enterobacteriaceae. In some embodiments, the target bacteria is a cyanobacteria. In some embodiments, non-limiting examples of target bacteria include bacterial species selected from a genus comprising: Actinomyces, Acinetobacter, Bacillus, Burkholderia, Corynebacterium, Campylobacter, Clostridium, Clostridium, Escherichia, Enterococcus, Haemophilis, Helicobacter, Klebsiella., Lactococcus, Mycobacterium, Myxococcus, Neisseria, Porphyromonas, Prevotella, Pseudomonas, Salmonella, Serratia, Shigella, Staphylococcus, or Streptococcus. In some embodiments, the Corynebacterium is Corynebacterium group G1 or Corynebacterium group G2. In some embodiments, the bacteria is Escherichia coli, Salmonella enterica, Shigella dysenteriae, Bacillus subtilis, Clostridium acetobutylicum, Clostridium ljungdahlii, Clostridium difficile, Acinetobacter baumannii, Mycobacterium tuberculosis, Myxococcus xanthus, Staphylococcus aureus, Streptococcus pyogenes, Staphylococcus aureus, Streptococcus pneumonia, Staphylococcus epidermidis, Staphylococcus salivarius, Corynebacterium minutissium, Corynebacterium pseudodiphtherias, Corynebacterium stratium, Streptococcus pneumonia, Streptococcus mitis, Streptococcus sanguis, Klebsiella pneumoniae, Pseudomonas aeruginosa, Burkholderia cepacia, Serratia marcescens, Haemophilus influenzae, Neisseria meningitidis, Neisseria gonorrhoeae, Salmonella typhimurium, Prevotella melaninogenicus, Helicobacter pylori, Helicobacter felis, or Campylobacter jejuni. In some embodiments, the bacterium is a drug resistant bacteria. In some embodiments, the drug resistant bacterium is resistant to at least one antibiotic. In some embodiments, the antibiotic is a cephalosporin, a fluoroquinolone, a carbapenem, a colistin, an aminoglycoside, vancomycin, streptomycin, or methicillin. A non-limiting example of a drug resistant bacterium is a methicillin resistant Staphylococcus aureus. Further non-limiting examples of target bacteria include lactic acid bacteria including but not limited to Lactobacillus spp. and Bifidobacterium spp.; electrofuel bacterial strains including but not limited to Geobacter spp., Clostridium spp., or Ralstonia eutropha; or bacteria pathogenic on, for example, plants and mammals.

In some embodiments, the target bacterium is Escherichia coli. In some embodiments, the target bacterium is Clostridium difficile. In some embodiments, the target bacterium is Klebsiella pneumoniae. In some embodiments, the target bacterium is Salmonella enterica. In some embodiments, the target bacterium is Shigella dysenteriae. In some embodiments, the target bacterium is Staphylococcus aureus. In some embodiments, the target bacterium is Clostridium bolteae. In some embodiments, the target bacterium is the genus Enterococcus. In some embodiments, the target bacterium is in the genus Acinetobacter. In some embodiments, the target bacterium is in the genus Pseudomonas. In some embodiments, the methods and compositions disclosed herein are for use in veterinary and medical applications as well as research applications

In some embodiments, the bacteriophage treats acne and other related skin infections.

In some embodiments, a target bacterium is a multiple drug resistant (MDR) bacteria strain. An MDR strain is a bacteria strain that is resistant to at least one antibiotic. In some embodiments, a bacteria strain is resistant to an antibiotic class such as a cephalosporin, a fluoroquinolone, a carbapenem, a colistin, an aminoglycoside, vancomycin, streptomycin, and methicillin. In some embodiments, a bacteria strain is resistant to an antibiotic such as a Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Linezolid, a Streptogramin, Tigecycline, Daptomycin, or any combination thereof. Examples of MDR strains include: Vancomycin-Resistant Enterococci (VRE), Methicillin-Resistant Staphylococcus aureus (MRSA), Extended-spectrum β-lactamase (ESBLs) producing Gram-negative bacteria, Klebsiella pneumoniae carbapenemase (KPC) producing Gram-negatives, and Multidrug-Resistant gram negative rods (MDR GNR) MDRGN bacteria such as Enterobacter species E. coli, Klebsiella pneumoniae, Acinetobacter baumannii, or Pseudomonas aeruginosa.

Environmental Therapy

In some embodiments, bacteriophages disclosed herein are further used for food and agriculture sanitation (including meats, fruits and vegetable sanitation), hospital sanitation, home sanitation, vehicle and equipment sanitation, industrial sanitation, etc. In some embodiments, bacteriophages disclosed herein are used for the removal of antibiotic-resistant or other undesirable pathogens from medical, veterinary, animal husbandry, or any additional environments bacteria are passed to humans or animals.

Environmental applications of phage in health care institutions are for equipment such as endoscopes and environments such as ICUs which are potential sources of nosocomial infection due to pathogens that are difficult or impossible to disinfect. In some embodiments, a phage disclosed herein is used to treat equipment or environments inhabited by bacterial genera such as Pseudomonas which become resistant to commonly used disinfectants. In some embodiments, phage compositions disclosed herein are used to disinfect inanimate objects. In some embodiments, an environment disclosed herein is sprayed, painted, or poured onto with aqueous solutions with phage titers. In some embodiment a solution described herein comprises between 10¹-10²⁰ plaque forming units (PFU)/ml. In some embodiments, a bacteriophage disclosed herein is applied by aerosolizing agents that, in some embodiments, include dry dispersants to facilitate distribution of the bacteriophage into the environment. In some embodiments, objects are immersed in a solution containing bacteriophage disclosed herein.

Sanitation

In some embodiments, bacteriophages disclosed herein are used as sanitation agents in a variety of fields. Although the terms “phage” or “bacteriophage” may be used, it should be noted that, where appropriate, this term should be broadly construed to include a single bacteriophage, multiple bacteriophages, such as a bacteriophage mixtures and mixtures of a bacteriophage with an agent, such as a disinfectant, a detergent, a surfactant, water, etc.

In some embodiments, bacteriophages are used to sanitize hospital facilities, including operating rooms, patient rooms, waiting rooms, lab rooms, or other miscellaneous hospital equipment. In some embodiments, this equipment includes electrocardiographs, respirators, cardiovascular assist devices, intraaortic balloon pumps, infusion devices, other patient care devices, televisions, monitors, remote controls, telephones, beds, etc. In some situations, the bacteriophage is applied through an aerosol canister. In some embodiments, bacteriophage is applied by wiping the phage on the object with a transfer vehicle.

In some embodiments, a bacteriophage described herein is used in conjunction with patient care devices. In some embodiment, bacteriophage is used in conjunction with a conventional ventilator or respiratory therapy device to clean the internal and external surfaces between patients. Examples of ventilators include devices to support ventilation during surgery, devices to support ventilation of incapacitated patients, and similar equipment. In some embodiments, the conventional therapy includes automatic or motorized devices, or manual bag-type devices such as are commonly found in emergency rooms and ambulances. In some embodiments, respiratory therapy includes inhalers to introduce medications such as bronchodilators as commonly used with chronic obstructive pulmonary disease or asthma, or devices to maintain airway patency such as continuous positive airway pressure devices.

In some embodiments, a bacteriophage described herein is used to cleanse surfaces and treat colonized people in an area where highly-contagious bacterial diseases, such as meningitis or enteric infections are present.

In some embodiments, water supplies are treated with a composition disclosed herein. In some embodiments, bacteriophage disclosed herein is used to treat contaminated water, water found in cisterns, wells, reservoirs, holding tanks, aqueducts, conduits, and similar water distribution devices. In some embodiments, the bacteriophage is applied to industrial holding tanks where water, oil, cooling fluids, and other liquids accumulate in collection pools. In some embodiments, a bacteriophage disclosed herein is periodically introduced to the industrial holding tanks in order to reduce bacterial growth.

In some embodiments, bacteriophages disclosed herein are used to sanitize a living area, such as a house, apartment, condominium, dormitory, or any living area. In some embodiments, the bacteriophage is used to sanitize public areas, such as theaters, concert halls, museums, train stations, airports, pet areas, such as pet beds, or litter boxes. In this capacity, the bacteriophage is dispensed from conventional devices, including pump sprayers, aerosol containers, squirt bottles, pre-moistened towelettes, etc., applied directly to (e.g., sprayed onto) the area to be sanitized, or is transferred to the area via a transfer vehicle, such as a towel, sponge, etc. In some embodiments, a phage disclosed herein is applied to various rooms of a house, including the kitchen, bedrooms, bathrooms, garage, basement, etc. In some embodiments, a phage disclosed herein is in the same manner as conventional cleaners. In some embodiments, the phage is applied in conjunction with (before, after, or simultaneously with) conventional cleaners provided that the conventional cleaner is formulated so as to preserve adequate bacteriophage biologic activity.

In some embodiments, a bacteriophage disclosed herein is added to a component of paper products, either during processing or after completion of processing of the paper products. Paper products to which a bacteriophage disclosed herein is added include, but are not limited to, paper towels, toilet paper, moist paper wipes.

Food Safety

In some embodiments, a bacteriophage described herein is used in any food product or nutritional supplement, for preventing contamination. Examples for food or pharmaceuticals products are milk, yoghurt, curd, cheese, fermented milks, milk based fermented products, ice-creams, fermented cereal based products, milk based powders, infant formulae or tablets, liquid suspensions, dried oral supplement, wet oral supplement, or dry-tube-feeding.

The broad concept of bacteriophage sanitation is applicable to other agricultural applications and organisms. Produce, comprises fruits and vegetables, dairy products, and other agricultural products. For example, freshly-cut produce frequently arrive at the processing plant contaminated with pathogenic bacteria. This has led to outbreaks of food-borne illness traceable to produce. In some embodiments, the application of bacteriophage preparations to agricultural produce substantially reduce or eliminate the possibility of food-borne illness through application of a single phage or phage mixture with specificity toward species of bacteria associated with food-borne illness. In some embodiments, bacteriophages are applied at various stages of production and processing to reduce bacterial contamination at that point or to protect against contamination at subsequent points.

In some embodiments, specific bacteriophages are applied to produce in restaurants, grocery stores, produce distribution centers. In some embodiments, bacteriophages disclosed herein are periodically or continuously applied to the fruit and vegetable contents of a salad bar. In some embodiments, the application of bacteriophages to a salad bar or to sanitize the exterior of a food item is a misting or spraying process or a washing process.

In some embodiments, a bacteriophage described herein is used in matrices or support media containing with packaging containing meat, produce, cut fruits and vegetables, and other foodstuffs. In some embodiments, polymers that are suitable for packaging are impregnated with a bacteriophage preparation.

In some embodiments, a bacteriophage described herein is used in farm houses and livestock feed. In some embodiments, on a farm raising livestock, the livestock is provided with bacteriophage in their drinking water, food, or both. In some embodiments, a bacteriophage described herein is sprayed onto the carcasses and used to disinfect the slaughter area.

The use of specific bacteriophages as biocontrol agents on produce provides many advantages. For example, bacteriophages are natural, non-toxic products that will not disturb the ecological balance of the natural microflora in the way the common chemical sanitizers do, but will specifically lyse the targeted food-borne pathogens. Because bacteriophages, unlike chemical sanitizers, are natural products that evolve along with their host bacteria, new phages that are active against recently emerged, resistant bacteria, in some embodiments, are rapidly identified when required, whereas identification of a new effective sanitizer is a much longer process, several years.

### Pharmaceutical Compositions

In some embodiments, the disclosure provides pharmaceutical compositions and methods of administering the same to treat bacterial, archaeal infections or to disinfect an area. In some embodiments, the pharmaceutical composition comprises any of the reagents discussed above in a pharmaceutically acceptable carrier.

In some embodiments, compositions disclosed herein comprise medicinal agents, pharmaceutical agents, carriers, adjuvants, dispersing agents, diluents, and the like.

In some embodiments, the bacteriophages disclosed herein are formulated for administration in a pharmaceutical carrier in accordance with suitable methods. In some embodiments, the manufacture of a pharmaceutical composition according to the disclosure, the bacteriophage is admixed with, inter alia, an acceptable carrier. In some embodiments, the carrier is a solid (including a powder) or a liquid, or both, and is preferably formulated as a unit-dose composition. In some embodiments, one or more bacteriophages are incorporated in the compositions disclosed herein, which are prepared by any suitable method of a pharmacy.

In some embodiments, a method of treating subject's in-vivo, comprising administering to a subject a pharmaceutical composition comprising a bacteriophage disclosed herein in a pharmaceutically acceptable carrier, wherein the pharmaceutical composition is administered in a therapeutically effective amount. In some embodiments, the administration of the bacteriophage to a human subject or an animal in need thereof is by any means known in the art.

In some embodiments, bacteriophages disclosed herein are for oral administration. In some embodiments, the bacteriophages are administered in solid dosage forms, such as capsules, tablets, and powders, or in liquid dosage forms, such as elixirs, syrups, and suspensions. In some embodiments, compositions and methods suitable for buccal (sub-lingual) administration include lozenges comprising the bacteriophages in a flavored base, usually sucrose and acacia or tragacanth; and pastilles comprising the bacteriophages in an inert base such as gelatin and glycerin or sucrose and acacia.

In some embodiments, methods and compositions of the present disclosure are suitable for parenteral administration comprising sterile aqueous and non-aqueous injection solutions of the bacteriophage. In some embodiments, these preparations are isotonic with the blood of the intended recipient. In some embodiments, these preparations comprise antioxidants, buffers, bacteriostals and solutes which render the composition isotonic with the blood of the intended recipient. In some embodiments, aqueous and non-aqueous sterile suspensions include suspending agents and thickening agents. In some embodiments, compositions disclosed herein are presented in unit\dose or multi-dose containers, for example sealed ampoules and vials, and are stored in a freeze-dried (lyophilized) condition requiring only the addition of the sterile liquid carrier, for example, saline or water for injection on immediately prior to use.

In some embodiment, methods and compositions suitable for rectal administration are presented as unit dose suppositories. In some embodiments, these are prepared by admixing the bacteriophage with one or more conventional solid carriers, for example, cocoa butter, and then shaping the resulting mixture. In some embodiments, methods and compositions suitable for topical application to the skin are in the form of an ointment, cream, lotion, paste, gel, spray, aerosol, or oil. In some embodiments, carriers which are used include petroleum jelly, lanoline, polyethylene glycols, alcohols, transdermal enhancers, and combinations of two or more thereof.

In some embodiments, methods and compositions suitable for transdermal administration are presented as discrete patches adapted to remain in intimate contact with the epidermis of the recipient for a prolonged period of time.

In some embodiments, methods and compositions suitable for nasal administration or otherwise administered to the lungs of a subject include any suitable means, e.g., administered by an aerosol suspension of respirable particles comprising the bacteriophage compositions, which the subject inhales. In some embodiments, the respirable particles are liquid or solid. As used herein, “aerosol” includes any gas-borne suspended phase, which is capable of being inhaled into the bronchioles or nasal passages. In some embodiments, aerosols of liquid particles are produced by any suitable means, such as with a pressure-driven aerosol nebulizer or an ultrasonic nebulizer. In some embodiments, aerosols of solid particles comprising the composition are produced with any solid particulate medicament aerosol generator, by techniques known in the pharmaceutical art.

In some embodiment, methods and compositions suitable for administering bacteriophages disclosed herein to a surface of an object or subject includes aqueous solutions. In some embodiments, such aqueous solutions are sprayed onto the surface of an object or subject. In some embodiment, the aqueous solutions are used to irrigate and clean a physical wound of a subject form foreign debris including bacteria.

In some embodiments, the bacteriophages disclosed herein are administered to the subject in a therapeutically effective amount. In some embodiments, at least one bacteriophage composition disclosed herein is formulated as a pharmaceutical formulation. In some embodiments, a pharmaceutical formulation comprises 1, 2, 3, 4, 5, 6, 7, 8, 9, 10, 11, 12, 13, 14, 15, 16, 17, 18, 19, 20 or more bacteriophage disclosed herein. In some instances, a pharmaceutical formulation comprises a bacteriophage described herein and at least one of: an excipient, a diluent, or a carrier.

In some embodiments, a pharmaceutical formulation comprises an excipient. Excipients are described in the Handbook of Pharmaceutical Excipients, American Pharmaceutical Association (1986) and includes but are not limited to solvents, dispersion media, diluents, or other liquid vehicles, dispersion or suspension aids, surface active agents, isotonic agents, thickening or emulsifying agents, preservatives, solid binders, and lubricants.

Non-limiting examples of suitable excipients include but is not limited to a buffering agent, a preservative, a stabilizer, a binder, a compaction agent, a lubricant, a chelator, a dispersion enhancer, a disintegration agent, a flavoring agent, a sweetener, a coloring agent.

In some embodiments, an excipient is a buffering agent. Non-limiting examples of suitable buffering agents include but is not limited to sodium citrate, magnesium carbonate, magnesium bicarbonate, calcium carbonate, and calcium bicarbonate. In some embodiments, a pharmaceutical formulation comprises any one or more buffering agent listed: sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts.

In some embodiments an excipient is a preservative. Non-limiting examples of suitable preservatives include but is not limited to antioxidants, such as alpha-tocopherol and ascorbate, and antimicrobials, such as parabens, chlorobutanol, and phenol. In some embodiments, antioxidants include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some embodiments, preservatives include validamycin A, TL-3, sodium ortho vanadate, sodium fluoride, N-α-tosyl-Phe-chloromethylketone, N-α-tosyl-Lys-chloromethylketone, aprotinin, phenylmethylsulfonyl fluoride, diisopropylfluorophosphate, protease inhibitor, reducing agent, alkylating agent, antimicrobial agent, oxidase inhibitor, or other inhibitor.

In some embodiments, a pharmaceutical formulation comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments, the binders that are used in a pharmaceutical formulation are selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some embodiments, a pharmaceutical formulation comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethylene glycol, sodium benzoate, sodium lauryl sulfate, magnesium lauryl sulfate, and light mineral oil. In some embodiments, lubricants that are in a pharmaceutical formulation are selected from metallic stearates (such as magnesium stearate, calcium stearate, aluminium stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments, an excipient comprises a flavoring agent. In some embodiments, flavoring agents includes natural oils; extracts from plants, leaves, flowers, and fruits; and combinations thereof.

In some embodiments, an excipient comprises a sweetener. Non-limiting examples of suitable sweeteners include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like.

In some instances, a pharmaceutical formulation comprises a coloring agent. Non-limiting examples of suitable color agents include food, drug and cosmetic colors (FD&C), drug and cosmetic colors (D&C), and external drug and cosmetic colors (Ext. D&C).

In some embodiments, the pharmaceutical formulation disclosed herein comprises a chelator. In some embodiments, a chelator includes ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA.

In some instances, a pharmaceutical formulation comprises a diluent. Non-limiting examples of diluents include water, glycerol, methanol, ethanol, and other similar biocompatible diluents. In some embodiments, a diluent is an aqueous acid such as acetic acid, citric acid, maleic acid, hydrochloric acid, phosphoric acid, nitric acid, sulfuric acid, or similar.

In some embodiments, a pharmaceutical formulation comprises a surfactant. In some embodiments, surfactants are be selected from, but not limited to, polyoxyethylene sorbitan fatty acid esters (polysorbates), sodium lauryl sulphate, sodium stearyl fumarate, polyoxyethylene alkyl ethers, sorbitan fatty acid esters, polyethylene glycols (PEG), polyoxyethylene castor oil derivatives, docusate sodium, quaternary ammonium compounds, amino acids such as L-leucine, sugar esters of fatty acids, glycerides of fatty acids or a combination thereof.

In some instances, a pharmaceutical formulation comprises an additional pharmaceutical agent. In some embodiments, an additional pharmaceutical agent is an antibiotic agent. In some embodiments, an antibiotic agent is of the group consisting of aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides or tetracycline.

In some embodiments, an antibiotic agent described herein is an aminoglycoside such as Amikacin, Gentamicin, Kanamycin, Neomycin, Netilmicin, Tobramycin or Paromomycin. In some embodiments, an antibiotic agent described herein is an Ansamycin such as Geldanamycin or Herbimycin.

In some embodiments, an antibiotic agent described herein is a carbacephem such as Loracarbef. In some embodiments, an antibiotic agent described herein is a carbapenem such as Ertapenem, Doripenem, Imipenem/Cilastatin or Meropenem.

In some embodiments, an antibiotic agent described herein are cephalosporins (first generation) such as Cefadroxil, Cefazolin, Cefalexin, Cefalotin or Cefalothin, or alternatively a Cephalosporins (second generation) such as Cefaclor, Cefamandole, Cefoxitin, Cefprozil or Cefuroxime. In some embodiments, an antibiotic agent is a Cephalosporins (third generation) such as Cefixime, Cefdinir, Cefditoren, Cefoperazone, Cefotaxime, Cefpodoxime, Ceftibuten, Ceftizoxime and Ceftriaxone or a Cephalosporins (fourth generation) such as Cefepime or Ceftobiprole.

In some embodiments, an antibiotic agent described herein is a lincosamide such as Clindamycin and Azithromycin, or a macrolide such as Azithromycin, Clarithromycin, Dirithromycin, Erythromycin, Roxithromycin, Troleandomycin, Telithromycin and Spectinomycin.

In some embodiments, an antibiotic agent described herein is a monobactams such as Aztreonam, or a nitrofuran such as Furazolidone or Nitrofurantoin.

In some embodiments, an antibiotic agent described herein is a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin and Ticarcillin.

In some embodiments, an antibiotic agent described herein is a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, or Trimethoprim-Sulfamethoxazole (Co-trimoxazole) (TMP-SMX).

In some embodiments, an antibiotic agent described herein is a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin.

In some embodiments, an antibiotic agent described herein is a polypeptide such as Bacitracin, Colistin or Polymyxin B.

In some embodiments, an antibiotic agent described herein is a tetracycline such as Demeclocycline, Doxycycline, Minocycline or Oxytetracycline.

Dose

Dose and duration of the administration of a composition disclosed herein will depend on a variety of factors, including the subject's age, subject's weight, and tolerance of the phage. In some embodiments, a bacteriophage disclosed herein is administered to patients by oral administration. In some embodiments, a dose of phage between 10³ and 10²⁰ PFU is given. For example, in some embodiments, the bacteriophage is present in a composition in an amount between 10³ and 10¹¹PFU. In some embodiments, the bacteriophage is present in a composition in an amount about 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², 10²³, 10²⁴ PFU or more. In some embodiments, the bacteriophage is present in a composition in an amount of less than 10¹PFU. In some embodiments, the bacteriophage is present in a composition in an amount between 10¹ and 10⁸, 10⁴ and 10⁹, 10⁵ and 10¹⁰, or 10⁷ and 10¹¹PFU. In some embodiments, a bacteriophage or a mixture is administered to a subject in need thereof 1, 2, 3, 4, 5, 6, 7, 8, 9, 10, 11, 12, 13, 14, 15, 16, 17, 18, 19, 20, 21, 22, 23, or 24 times a day. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, or 21 times a week. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some embodiments, the compositions (bacteriophage) disclosed herein are administered before, during, or after the occurrence of a disease or condition. In some embodiment, the timing of administering the composition containing the bacteriophage varies. In some embodiments, the pharmaceutical compositions are used as a prophylactic and are administered continuously to subjects with a propensity to conditions or diseases in order to prevent the occurrence of the disease or condition. In some embodiments, pharmaceutical compositions are administered to a subject during or as soon as possible after the onset of the symptoms. In some embodiments, the administration of the compositions is initiated within the first 48 hours of the onset of the symptoms, within the first 24 hours of the onset of the symptoms, within the first 6 hours of the onset of the symptoms, or within 3 hours of the onset of the symptoms. In some embodiments, the initial administration of the composition is via any route practical, such as by any route described herein using any formulation described herein. In some embodiments, the compositions is administered as soon as is practicable after the onset of a disease or condition is detected or suspected, and for a length of time necessary for the treatment of the disease, such as, for example, from about 1 month to about 3 months. In some embodiments, the length of treatment will vary for each subject.

### Kits

Disclosed herein are kits for use. In some embodiments, the kit comprises the nucleic acid constructs for the CRISPR arrays, exogenous Cpf1, transcriptional activators, and/or anti-CRISPR polypeptides, as well as the bacteriophages and/or any other vectors/expression cassettes disclosed herein in a form suitable for introduction into a cell and/or administration to a subject. In some embodiments, the kit comprises other therapeutic agents, carriers, buffers, containers, devices for administration, and the like. In some embodiments, the kit comprises labels and/or instructions for repression of expression a target gene and/or modulation of repression of expression of a target gene. In some embodiments, labeling and/or instructions include, for example, information concerning the amount, frequency and method of introduction and/or administration of the nucleic acid constructs for the CRISPR arrays, exogenous Cpf1, transcriptional activators, and anti-CRISPR polypeptides, as well as the bacteriophages and/or any other vectors/expression cassettes.

In some embodiments, a kit for the killing of one target bacterium is provided, said kit comprising, consisting essentially of, consisting of nucleic acid constructs for the CRISPR arrays, exogenous Cpf1, transcriptional activators, and/or anti-CRISPR polypeptides, as well as the bacteriophages and/or any other vectors/expression cassettes necessary to achieve killing of the target bacteria by any embodiment disclosed herein.

In some embodiments, a kit is provided for modulating the activity of a CRISPR-Cpf1 system in a target bacterium is provided, the kit comprising, consisting essentially of, consisting of nucleic acid constructs for the CRISPR arrays, exogenous Cpf1, transcriptional activators, and anti-CRISPR polypeptides, as well as the bacteriophages and/or any other vectors/expression cassettes necessary to achieve modulation of a CRISPR-Cpf1 system in a target bacteria by any embodiment disclosed herein.

In some embodiments, the nucleic acid constructs for the CRISPR arrays, exogenous Cpf1, transcriptional activators, and/or anti-CRISPR polypeptides of said kits are comprised on a single vector or expression cassette or on separate vectors or expression cassettes or within a single bacteriophage or a plurality of bacteriophages. In some embodiments, a kit comprises one or more bacteriophage disclosed herein. In some embodiments, the kits comprise instructions for use. In some embodiments, the instructions for practicing the methods are recorded on a suitable recording medium. In some embodiments, the instructions are printed on a substrate, such as paper or plastic, etc. In some embodiments, the instructions are present in the kits as a package insert, in the labeling of the container of the kit or components thereof (i.e., associated with the packaging or subpackaging) etc. In some embodiments, the instructions are present as an electronic storage data file present on a suitable computer readable storage medium, e.g. CD-ROM, diskette, flash drive, etc. In some embodiments, the actual instructions are not present in the kit, but means for obtaining the instructions from a remote source (e.g. via the Internet), are provided. In some embodiments, the kit includes a web address where the instructions are viewed and/or from which the instructions are downloaded.

Certain embodiments disclosed herein, both in their methods and compositions, now are described with reference to the following examples. It should be appreciated that these examples are not intended to limit the scope of the claims to the disclosure, but are rather intended to be exemplary of certain embodiments. Any variations in the exemplified methods that occur to the skilled artisan are intended to fall within the scope of the disclosure.

### EXAMPLES

### Example 1: Overview for Generating CRISPR Enhanced Bacteriophages

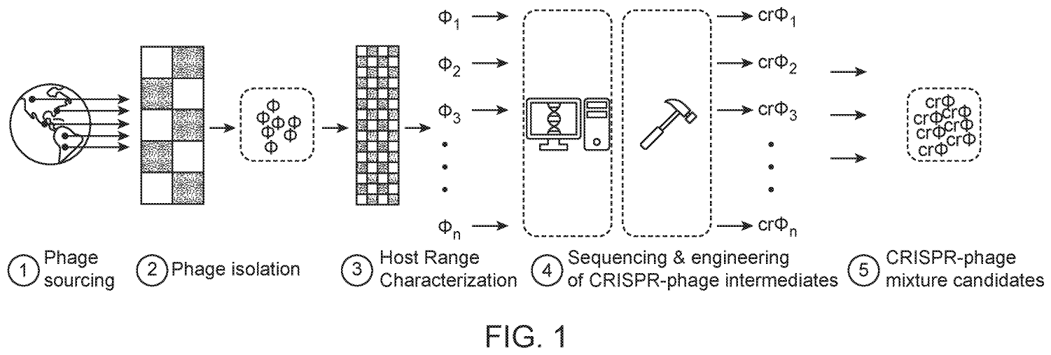

CRISPR-enhanced bacteriophages are phages that have been engineered to express CRISPR RNA constructs from a bacteriophage genome that maintains the essential genes for lytic lifestyle. The steps involved are sourcing, isolating and identifying bacteriophages and cocktails of bacteriophages with broad host ranges against bacteria followed by engineering each phage to carry an expression construct (for example, crRNA) that targets the bacterium's genome and validating optimized combinations of crPhages to be used as a clinical lead candidate. In some embodiments, the general process is as schematically shown in steps 1-5 of FIG. 1. Steps 1-5 are designed to identify a suitable number of wild-type bacteriophages such that they:

1. Meet minimum quality standards (absence of lysogeny, virulence genes or antibiotic resistance genes) proposed in Table 1 below:

TABLE 1
Summary of phage characterizations

| Test/characteristic | Method |
|---|---|
| Genome size (kb) | Genome sequencing |
| Family of Caudovirales | Transmission electron microscopy |
| Host range activity | Host range analysis against uropathogenic |
| | E. coli clinical isolates and representative |
| | E. coli strains |
| Genome sequence | Genome sequencing |
| DNA restriction profile | Restriction enzyme digestion/electrophoresis |
| Typing | PCR specific to engineered insert |
| Lifestyle (lytic, temperate) | DNA analysis |
| Absence of generalized | Microbiological transduction assay |
| transduction | |
| Absence of virulence genes | Genome sequence analysis |
| Absence of antibiotic | Genome sequence analysis |
| resistance genes | |

2. Have collective activity against approximately 90% or greater of the clinical isolate panel.

3. Result in infection of each strain by at least 2 phages within the cocktail (mixture of two or more phages), intended to ensure strain sensitivity to the cocktail in the event of resistance to any single bacteriophage. In some embodiments, a cocktail described herein in comprises at least 2, 3, 4, 5, 6, 7, 8, 9, 10, 20, 30, 50, 100 or more bacteriophages.

4. Include genetic engineering of each candidate bacteriophage to express a crRNA construct from the wild-type genome. Each engineered crPhage is intended to retain lytic activity. crPhages are subjected to in vitro analyses to assess host range and in vitro efficacy. These studies are intended to confirm that crPhages retain broad host range, if not expanded host range by ability to transduce lethal crRNA constructs in the absence of productive lytic infection, and improved lethality for each crPhage to the cognate wild-type bacteriophage.

5. Identifying crPhages for use in a cocktail that is optimized to improve host range, manufacturing limitations or nonclinical efficacy.

### Example 2: Bacterial Strains and Growth Conditions

Bacterial strains and growth conditions. Bacterial strains used in the examples describe herein were Escherichia coli MG1655, E. coli BW25113, E. coli O9:HS, Enterohemorrhagic E. coli (ETEC) E2437A, Shigella dysenteriae serotype 1 ATCC9361, Klebsiella pneumoniae subsp. pneumoniae ATCC700721, and Salmonella enterica LT2. E. coli TOP10 and E. coli NEB was used for cloning and vector construction and were cultured in Luria-Bertani medium supplemented with antibiotics when appropriate (50 μg/mL kanamycin, 100 μg/mL Ampicillin and 25 μg/mL Chloramphenicol). All E. coli, Shigella dysenteriae, and Salmonella strains were cultured in LB medium (10 g/liter tryptone, 5 g/liter yeast extract, and 10 g/liter sodium chloride) at 37° C. and 250 rpm with appropriate antibiotics. The same strains were plated on LB agar (LB medium with 1.5% agar) supplemented with appropriate inducers and incubated at 37° C. K. pneumoniae was cultured in LB medium (10 g/liter tryptone, 5 g/liter yeast extract, and 10 g/liter sodium chloride) supplemented with 0.5% EDTA (ref.).

### Example 3: Plasmid Construction and Cloning

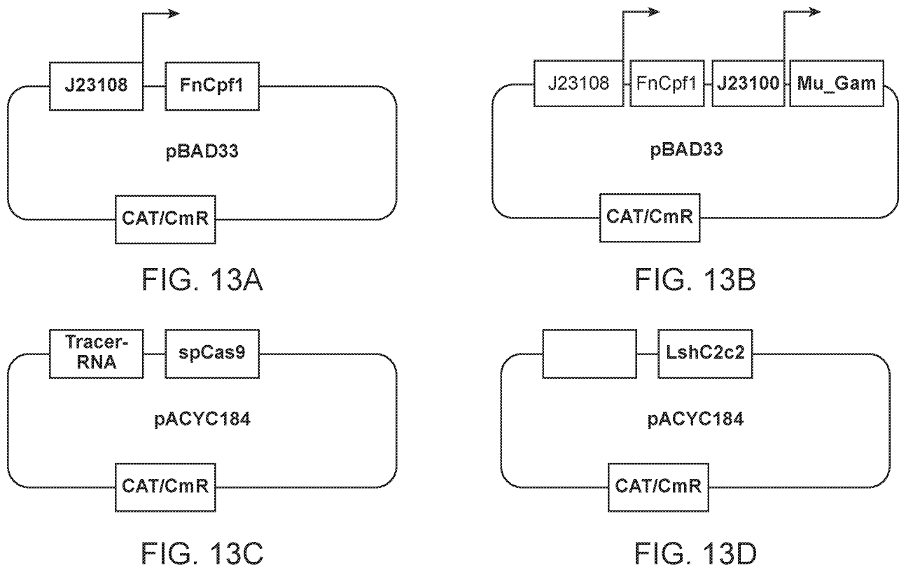

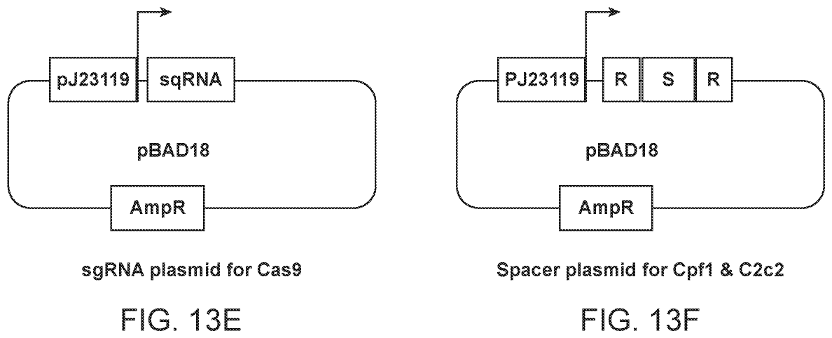

For pBAD33-Cas12a, the Cpf1 (Cas12a) gene from Francisella novicida U112 was generated by PCR amplification using a template of pFnCpf1 (Addgene #69973) and ligated into pBAD33 with constitutive promoter J23108 upstream of the Cpf1 (Cas12a) gene (FIG. 13A). To generate the pBAD33-Cpf1-MuGam plasmid, Mu gam sequence was generated by a chemically synthesized gBlock (IDT) and then inserted by Gibson assembly into pBAD33-Cpf1 (FIG. 13B). To generate the sgRNA for the SpyCas9, an sgRNA with a constitutive promoter was cloned into a plasmid. To generate spacers for Cas12a (Cpf1) and Cas13a (C2c2), golden gate assembly was used. All site-directed mutagenesis for Cpf1 (Cas12a) and Cas13a was done with Q5® Site-Directed Mutagenesis Kit (NEB). The guide or CRISPR array spacer was designed to be a length of 20 nts (for Cas9), 25 nts (for Cpf1 (Cas12a)), or 26 nt (for Cas13a) and in target genes of interest. To identify the specific sequence, PAMs in the target genes were first identified. The genome sequence was then searched to determine whether the identified sequences were unique. Schematics of the plasmids comprising the sgRNA or spacer constructs are shown in FIG. 13.

### Example 4: Mutant Construction

In order to generate a recA deletion in E. coli MG1655 and S. enterica LT2, temperature sensitive pKD46 plasmids were transformed into recipient strain E. coli MG1655 or S. enterica LT2. Cells were recovered and incubated at 30° C. overnight. The transformed colonies containing pKD46 plasmid with recipient strain E. coli MG1655 or S. enterica LT2 were used for recombineering. A homologous recombination (HR) cassette with pKD13 plasmid (KanR) was prepared by PCR amplification, the forward and reverse primer have 50-bp homology arms beside recA followed by FRT-flanked resistance cassette. The PCR product was digested with DpnI and column purification and ready for recombineering in a strain with pKD46. A single colony of the recipient strain containing pKD46 was inoculated in 3 ml LB medium containing ampicillin, grown at 30° C. for overnight. The following day, 100 ul of the overnight culture was back diluted in 25 ml LB containing ampicillin and 0.2% arabinose. Arabinose induces the expression of the recombinase. This mixture was incubated at 30° C. to an OD600 of 0.6. Electrocompetent cells were prepared and electroporated using 50 ul cells and 10-1000 ng DNA of the PCR product prepared for recombineering. A negative cells-only control was also included. Cells with 500 ul SOC were recovered and incubated at 37° C. for 3-4 h. The higher temperature removed the pKD46 plasmid from the recovered cells, which were then plated 250 ul on Kanamycin (pKD13) at 37° C. After primary selection, replicate plate single colonies were made on Kanamycin plates and Ampicillin plates. Cells were expected to grow on Kanamycin and not in Ampicillin. Colonies only able to grow on the kanamycin plates were picked, DNA was isolated, and PCR was carried out to confirm gene knockout.

### Example 5: Transformation Assay

The pCas9, pBAD33-Cpf or pZ003(Cas13a) plasmids (with different sgRNA or spacers plasmids) were transformed into recipient E. coli strains, Shigella dysenteriae, Klebsiella pneumoniae and Salmonella enterica LT2 by electro transformation using 50 ng of plasmid DNA with a MicroPulser electroporator (Bio-Rad), and recovered in 450 ul of SOC medium for 1 h at 37° C. at 250 rpm. After the recovery period, cells were plated or spotted on serial dilutions on the appropriate selective media with antibiotics. CFUs were compared by the number of CFUs obtained with a control (Non-target sgRNA or Spacer) transformation. All transformations were repeated at least more than three times. For getting high killing efficiency, the sgRNA or spacer plasmid was transformed and purified in its own native host (Shigella dysenteriae, Klebsiella pneumoniae and Salmonella enterica). Then a killing assay was performed with the purified spacer plasmid.

### Example 6: Confirmation and Analysis of Escape

Colonies surviving the killing assay with the treF or yfaP spacer plasmid were re-streaked into LB agar plate with appropriate antibiotics. On the following day, single colonies were inoculated in 3 ml LB medium with antibiotics for overnight and growth was assessed based on the optical density A600. Cultures exhibiting measurable growth for non-target spacer was (A600 1.0-2.0), for treF (A600 0.1-0.4) and for yfaP (A600 0.1-0.3). Thereafter, plasmids were isolated from the same culture and sent for direct sequencing or PCR was performed to amplify spacers (tref or yfaP) for confirmation of a missing or mutated spacer. Primers that were used for spacers (treF & yfaP) specifically bound within the promoter or the terminator of the spacer plasmid. All data were statistically analyzed using GraphPad Prism version 7.0 (GraphPad Software, San Diego, Calif., USA, http://www.graphpad.com/).

### Example 7: Comparison of Antimicrobial Activity of Cpf1 (Cas12a) Over Cas9 and Cas13a

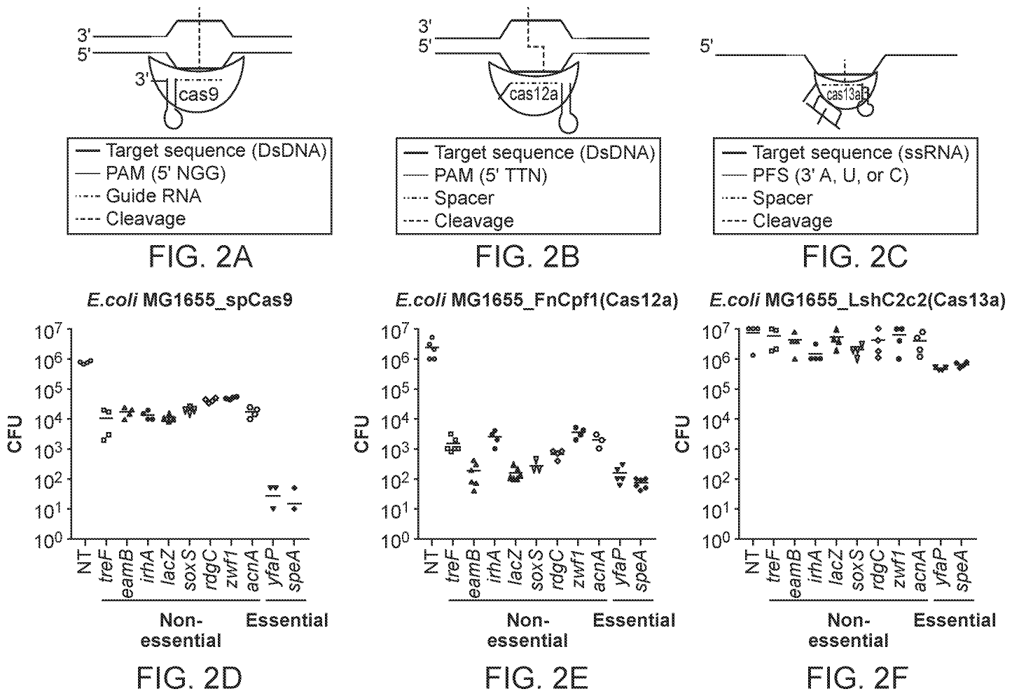

Cpf1 (Cas12a) was explored as an alternative effector for a CRISPR-Cas based antimicrobial as compared to Cas9 and Cas13a (FIG. 2A-FIG. 2C). Cas13a is also referred to as C2c2 herein. A plasmid based CRISPR array (sgRNA or spacer) was designed to target 10 genes throughout the E. coli MG1655 genome, of these 10 genes eight were non-essential (treF, eamB, irhA, lacZ, soxS, rdgC, zwfl and acnA) and two were essential (yfaP and speA). When Cas9 was used as the effector, the non-essential gene target killing efficiency increased by 10³-fold while essential gene killing efficiency increased by 10⁶ fold in comparison with non-target sgRNA (FIG. 2D). When Cpf1 (Cas12a) was used as the effector, both the non-essential and essential gene target killing efficiencies increased by 10³-10⁵-fold in comparison with the non-target spacer (FIG. 2E). None of the non-essential or essential genes showed any increase in the killing efficiency when Cas13a was used as the effector (FIG. 2F), which illustrated that Cas13a is not as good an antimicrobial in comparison to Cas9 or Cpf1 (Cas12a). These results indicated that among these three endonuclease, Cpf1 (Cas12a) is a better antimicrobial which showed a similar increase in killing efficiency regardless of whether a non-essential or essential gene was targeted. In contrast, when Cas9 was used as the effector, targeted the essential gene showed a greater killing efficiency increase in comparison to non-essential genes.

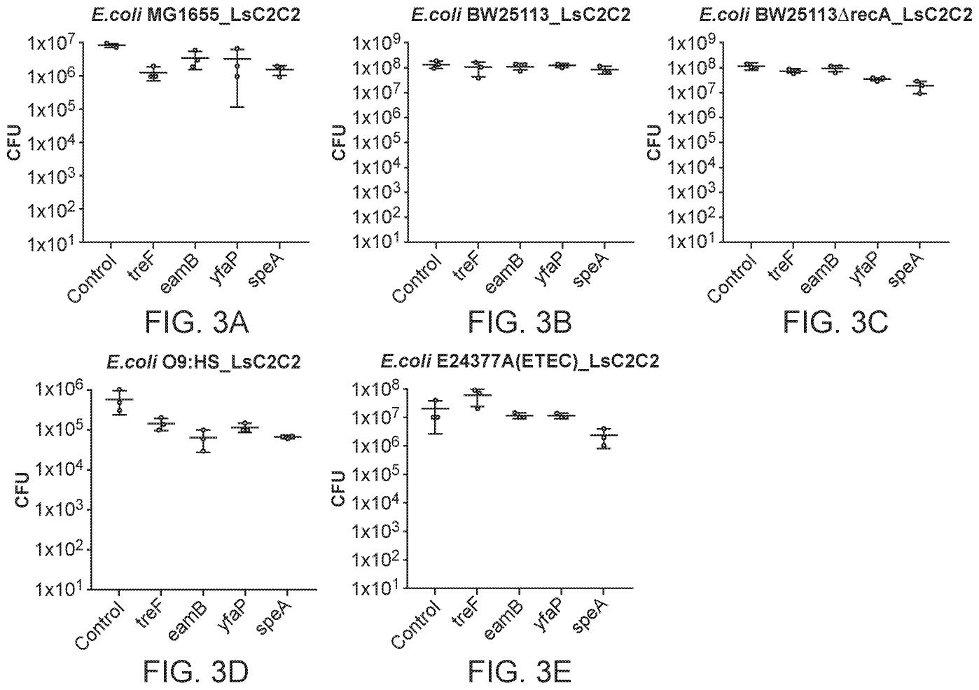

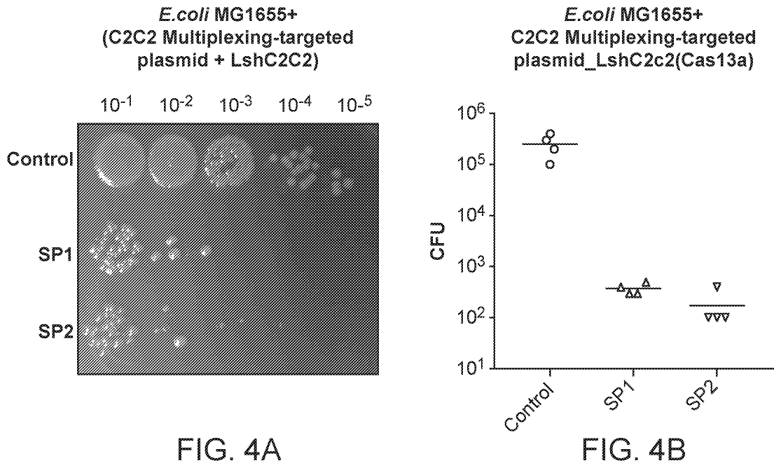

Cas13a mediated killing was further explored in other E. coli host such as E. coli BW25113, E. coli BW25113ΔrecA, E. coli O9:HS, E. coli E24377A by targeting two non-essential genes (treF and eamB) and two essential genes (yfaP and speA) but the results were consistent among all E. coli hosts (FIG. 3A-FIG. 3E). For a positive control, the multiplexing plasmid was targeted with two spacers, SP1 or SP2. SP1 and SP2 target killing efficiency increased by approximately 10⁴ fold (FIG. 4A-FIG. 4B).

### Example 8: Investigation of Escape Mechanisms of Cpf1 (Cas12a) and Cas9

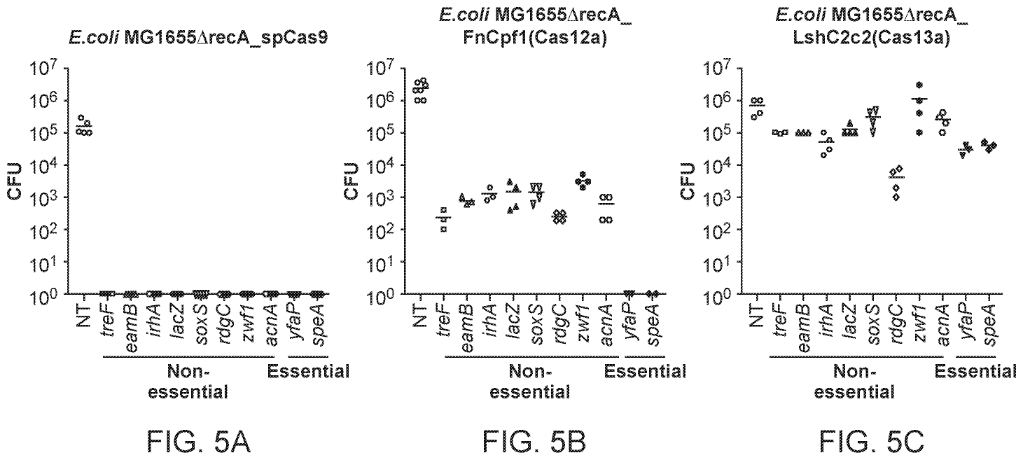

The differences in killing efficiency of Cpf1 (Cas12a) and Cas13a and their repair mechanisms was compared to Cas9, where the repair mechanism is controlled by recA. The killing efficiency of each endonuclease to transform essential and non-essential target genes in the E. coli MG1655ΔrecA genome is shown in FIG. 5A-FIG. 5C. In E. coli cells expressing Cas9 not a single cell survived (FIG. 5A), which illustrated that genome cleavage by Cas9 is repaired by recA. However, in E. coli cells expressing Cpf1 (Cas12a), there was not an effect on non-essential genes while essential genes showed an increase in killing efficiency and no cells surviving, same as Cas9 (FIG. 5B), indicating that for Cpf1 the repair mechanism of non-essential genes does not only depend on recA and other repair mechanisms exist. In E. coli cells expressing Cas13a, targeting rdgC showed a 10³-fold increase in killing efficiency compared to other target spacer and non-target spacer (FIG. 5C). RdgC protein is a potential negative regulator of RecA function and in certain embodiments, inhibits DNA strand exchange catalyzed by RecA filaments formed on single-stranded DNA by binding to the homologous duplex DNA and thereby blocking access to that DNA by the RecA nucleoprotein filaments. In certain embodiments, RdgC also binds non-specifically to single-stranded (ss) DNA and double-stranded DNA and degrades other non-target mRNA or ssDNA.

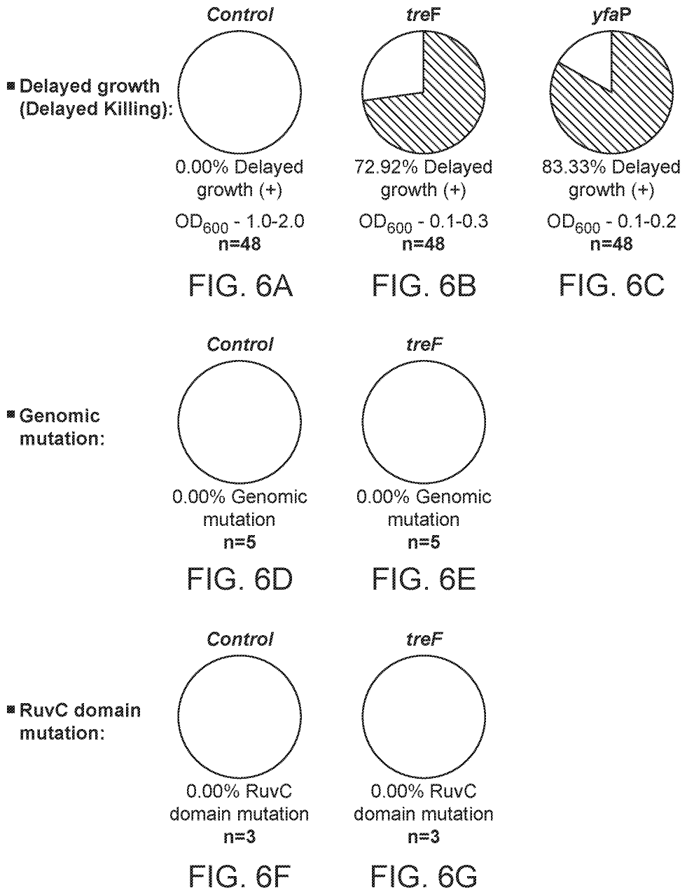

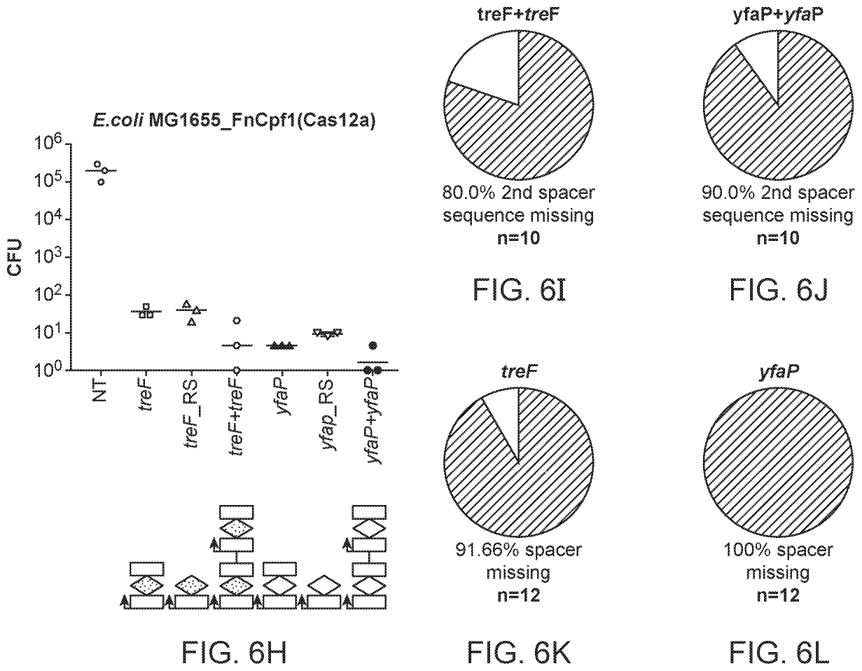

Growth patterns of 48 survivor colonies targeted by treF and yfaP spacers with Cpf1 (Cas12a) were investigated. Approximately 73-82% of the colonies showed delayed growth (FIG. 6A-FIG. 6C) in comparison to a non-target spacer, which showed that delayed killing of cells persisted after gene degradation. Genomic and plasmid mutation of treF and yfaP was also tested, as well as Cpf1 (Cas12a) RuvC mutation in same survivor colonies. No genomic mutations were found in cells transformed with either the non-target CRISPR array or the treF CRISPR array (FIG. 6D-FIG. 6E). Similarly, no mutations were found in the RuvC domain of plasmids isolated from the survivor colonies of either cells transformed with either the non-target CRISPR array or the treF CRISPR array (FIG. 6F-FIG. 6G). It was thought that deleterious mutation anywhere in the Cpf1 (Cas12a) protein disrupts targeting; the entire Cpf1 (Cas12a) protein was therefore sequenced after cell survival, but no change in sequence was observed. Additionally, the CRISPR array encoding the guide RNA was tested to see if it had undergone mutation. 12 colonies of treF and yfaP survivors that showed no growth defect were screened, and it was found that 11/12 and 12/12 colonies harbored CRISPR plasmids in which the array had undergone mutation.

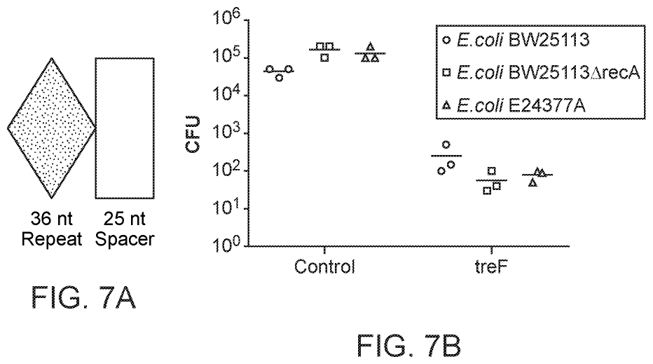

To further explore how cells were able to survive, a CRISPR array having double spacer plasmid was used in a killing assay in E. coli MG1655. Killing efficiency of CRISPR arrays having double spacer plasmids compared to single spacer plasmids is shown in FIG. 611. Out of 10 sequenced colonies, a first spacer was unchanged while a second spacer in approximately 8-9 of the sequenced colonies was missing (FIG. 6I-FIG. 6L) whereas cells having only a repeat and spacer CRISPR array did not show a change in spacer sequence and same as repeat-spacer-repeat CRISPR array (FIG. 7A-FIG. 7B). It was speculated that in CRISPR arrays having a double spacer, one spacer takes a role in targeting the genome and cleavage the genomic region targeted while another spacer plays a role in recombination or targeting of some other part of genome to enhance killing, but the exact mechanism is still a mystery. Overall cells survival by Cpf1 (Cas12a) is not controlled by one mechanism, but is controlled by various methods such as, for example, delayed growth or a missing spacer sequence.

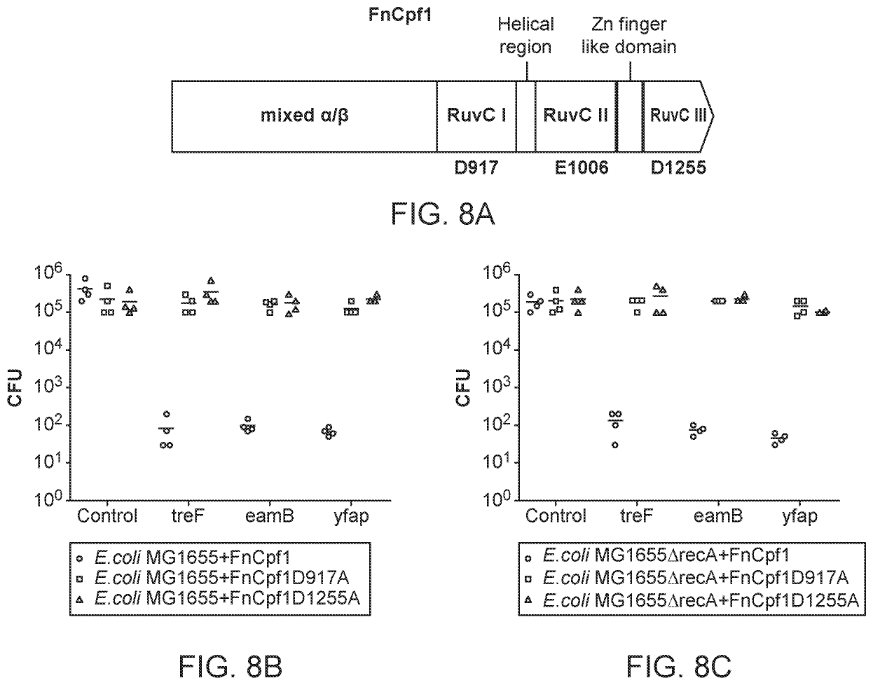

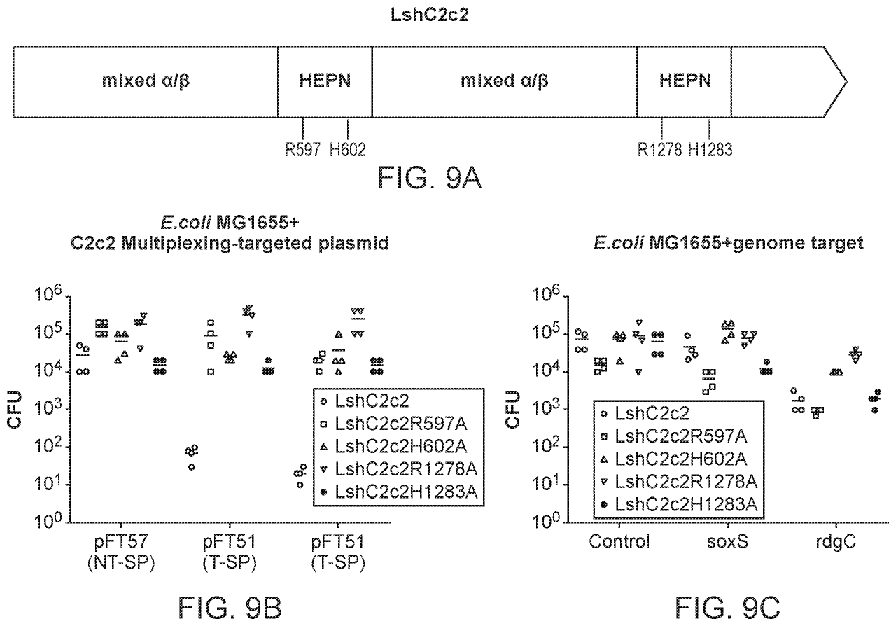

It has previously been shown that Cpf1 (Cas12a) has the ability to unspecifically degrade RNA, a mechanism controlled by a catalytic residue domain of Cpf1 (Cas12a). It was speculated that because of this phenomenon, Cpf1 (Cas12a) have an improved killing efficiency over Cas9. To investigate this, the D917 and D1255 domains of Cpf1 (Cas12a) were mutated (FIG. 8A) and killing experiments were performed in E. coli MG1655 and an E, coli MG1655 recA mutant by targeting treF, eamB and yfaP using CRISPR arrays. However, no effect in killing efficiency in the Cpf1 (Cas12a) wild type strain and RuvII domain strain was observed (FIG. 8B-FIG. 8C). The results showed that better killing efficiency was not dependent on the Cpf1 (Cas12a) catalytic domain. A similar experimental approach was also carried out for Cas13a, where a HEPN domain was mutated at R597, H602, R1278, and H1283 (FIG. 9A) and a killing experiment in E. coli MG1655 was performed by targeting SP1 or SP2 for the plasmid target (FIG. 9B) and soxS and rdgC for the genome target (FIG. 9C).

### Example 9: Cpf1 (Cas12a) Mediated Killing in Other E. coli Strains and Gram Negative Bacterial Pathogens

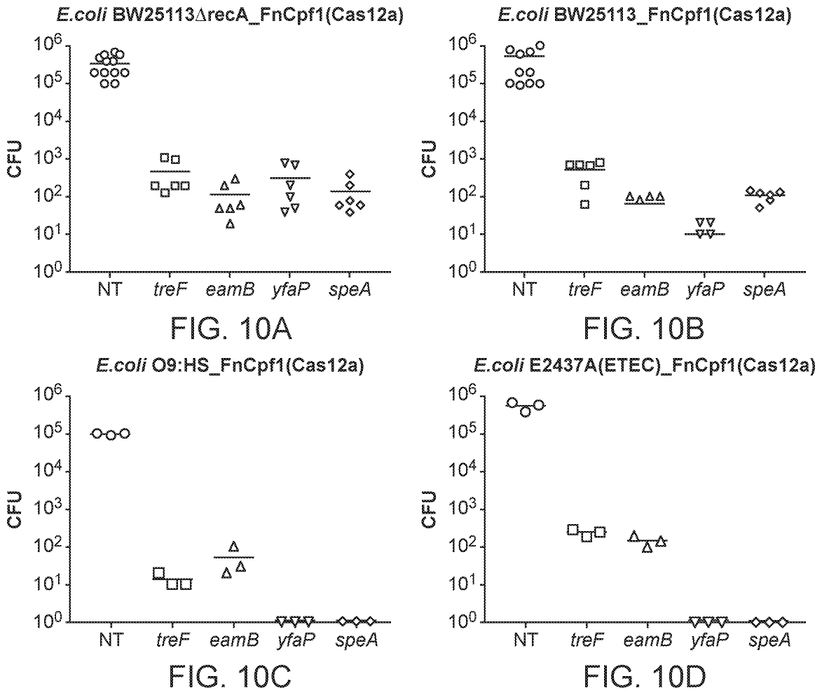

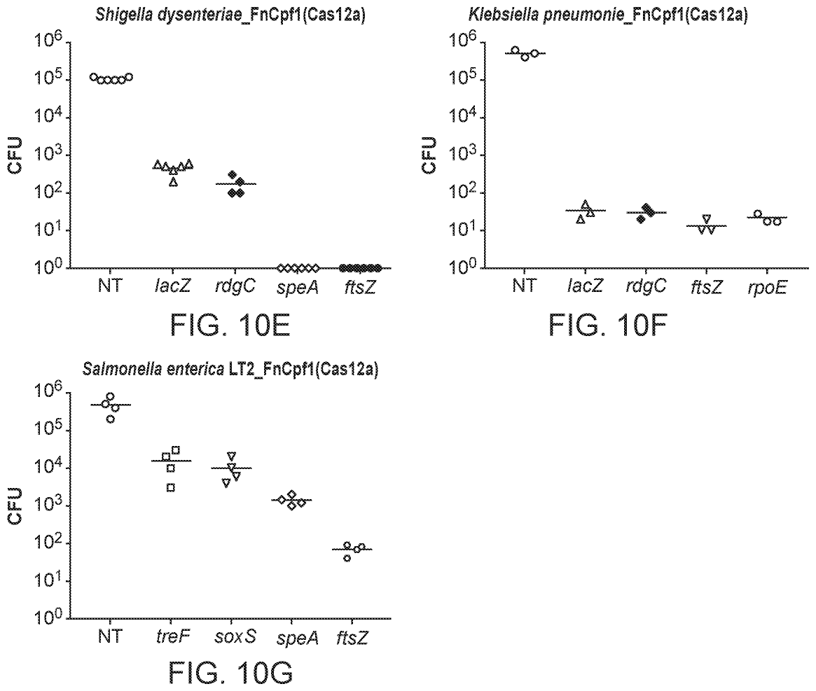

The utility of the Cpf1 (Cas12a) antimicrobial approach against other gram negative bacterial hosts, such as E. coli BW25113, E. coli O9:HS, E. coli E2437A(ETEC), Shigella dysenteriae, Klebsiella pneumoniae, and Salmonella enterica was demonstrated. For E. coli BW25113, E. coli BW25113ΔrecA, E. coli O9:HS, E. coli E2437A(ETEC) strains, CRISPR arrays targeting treF and eamB (non-essential) and speA and yfaP (essential) were designed. The killing efficiency in E. coli BW25113 strain (FIG. 10A-FIG. 10B) was consistent with E. coli MG1655 (FIG. 2D-FIG. 2F) for both non-essential and essential genes whereas in E. coli O9:HS (FIG. 10C) and E. coli E2437A(ETEC) (FIG. 10D) strains, killing completely increased in essential gene and is consistent with E. coli MG1655 for the non-essential genes targeted. The results showed that the repair mechanism of different species can vary even if the species are of the same genus.

Other gram negative bacterial pathogens were also investigated. For Shigella dysenteriae, a CRISPR array was used to target lacZ and rdgC (non-essential) and speA and ftsZ (essential). For Klebsiella pneumoniae, a CRISPR array was used to target lacZ and rdgC (non-essential) and rpoE and ftsZ (essential). For Salmonella enterica, a CRISPR array was used to target treF and soxS (non-essential) and speA and ftsZ (essential). The killing efficiency in Shigella dysenteriae increased for essential genes whereas the killing efficiency for non-essential genes increased by 10³ fold (FIG. 10E). Killing efficiency in Klebsiella pneumoniae by both non-essential and essential genes increased by 10⁴ fold (FIG. 10F), which showed it did not matter which gene was targeted (either non-essential or essential) in order to kill the cell. In case of Salmonella enterica, cell killing target by non-essential genes is very poor in comparison to E. coli strains and killing efficiency increased by 10²-10³ folds, while target by essential gene show 10³-10⁴ fold (FIG. 10G).

One repair mechanism did not work for all strains. There was also variation between genus as well as strains.

### Example 10: Enhancing Killing of Salmonella enterica LT2 by Mu Gam & Multiplex Spacer

Killing efficiency of a Salmonella enterica strain having a recA mutation with a CRISPR array targeting treF and ftsZ was determined. The killing efficiency increased by 10⁴ fold (FIG. 11B) in comparison to a non-target spacer, where as a wild type strain showed an increase in killing efficiency of only 10³ fold (FIG. 10G). These data represented Salmonella survival colonies were repaired by recA.

In order to make an efficient antimicrobial using Cpf1 (Cas12a) in Salmonella enterica, without creating any mutation in the wild type strain, a plasmid vector having Cpf1 (Cas12a) and Gam with a constitutive promoter to express Gam along with Cpf1 (Cas12a) was constructed. Gam is bacteriophage protein from Mu phage. It binds to a DNA double stranded break where recA is bound and inhibits functionality of recA to enhance killing efficiency (FIG. 11A). The Salmonella enterica strain having Cpf1 (Cas12a) and Gam showed an increase in killing efficiency of 10⁴-fold, similar to a recA mutant Salmonella (FIG. 11B). These data showed that the exogenous protein Gam enhanced the killing efficiency and did not require any genome modification in the parent strain to increase this efficiency.

Plasmid based CRISPR arrays to target treF gene in either four random locations in an individual spacer or all four spacers in the same plasmid (referred to as a multiplex spacer) were created (FIG. 11C). Killing experiments were then performed with these plasmids in a Salmonella wild type strain having Cpf1 (Cas12a) protein or Cpf1 (Cas12a) along with Gam. In a strain having Cpf1 (Cas12a) and a gene targeted by an individual spacer or a multiplex spacer, the killing efficiency increased by 10-10² fold (FIG. 11D). The result was unexpected, as it was expected that targeting the same gene in a different location is more lethal than single spacer. With a Salmonella wild type strain having Cpf1 (Cas12a) along with Gam and targeted by an individual spacer or multiplex spacer, it was found that an individual spacer had robust killing efficiency and killing efficiency was increased by 10⁴ fold (FIG. 11E), and more interestingly by targeting with multiplex spacer not a single colony survived.

### Example 11: Plasmid Expressed CPFI and Self-Targeting crRNAs Elicit Robust Cell Death

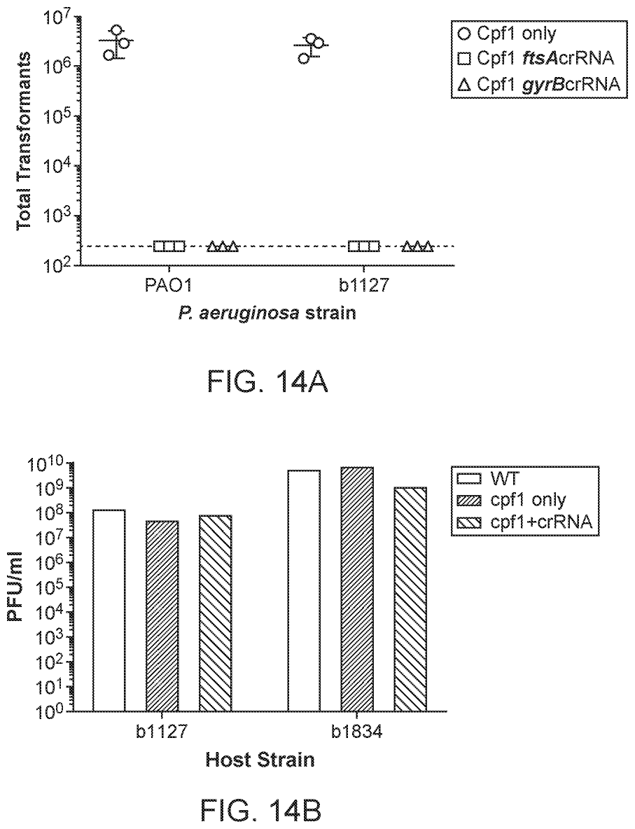

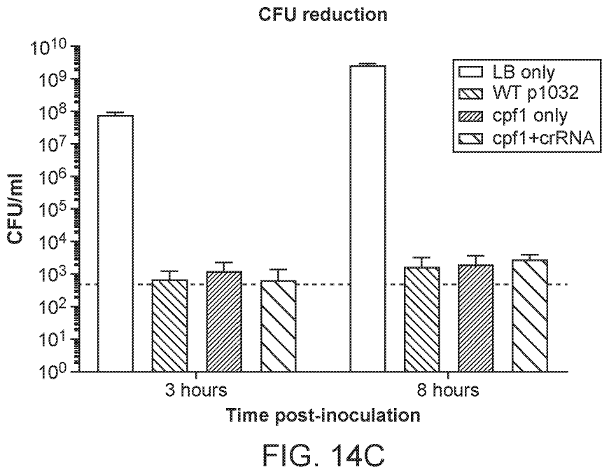

Plasmids encoding Cpf1 nuclease alone, Cpf1 nuclease and ftsA-targeting crRNA, or Cpf1 nuclease and gyrB-targeting crRNA were transformed into electrocompetent Pseudomonas aeruginosa. Bacteria were plated on carbenicillin containing plates to determine presence of the plasmid. While transformation of the control Cpf1 plasmid resulted in >10⁶ CFU per transformation, no carbenicillin-resistant colonies were recovered for the plasmids with crRNAs targeting Pseudomonas aeruginosa (FIG. 14A). Plasmid transformation of CPFI+crRNA illustrates its bacterial genome targeting lethality and utility as a nuclease for phage-delivered anti-microbial activity in two different Pseudomonas aeruginosa strains.

Two Pseudomonas strains (b1127 and b1843 were infected with wild-type (WT) phage, phage with Cpf1 inserted, or phage with Cpf1 and ftsA-targeting crRNA. These were grown for 16 hours, the bacteria were removed by filtration, and the concentration of phage (PFU/ml) was determined by plaquing dilutions of the phage onto 0.75% agar overlays containing the bacterial strain they were grown in. Phage p1032 and its CPFI engineered variants were assessed for their ability to amplify and demonstrated that the CPFI and CPFI+crRNA variants exhibited the same fitness in terms of final titer amplification as the wild-type counterpart on two different Pseudomonas aeruginosa strains. (FIG. 14B).

p1032 and its engineered variants were incubated with a susceptible Pseudomonas aeruginosa strain (b1127) and sampled at various times to enumerate bacterial cfus. At both 3 and 8 hours, the bacterial cfus are equivalent across the wild-type and engineered variants (FIG. 14C).

### Example 12: Growth Curve Host Range Analysis for WT and Engineered p1106

Table 2. illustrates growth curve host range analysis for wild-type Pseudomonas aeruginosa phage, Cpf1 encoding P. aeruginosa phage and Cpf1+crRNA encoding P. aeruginosa phage. Host range hits are defined by having a relative area under the growth curve of 0.7 or less. Briefly, Phage p1106 and its CPFI engineered variants were co-incubated with a subset of Pseudomonas aeruginosa strains and the optical density at 600 nm was monitored. The area under each growth curve was quantified and then divided by the area under each growth curve for an untreated culture for each strain. The values displayed represent the relative area under the curve and values <0.7 are considered within the host range of the phage. The host range of wild-type p1106 and its engineered variants were similar, demonstrating that the fitness of the phage in terms of strains it infects was unaltered by the insert of the CPFI and a crRNA.

TABLE 2

| Phage | p1106WT | p1106cpf1 | p1106FC |
|---|---|---|---|
| PA14 | 0.19 | 0.23 | 0.30 |
| 910 | 1.02 | 1.00 | 0.99 |
| PA14ΔCas | 0.23 | 0.24 | 0.45 |
| 1154 | 1.33 | 0.98 | 0.77 |
| 1161 | 1.28 | 1.00 | 0.91 |
| 1170 | 0.17 | 0.16 | 0.14 |
| PAO1 | 1.02 | 0.93 | 0.97 |
| 1284 | 0.97 | 0.89 | 0.98 |
| 919 | 0.91 | 0.75 | 0.89 |
| 1177 | 0.98 | 0.82 | 1.14 |
| 1162 | 0.99 | 0.76 | 0.98 |
| 1171 | 0.95 | 0.21 | 1.14 |
| 904 | 1.01 | 0.73 | 1.04 |
| 1285 | 0.96 | 0.74 | 1.13 |
| 920 | 0.90 | 0.87 | 0.86 |
| 1155 | 0.09 | 0.10 | 0.10 |
| 1163 | 0.10 | 0.10 | 0.09 |
| 1172 | 0.28 | 0.48 | 0.65 |
| 905 | 8.74 | 1.01 | 1.02 |
| 1286 | 0.28 | 0.24 | 0.22 |
| 903 | 0.99 | 0.99 | 1.00 |
| 1156 | 0.99 | 0.99 | 1.04 |
| 1165 | 0.96 | 1.02 | 1.27 |
| 1173 | 0.92 | 0.81 | 1.12 |
| 906 | 0.38 | 0.36 | 0.36 |
| 1287 | 1.00 | 0.91 | 0.95 |
| 922 | 0.99 | 1.07 | 0.80 |
| 1157 | 1.05 | 1.01 | 1.05 |
| 1166 | 0.97 | 1.08 | 1.22 |
| 1174 | 0.75 | 0.55 | 0.65 |
| 907 | 0.55 | 0.55 | 0.49 |
| 1288 | 1.12 | 0.91 | 1.02 |
| 923 | 1.29 | 0.89 | 0.90 |
| 1158 | 1.31 | 0.96 | 1.02 |
| 1167 | 1.29 | 0.97 | 1.00 |
| 1175 | 1.22 | 1.16 | 0.89 |
| 908 | 0.80 | 0.73 | 0.76 |
| 916 | 0.96 | 0.96 | 0.98 |
| 847 | 1.19 | 1.12 | 0.98 |
| 1159 | 0.99 | 0.92 | 1.06 |
| 1168 | 0.88 | 0.87 | 0.99 |
| 1176 | 0.79 | 0.81 | 0.84 |
| 909 | 0.74 | 0.66 | 0.71 |
| 917 | 0.77 | 0.76 | 0.80 |
| 805 | 1.02 | 0.99 | 1.02 |
| 1160 | 0.15 | 0.14 | 0.14 |
| 1169 | 0.16 | 0.14 | 0.15 |
| blank | 0.99 | 0.98 | 0.99 |
| % hit | 0.234042553 | 0.29787234 | 0.255319149 |

### Example 13: p1106 CFU Reduction Assays for PA14 Show Slightly Reduced Kill for Engineered Phages

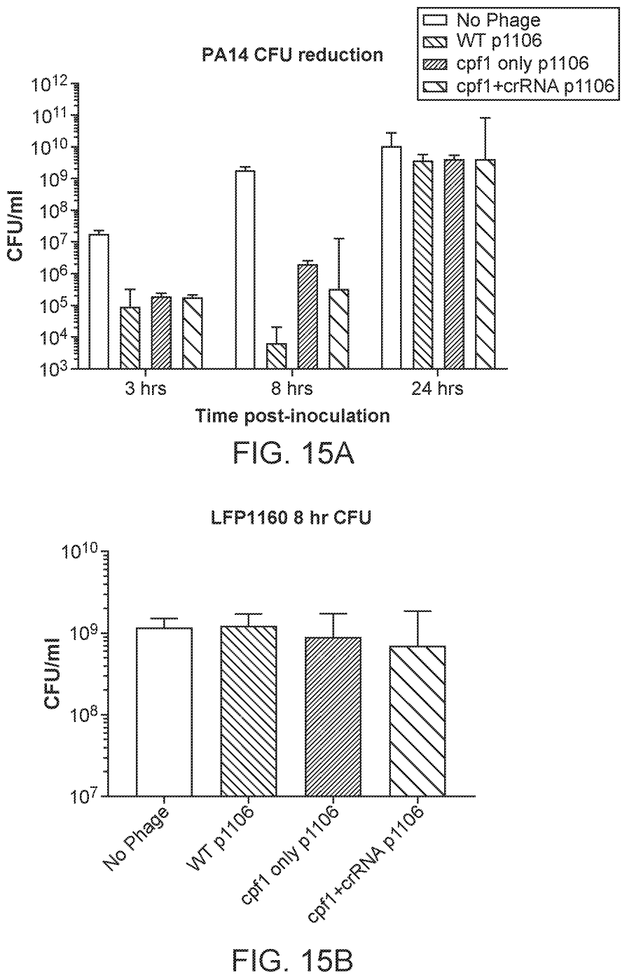

Phage p1106 and its engineered variants were incubated with a susceptible Pseudomonas aeruginosa strain (PA14) and a non-susceptible strain (LFP1160) and sampled at various times to enumerate bacterial CFUs. For PA14, the 3 hr reductions look similar for all 3 phages (˜2 log) (FIG. 15A). CFUs are equivalent for all groups in the non-susceptible strain (FIG. 15B).

### Example 14: Phage Gene Expression by Real-Time PCR

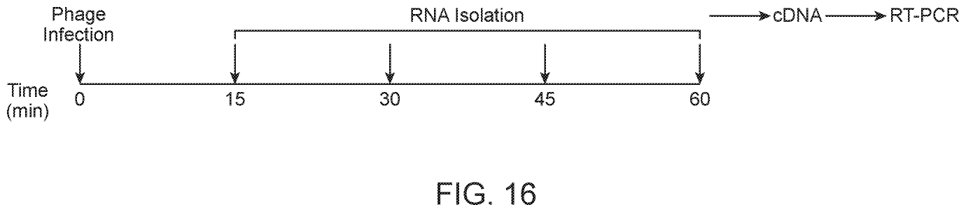

Studies were performed to determine the mRNA expression levels of Cpf1 and the bacterial genome targeting crRNA and to determine the level of ftsA cutting during bacteriophage infection. P. aeruginosa strain LFP1163 was utilized and two bacteriophages: wildtype p1032 (WT) and p1032_cpf1_full construct (crPhage). LFP1163 was infected with either WT bacteriophage or crPhage at a MOI=1. The study also included an uninfected LFP1163 control.) P. aeruginosa LFP1163/b1834 overnight culture back-diluted, grown to, and infected at OD=0.25). Study design is exemplified at FIG. 16.

Primers: rpsH (PA gene), Cpf1 (crPhage), crRNA (crPhage), DNA pol (all phage).

At 15 min. p.i., 30 min p.i., 45 min. p.i., and 60 min. p.i., bacterial cells were treated with RNAProtect, followed by bacterial cell lysis with Proteinase K and lysozyme. RNA was extracted with the Qiagen RNeasy Kit, with the on-column DNase treatment step. RNA concentration and quality (260/280 ratio) was determined by NanoDrop. RNA was converted into cDNA with the BioRad iSCRIPT cDNA synthesis protocol. mRNA levels of the targets were determined by real-time PCR with SYBR green: rpsH (bacterial housekeeping gene), Cpf1, crRNA (targeting the bacterial genome), uncut ftsA, cut ftsA, and phage DNA polymerase (phage infection positive control). Data were analyzed by the ΔΔCT method, using rpsH as the housekeeping control, followed by fold change calculations. This experiment was repeated twice. A subset of isolated RNA was sent to GeneWiz for RNA-Seq.

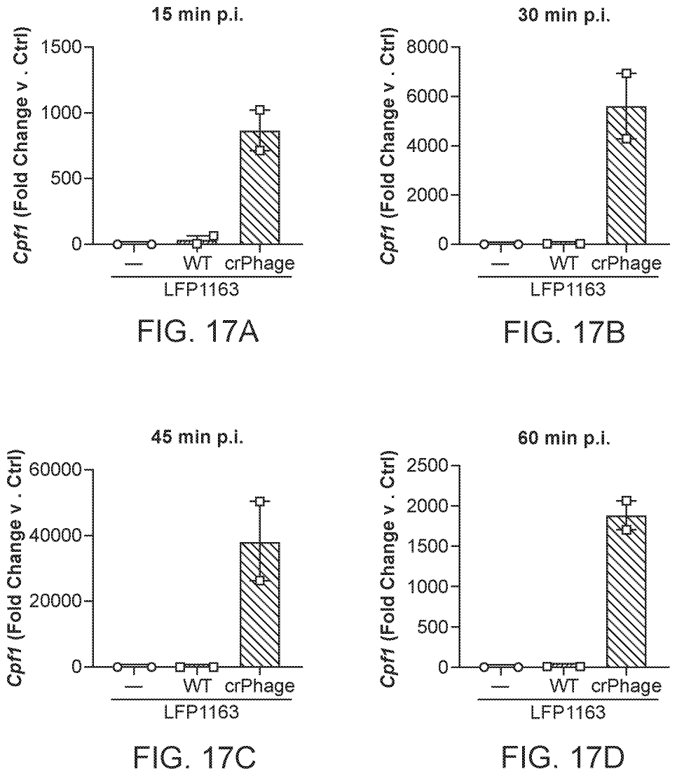

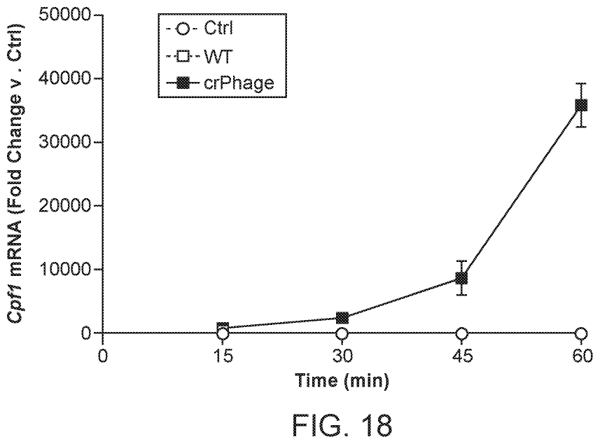

In FIG. 17A, FIG. 17B, FIG. 17C, FIG. 17D and FIG. 18 fold changes were derived by comparison to the uninfected control at each individual timepoint. The fold changes were compared against the P. aeruginosa housekeeping gene, rps. Background expression in the WT phage-infected bacteria was minimal. Cpf1 was expressed in the crPhage, validating the specificity of the primers for detecting CPFI expression,

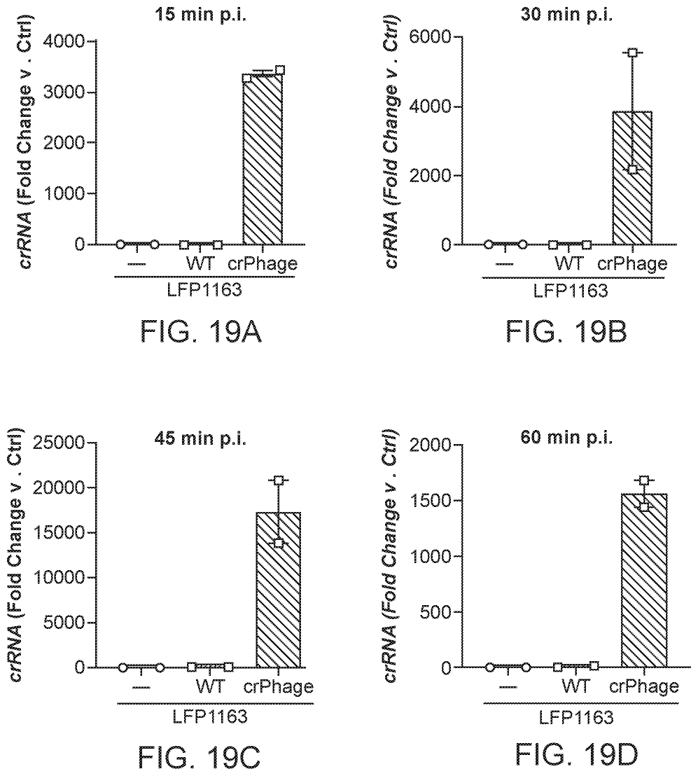

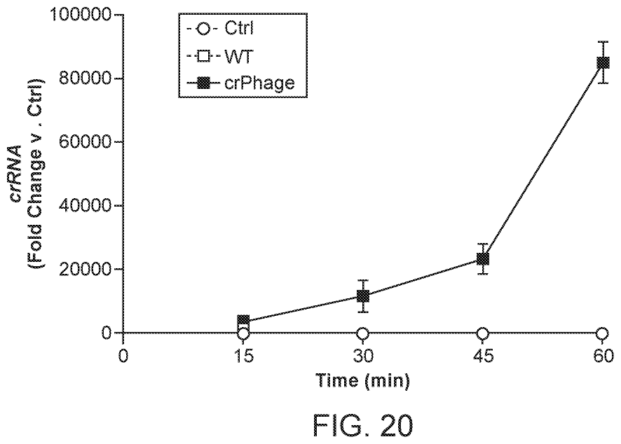

In FIG. 19A, FIG. 19B, FIG. 19C, FIG. 19D and FIG. 20 fold changes were derived by comparison to the uninfected control at each individual timepoint. Fold changes are compared against the P. aeruginosa housekeeping gene, rpsH. Background expression in the WT phage-infected bacteria was minimal. crRNA was expressed in the crPhage.

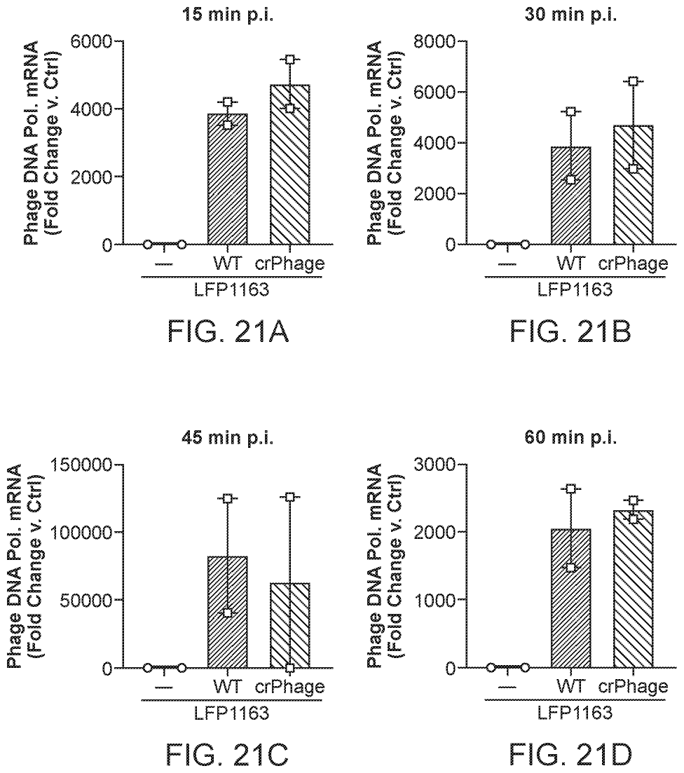

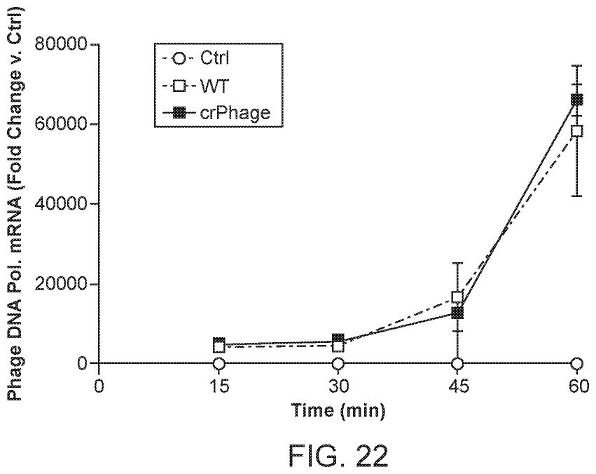

In FIG. 21A, FIG. 21B, FIG. 21C, FIG. 21D and FIG. 22 fold changes are derived by comparison to the uninfected control at each individual timepoint. Fold changes are compared against the P. aeruginosa housekeeping gene, rpsH. Phage DNA polymerase appears to be expressed in both WT phage and crPhage. In some cases, expression levels are very similar between WT and crPhage. The data shows that expression increased over time (FIG. 22).

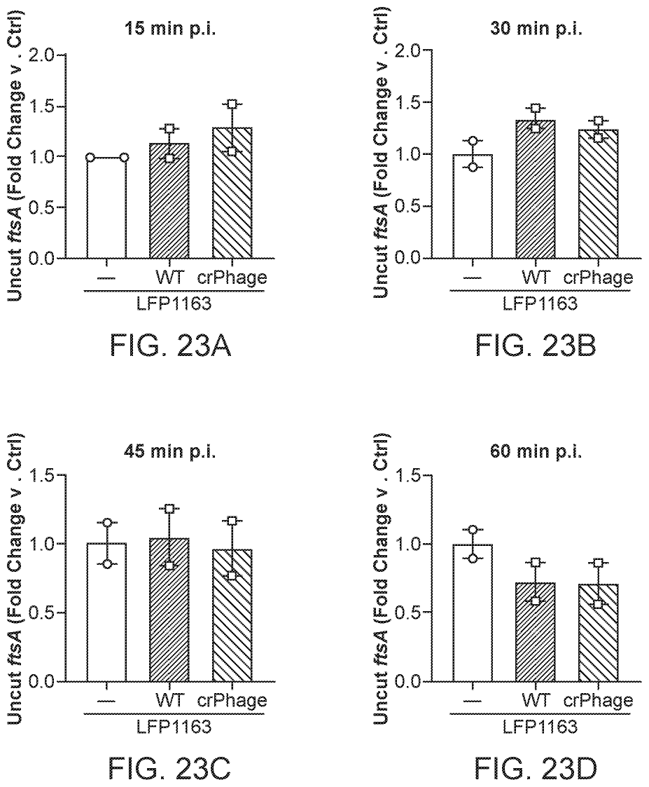

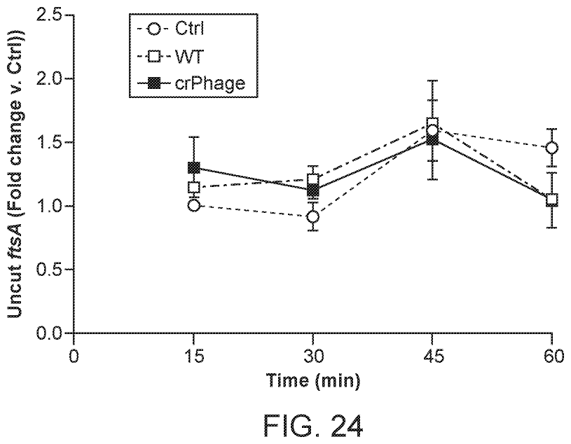

In FIG. 23A, FIG. 23B, FIG. 23C, FIG. 23D and FIG. 24 fold changes were derived by comparison to the uninfected control at each individual timepoint. Fold changes were compared against the P. aeruginosa housekeeping gene, rpsH. Uncut ftsA appears to be expressed at equal levels in all groups, until 60 min p.i. (FIG. 24).

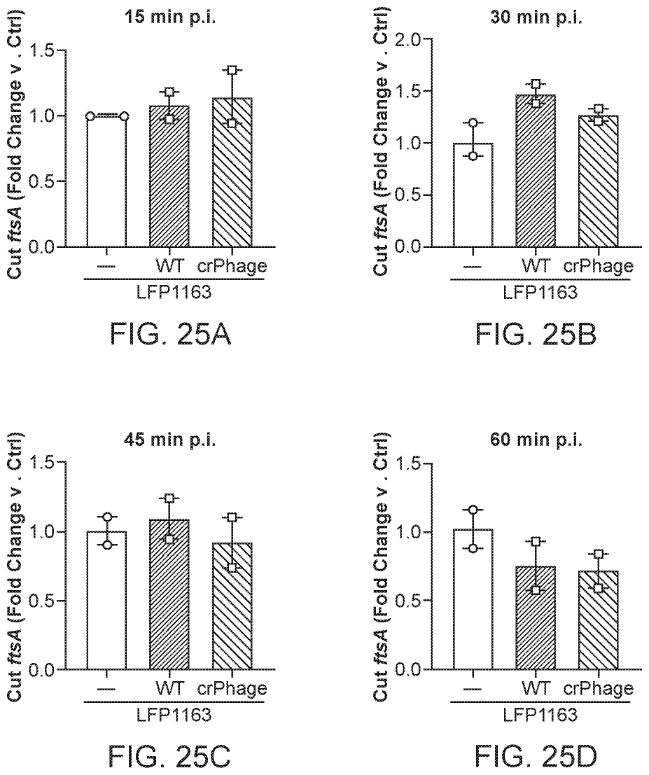

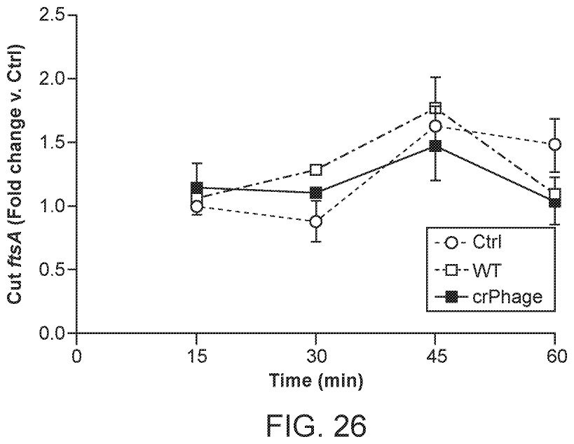

In FIG. 25A, FIG. 25B, FIG. 25C, FIG. 25D and FIG. 26 fold changes were derived by comparison to the uninfected control at each individual timepoint. Fold changes were compared against the P. aeruginosa housekeeping gene, rpsH. Cut ftsA were expressed at equal levels in all groups, until 60 min p.i. (FIG. 26).

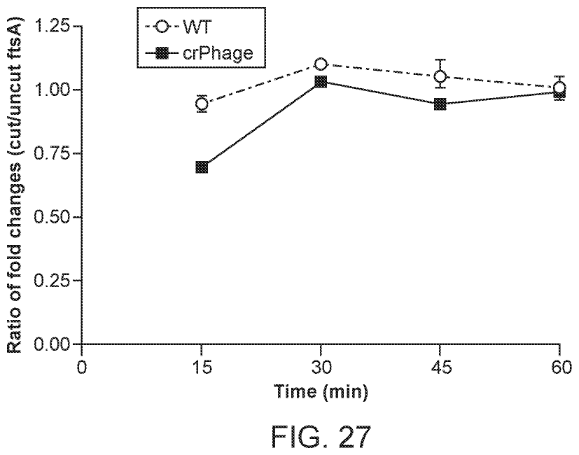

Ratio of cut/uncut ftsA by fold changes are shown in FIG. 27.

In some embodiments, in uninfected samples, there is no difference in the levels of “uncut” and “cut” expression of ftsA. In some embodiments, in WT phage infected samples, there is no difference in the levels of “uncut” and “cut” expression of ftsA. In some embodiments, in crPhage infected samples, there is a loss of the “cut” expression. In some embodiments, in crPhage infected sample a loss of “cut” expression is a result of loss of the DNA leading to the loss of mRNA. In some embodiments, a loss of “uncut” ftsA expression is due to loss of mRNA transcription/stability due to the downstream cutting of the DNA. In some embodiments, enhanced killing results in a reduced level of “uncut” ftsA. Loss of “uncut” ftsA in enhanced killing can be due to the level of bacterial death.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein are employed in practicing the disclosure. It is intended that the following claims define the scope of the disclosure and that methods and structures within the scope of these claims and their equivalents be covered thereby.

What is claimed is:

1. A method for killing a target bacterium comprising:
introducing into a target bacterium a bacteriophage comprising:
(a) a first nucleic acid encoding a spacer sequence or a crRNA transcribed therefrom, wherein the spacer sequence is complimentary to a target nucleotide sequence from a target gene in the target bacterium; and
(b) a second nucleic acid encoding a transcriptional activator for a CRISPR-Cpf1 system in a target bacterium;
wherein the target bacterium is killed by lytic activity of the bacteriophage or activity of a CRISPR-Cpf1 system using the spacer sequence or the crRNA transcribed therefrom.

2. The method of claim 1, wherein the first nucleic acid sequence is a CRISPR array further comprising at least one repeat sequence.

3. The method of any one of claims 1-2, wherein the transcriptional activator is endogenous to the target bacterium.

4. The method of any one of claims 1-2, wherein the transcriptional activator is exogenous to the target bacterium.

5. The method of any one of claims 2-4, wherein the transcriptional activator is regulated by Quorum Sensing (QS) signals.

6. The method of any one of claims 2-4, wherein the transcriptional activator is a protein involved in sensing stress of a bacterium membrane.

7. The method of any one of claims 2-4, wherein the transcriptional activator is a protein that stabilizes Cpf1.

8. The method of claim any one of claims 2-4, wherein the transcriptional activator is a metabolic sensing protein.

9. The method of claim 8, wherein the metabolic sensing protein is a sigma factor.

10. The method of any one of claims 2-4, wherein the transcriptional activator disrupts the activity of an inhibitory element.

11. The method of claim 10, wherein the inhibitory element is a transcriptional repressor.

12. The method of claim 11, wherein the transcriptional repressor is a global transcriptional repressor.

13. The method of any one of claims 1-12, wherein the CRISPR-Cpf1 system is endogenous to the target bacterium.

14. The method of any one of claims 1-12, wherein the CRISPR-Cpf1 system is exogenous to the target bacterium.

15. The method of any one of claims 1-14, wherein the target nucleotide sequence comprises all or a part of a promoter sequence for the target gene.

16. The method of any one of claims 1-15, wherein the target nucleotide sequence comprises all or a part of a nucleotide sequence located on a coding strand of a transcribed region of the target gene.

17. The method of any one of claims 1-16, wherein the target nucleotide sequence is at least a portion of an essential gene that is needed for the survival of the target bacterium.

18. The method of claim 17, wherein the essential gene is yfaP, speA, ftsZ, Tsf, acpP, gapA, infA, secY, csrA, trmD, ftsA, fusA, glyQ, eno, or nusG.

19. The method of any one of claims 2-18, wherein the at least one repeat sequence is operably linked to the at least one spacer sequence at either its 5′ end or its 3′ end.

20. The method of any one of claims 1-19, wherein the target bacterium is killed solely by the lytic activity of the bacteriophage.

21. The method of any one claims of 1-19, wherein the target bacterium is killed solely by the activity of the CRISPR-Cpf1 system.

22. The method of any of claims 1-19, wherein the target bacterium is killed by both the lytic activity of the bacteriophage and the activity of the CRISPR-Cpf1 system in combination.

23. The method of any of claims 1-19, wherein the target bacterium is killed by the activity of the CRISPR-Cpf1 system independently of the lytic activity of the bacteriophage.

24. The method of claim 22, wherein the activity of the CRISPR-Cpf1 system supplements or enhances the lytic activity of the bacteriophage.

25. The method of any one of claims 1-24, wherein the spacer nucleotide sequence overlaps with a second spacer sequence.

26. The method of any one of claims 1-25, wherein the lytic activity of the bacteriophage and the activity of the CRISPR-Cpf1 system are synergistic.

27. The method of any one of claims 1-26, wherein the lytic activity of the bacteriophage, the activity of the CRISPR-Cpf1 system, or both is modulated by a concentration of the bacteriophage.

28. The method of any one of claims 1-27, wherein the bacteriophage infects multiple bacterial strains.

29. The method of any one of claims 1-28, wherein the bacteriophage is an obligate lytic bacteriophage.

30. The method of any one of claims 1-28, wherein the bacteriophage is a temperate bacteriophage that is rendered lytic.

31. The method of claims 1-30, wherein the bacteriophage does not confer any new properties onto the target bacterium beyond cellular death caused by the lytic activity of the bacteriophage and/or the activity of the CRISPR-Cpf1 array.

32. The method of any one of claims 1-31, wherein the target bacterium is Escherichia coli, Klebsiella pneumoniae, Salmonella enterica, or Shigella dysenteriae.

33. The method of any one of claims 1-32, wherein the first nucleic acid encoding a spacer sequence or a crRNA is inserted into a non-essential bacteriophage gene.

34. The method of claim 33, wherein the non-essential bacteriophage gene is gp49.

35. The method of claim 33, wherein the non-essential bacteriophage gene is gp75.

36. The method of claim 33, wherein the non-essential bacteriophage gene is hoc.

37. The method of claim 33, wherein the non-essential bacteriophage gene is gp0.7, gp4.3, gp4.5, or gp4.7.

38. The method of claim 33, wherein the non-essential bacteriophage gene is gp0.6, gp0.65, gp0.7, gp4.3, or gp4.5.

39. The method of any one of claims 1-38, wherein the bacteriophage further comprises a third nucleic acid encoding a Gam protein.

40. A bacteriophage comprising:
(a) a first nucleic acid encoding a spacer sequence or a crRNA transcribed therefrom, wherein the spacer sequence is complimentary to a target nucleotide sequence from a target gene in a target bacterium; and
(b) a second nucleic acid encoding a encoding a transcriptional activator for a CRISPR-Cpf1 system in a target bacterium,
wherein the target bacterium is killed by the lytic activity of the bacteriophage or activity of a CRISPR-Cpf1 system using the spacer sequence or the crRNA transcribed therefrom.

41. The bacteriophage of claim 40, wherein the transcriptional activator is regulated by Quorum Sensing (QS) signals.

42. The bacteriophage of claim 40, wherein the transcriptional activator is a protein involved in sensing stress of a bacterium membrane.

43. The bacteriophage of claim 40, wherein the transcriptional activator is a protein that stabilizes Cpf1.

44. The bacteriophage of claim 40, wherein the transcriptional activator is a metabolic sensing protein.

45. The bacteriophage of claim 44, wherein the metabolic sensing protein is a sigma factor.

46. The bacteriophage of claim 40, wherein the transcriptional activator disrupts the activity of an inhibitory element of the target bacterium.

47. The bacteriophage of claim 46, wherein the inhibitory element is a transcriptional repressor.

48. The bacteriophage of claim 47, wherein the transcriptional repressor is a global transcriptional repressor.

49. The bacteriophage of any one of claims 40-48, wherein the CRISPR-Cpf1 system is endogenous to the target bacterium.

50. The bacteriophage of any one of claims 40-48, wherein the CRISPR-Cpf1 system is exogenous to the target bacterium.

51. The bacteriophage of any one of claims 40-50, wherein the target nucleotide sequence comprises all or a part of a promoter sequence for the target gene.

52. The bacteriophage of any one of claims 40-51, wherein the target nucleotide sequence comprises all or a part of a nucleotide sequence located on a coding strand of a transcribed region of the target gene.

53. The bacteriophage of any one of claims 40-52, wherein the target nucleotide sequence is essential.

54. The bacteriophage of claim 53, wherein the essential gene is yfaP, speA, ftsZ, Tsf, acpP, gapA, infA, secY, csrA, trmD, ftsA, fusA, glyQ, eno, or nusG.

55. The bacteriophage of any one of claims 40-52, wherein the target nucleotide sequence is a non-essential gene.

56. The bacteriophage of any one of claims 40-55, wherein the first nucleic acid sequence is a CRISPR array comprising at least one repeat sequence.

57. The bacteriophage of claim 56, wherein the at least one repeat sequence is operably linked to the spacer sequence at either its 5′ end or its 3′ end.

58. The bacteriophage of any one of claims 40-57, wherein the bacteriophage infects multiple bacterial strains.

59. The bacteriophage of any one of claims 40-58, wherein the bacteriophage is an obligate lytic bacteriophage.

60. The bacteriophage of any one of claims 40-58, wherein the bacteriophage is a temperate bacteriophage that is rendered lytic.

61. The bacteriophage of claim 60, wherein the temperate bacteriophage is rendered lytic by the removal, replacement, or inactivation of one or more lysogeny genes.

62. The bacteriophage of any one of claims 40-61, wherein the target bacterium is Escherichia coli, Klebsiella pneumoniae, Salmonella enterica, or Shigella dysenteriae.

63. The bacteriophage of any one of claims 40-62, wherein the first nucleic acid encoding a spacer sequence or a crRNA is inserted into a non-essential bacteriophage gene.

64. The bacteriophage of any one of claims 40-63, wherein the non-essential bacteriophage gene is gp49.

65. The bacteriophage of any one of claims 40-63, wherein the non-essential bacteriophage gene is gp75.

66. The bacteriophage of any one of claims 40-63, wherein the non-essential bacteriophage gene is hoc.

67. The bacteriophage of any one of claims 40-63, wherein the non-essential bacteriophage gene is gp0.7, gp4.3, gp4.5, or gp4.7.

68. The bacteriophage of any one of claims 40-63, wherein the non-essential bacteriophage gene is gp0.6, gp0.65, gp0.7, gp4.3, or gp4.5.

69. The bacteriophage of any one of claims 40-68, further comprising a third nucleic acid encoding a Gam protein.

70. A method for modulating the activity of a CRISPR-Cpf1 system in a bacterium, comprising:
introducing a bacteriophage comprising a nucleic acid encoding a transcriptional activator for the CRISPR-Cpf1 system in the target bacterium.

71. The method of claim 70, wherein the transcriptional activator is regulated by Quorum Sensing (QS) signals.

72. The method of claim 70, wherein the transcriptional activator is a protein involved in sensing stress to a bacterium membrane.

73. The method of claim 70, wherein the transcriptional activator is a protein that stabilizes Cpf1.

74. The method of claim 70, wherein the transcriptional activator is a metabolic sensing protein.

75. The method of claim 74, wherein the metabolic sensing protein is a sigma factor.

76. The method of any one of claims 70-75, wherein the transcriptional activator disrupts the activity of an inhibitory element.

77. The method of claim 76, wherein the inhibitory element is a transcriptional repressor.

78. The method of claim 77, wherein the transcriptional repressor is a global transcriptional repressor.

79. The method of any one of claims 70-78, wherein the CRISPR-Cpf1 system is endogenous to the target bacterium.

80. The method of any one of claims 70-78, wherein the CRISPR-Cpf1 system is exogenous to the target bacterium.

81. The method of any one of claims 70-80, wherein the bacteriophage infects multiple bacterial strains.

82. The method of any one of claims 70-81, wherein the bacteriophage is an obligate lytic bacteriophage.

83. The method of any one of claims 70-82, wherein the bacteriophage is a temperate bacteriophage that is rendered lytic.

84. The method of any one of claims 70-83, wherein the target bacterium is Escherichia coli, Klebsiella pneumoniae, Salmonella enterica, or Shigella dysenteriae.

85. The method of any one of claims 70-84, wherein the nucleic acid encoding a transcriptional activator is inserted into a non-essential bacteriophage gene.

86. The method of any one of claims 70-85 wherein the non-essential bacteriophage gene is gp49.

87. The method of any one of claims 70-85, wherein the non-essential bacteriophage gene is gp75.

88. The method of any one of claims 70-85, wherein the non-essential bacteriophage gene is hoc.

89. The method of any one of claims 70-85, wherein the non-essential bacteriophage gene is gp0.7, gp4.3, gp4.5, or gp4.7.

90. The method of any one of claims 70-85, wherein the non-essential bacteriophage gene is gp0.6, 0.65, 0.7, 4.3, or gp4.5.

91. The method of any one of claims 70-90, wherein the bacteriophage further comprises a second nucleic acid encoding a Gam protein.

92. A bacteriophage comprising a nucleic acid encoding a transcriptional activator for a CRISPR-Cpf1 system in a target bacterium.

93. The bacteriophage of claim 92, wherein the transcriptional activator is regulated by Quorum Sensing (QS) signals.

94. The bacteriophage of claim 92, wherein the transcriptional activator is a protein involved in sensing stress to a bacterium membrane.

95. The bacteriophage of claim 92, wherein the transcriptional activator is a protein that stabilizes Cpf1.

96. The bacteriophage of claim 92, wherein the transcriptional activator is a metabolic sensing protein.

97. The bacteriophage of claim 93, wherein the metabolic sensing protein is a sigma factor.

98. The bacteriophage of any one of claims 92-97, wherein the transcriptional activator disrupts the activity of an inhibitory element.

99. The bacteriophage of claim 98, wherein the inhibitory element is a transcriptional repressor.

100. The bacteriophage of claim 99, wherein the transcriptional repressor is a global transcriptional repressor.

101. The bacteriophage of any one of claims 92-100, wherein the CRISPR-Cpf1 system is endogenous to the target bacterium.

102. The bacteriophage of any one of claims 92-100, wherein the CRISPR-Cpf1 system is exogenous to the target bacterium.

103. The bacteriophage of any one of claims 92-102, wherein the bacteriophage infects multiple bacterial strains.

104. The bacteriophage of any one of claims 92-103, wherein the bacteriophage is an obligate lytic bacteriophage.

105. The bacteriophage of any one of claims 92-103, wherein the bacteriophage is a temperate bacteriophage that is rendered lytic.

106. The bacteriophage of any one of claims 92-105, wherein the target bacterium is Escherichia coli, Klebsiella pneumoniae, Salmonella enterica, or Shigella dysenteriae.

107. The bacteriophage of any one of claims 92-106, wherein the nucleic acid encoding a transcriptional activator is inserted into a non-essential bacteriophage gene.

108. The bacteriophage of any one of claims 92-107, wherein the non-essential gene is gp49.

109. The bacteriophage of any one of claims 92-107, wherein the non-essential gene is gp75.

110. The bacteriophage of any one of claims 92-107, wherein the non-essential gene is hoc.

111. The bacteriophage of any one of claims 39-107, wherein the non-essential gene is gp0.7, 4.3, 4.5, or gp4.7.

112. The bacteriophage of any one of claims 39-107, wherein the non-essential gene is gp0.6, 0.65, 0.7, 4.3, or gp4.5.

113. The bacteriophage of any one of claims 92-112, further comprising a second nucleic acid encoding a Gam protein.

114. A method of killing a target bacterium, comprising introducing into a target bacterium a bacteriophage comprising:
(a) lytic activity, and
(b) a first nucleic acid sequence encoding an anti-CRISPR polypeptide,
wherein the anti-CRISPR polypeptide enhances the lytic activity of the bacteriophage.

115. The method of claim 114, wherein the anti-CRISPR polypeptide inactivates a CRISPR-Cpf1 system.

116. The method of claim 115, wherein the anti-CRISPR polypeptide inactivates the CRISPR-Cpf1 system using a process comprising gene regulation interference.

117. The method of any one of claims 115-116, wherein the anti-CRISPR polypeptide inactivates the CRISPR-Cpf1 system using a process comprising nuclease recruitment interference.

118. The method of any one of claims 114-117, wherein the anti-CRISPR polypeptide is a truncated protein, a fusion protein, a dimer protein or mutated protein.

119. The method of any one of claims 114-118, wherein the bacteriophage further comprises a second nucleic acid encoding a CRISPR array.

120. The method of claim 119, wherein the CRISPR array comprises at least one repeat sequence and at least one spacer sequence that is complimentary to a target nucleotide sequence from a target gene in the target bacterium.

121. A bacteriophage comprising:
(a) lytic activity, and
(b) a first nucleic acid sequence encoding an anti-CRISPR polypeptide,
wherein the anti-CRISPR polypeptide enhances the lytic activity of the bacteriophage.

122. The bacteriophage of claim 121, wherein the anti-CRISPR polypeptide inactivates a CRISPR-Cpf1 system.

123. The bacteriophage of claim 122, wherein the anti-CRISPR polypeptide inactivates the CRISPR-Cpf1 system using a process comprising gene regulation interference.

124. The bacteriophage of claim 112 or 123, wherein the anti-CRISPR polypeptide inactivates the CRISPR-Cpf1 system using a process comprising nuclease recruitment interference.

125. The bacteriophage of any one of claims 121-124, wherein the anti-CRISPR polypeptide is a truncated protein, a fusion protein, a dimer protein or mutated protein.

126. The bacteriophage of any one of claims 121-124, wherein the bacteriophage further comprises a second nucleic acid encoding a CRISPR array.

127. The bacteriophage of claim 126, wherein the CRISPR array comprises at least one repeat sequence and at least one spacer sequence that is complimentary to a target nucleotide sequence from a target gene in the target bacterium.

128. A method of treating a disease in a subject comprising administering the bacteriophage of any one of claim 40-69, 92-113 or 121-127 to the subject.

129. The method of claim 128, wherein the subject is a mammal.

130. The method of any one of claims 128-129, wherein the disease is a bacterial infection.

131. The method of claim 130, wherein a bacterium causing the bacterial infection is a bacterium in the genus Acinetobacter, Actinomyces, Burkholderia, Capylobacter, Candidia, Clostrium, Corynebacterium, Coccidiodes, Cryptococcus, Enterococcus, Escherichica, Haemophilis, Helicobacter, Klebsiella, Moraxella, Mycobacterium, Neisseria, Porphyromonas, Prevotella, Pseudomonas, Salmonella, Serratia, Staphylococcus, Streptococcus, or Vibrio.

132. The method of claim 130, wherein a bacterium causing the bacterial infection is Burkholderia cepacia, Clostridium difficile, Corynebacterium minutissium, Corynebacterium pseudodiphtherias, Corynebacterium stratium, Escherichia coli, Haemophilus influenzae, Klebsiella pneumoniae, a Moraxella species, Mycobacterium tuberculosis, Neisseria gonorrhoeae, Neisseria meningitidis, Prevotella melaninogenicus, Salmonella typhimurium, Salmonella enterica, Shigella dysenteriae, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, Helicobacter felis, Helicobacter pylori, or Clostridium bolteae.

133. The method of any one of claim 131 or 132, wherein the bacterium is a drug resistant bacteria that is resistant to at least one antibiotic.

134. The method of any one of claims 131-133, wherein the bacterium is a multi-drug resistant bacteria that is resistant to at least one antibiotic.

135. The method of any one of claim 133 or 134, wherein the antibiotic comprises a cephalosporin, a fluoroquinolone, a carbapenem, a colistin, an aminoglycoside, vancomycin, streptomycin, or methicillin.

136. The method of any one of claims 128-135, wherein the administering is intra-arterial, intravenous, intramuscular, oral, subcutaneous, inhalation, or any combination thereof.

137. A pharmaceutical composition comprising:
a. a bacteriophage of any one of claim 40-69, 92-113 or 121-127; and
b. a pharmaceutically acceptable excipient.

138. The pharmaceutical composition of claim 137, that is in a form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof.

139. A method for killing a target bacterium comprising:
introducing into a target bacterium a bacteriophage comprising:
(a) a first nucleic acid encoding a spacer sequence or a crRNA transcribed therefrom, wherein the spacer sequence is complimentary to a target nucleotide sequence from a target gene in the target bacterium; and
(b) a second nucleic acid encoding an exogenous Cpf1;
wherein the target bacterium is killed by lytic activity of the bacteriophage or activity of a CRISPR-Cpf1 system using the spacer sequence or the crRNA transcribed therefrom and the exogenous Cpf1.

140. The method of claim 139, wherein the first nucleic acid sequence is a CRISPR array further comprising at least one repeat sequence.

141. The method of any one of claims 139-140, wherein the CRISPR-Cpf1 system is endogenous to the target bacterium.

142. The method of any one of claims 139-140, wherein the CRISPR-Cpf1 system is exogenous to the target bacterium.

143. The method of any one of claims 139-142, wherein the target nucleotide sequence comprises all or a part of a promoter sequence for the target gene.

144. The method of any one of claims 139-143, wherein the target nucleotide sequence comprises all or a part of a nucleotide sequence located on a coding strand of a transcribed region of the target gene.

145. The method of any one of claims 139-144, wherein the target nucleotide sequence is at least a portion of an essential gene that is needed for the survival of the target bacterium.

146. The method of claim 145, wherein the essential gene is yfaP, speA, ftsZ, Tsf, acpP, gapA, infA, secY, csrA, trmD, ftsA, fusA, glyQ, eno, or nusG.

147. The method of any one of claims 140-146, wherein the at least one repeat sequence is operably linked to the at least one spacer sequence at either its 5′ end or its 3′ end.

148. The method of any one of claims 139-147, wherein the target bacterium is killed solely by the lytic activity of the bacteriophage.

149. The method of any one claims of 139-147, wherein the target bacterium is killed solely by the activity of the CRISPR-Cpf1 system.

150. The method of any of claims 139-147, wherein the target bacterium is killed by both the lytic activity of the bacteriophage and the activity of the CRISPR-Cpf1 system in combination.

151. The method of any of claims 139-147, wherein the target bacterium is killed by the activity of the CRISPR-Cpf1 system independently of the lytic activity of the bacteriophage.

152. The method of claim 151, wherein the activity of the CRISPR-Cpf1 system supplements or enhances the lytic activity of the bacteriophage.

153. The method of any one of claims 139-152, wherein the spacer nucleotide sequence overlaps with a second spacer sequence.

154. The method of any one of claims 139-153, wherein the lytic activity of the bacteriophage and the activity of the CRISPR-Cpf1 system are synergistic.

155. The method of any one of claims 139-154, wherein the lytic activity of the bacteriophage, the activity of the CRISPR-Cpf1 system, or both is modulated by a concentration of the bacteriophage.

156. The method of any one of claims 139-155, wherein the bacteriophage infects multiple bacterial strains.

157. The method of any one of claims 139-156, wherein the bacteriophage is an obligate lytic bacteriophage.

158. The method of any one of claims 139-156, wherein the bacteriophage is a temperate bacteriophage that is rendered lytic.

159. The method of claims 139-158, wherein the bacteriophage does not confer any new properties onto the target bacterium beyond cellular death caused by the lytic activity of the bacteriophage and/or the activity of the CRISPR-Cpf1 array.

160. The method of any one of claims 139-159, wherein the target bacterium is Escherichia coli, Klebsiella pneumoniae, Salmonella enterica, or Shigella dysenteriae.

161. The method of any one of claims 139-160, wherein the first nucleic acid encoding a spacer sequence or a crRNA is inserted into a non-essential bacteriophage gene.

162. The method of claim 161, wherein the non-essential bacteriophage gene is gp49.

163. The method of claim 161, wherein the non-essential bacteriophage gene is gp75.

164. The method of claim 161, wherein the non-essential bacteriophage gene is hoc.

165. The method of claim 161, wherein the non-essential bacteriophage gene is gp0.7, gp4.3, gp4.5, or gp4.7.

166. The method of claim 161, wherein the non-essential bacteriophage gene is gp0.6, gp0.65, gp0.7, gp4.3, or gp4.5.

167. The method of any one of claims 139-166, wherein the bacteriophage further comprises a third nucleic acid encoding a Gam protein.

168. A bacteriophage comprising:
(a) a first nucleic acid encoding a spacer sequence or a crRNA transcribed therefrom, wherein the spacer sequence is complimentary to a target nucleotide sequence from a target gene in a target bacterium; and
(b) a second nucleic acid encoding a encoding an exogenous Cpf1,
wherein the target bacterium is killed by the lytic activity of the bacteriophage or activity of a CRISPR-Cpf1 system using the spacer sequence or the crRNA transcribed therefrom and the exogenous Cpf1.

169. The bacteriophage of claim 168, wherein the CRISPR-Cpf1 system is endogenous to the target bacterium.

170. The bacteriophage of claim 168, wherein the CRISPR-Cpf1 system is exogenous to the target bacterium.

171. The bacteriophage of any one of claims 168-170, wherein the target nucleotide sequence comprises all or a part of a promoter sequence for the target gene.

172. The bacteriophage of any one of claims 168-171, wherein the target nucleotide sequence comprises all or a part of a nucleotide sequence located on a coding strand of a transcribed region of the target gene.

173. The bacteriophage of any one of claims 168-172, wherein the target nucleotide sequence is essential.

174. The bacteriophage of claim 173, wherein the essential gene is yfaP, speA, ftsZ, Tsf, acpP, gapA, infA, secY, csrA, trmD, ftsA, fusA, glyQ, eno, or nusG.

175. The bacteriophage of any one of claims 168-172, wherein the target nucleotide sequence is a non-essential gene.

176. The bacteriophage of any one of claims 168-175, wherein the first nucleic acid sequence is a CRISPR array comprising at least one repeat sequence.

177. The bacteriophage of claim 176, wherein the at least one repeat sequence is operably linked to the spacer sequence at either its 5′ end or its 3′ end.

178. The bacteriophage of any one of claims 168-177, wherein the bacteriophage infects multiple bacterial strains.

179. The bacteriophage of any one of claims 168-178, wherein the bacteriophage is an obligate lytic bacteriophage.

180. The bacteriophage of any one of claims 168-178, wherein the bacteriophage is a temperate bacteriophage that is rendered lytic.

181. The bacteriophage of claim 180, wherein the temperate bacteriophage is rendered lytic by the removal, replacement, or inactivation of one or more lysogeny genes.

182. The bacteriophage of any one of claims 168-181, wherein the target bacterium is Escherichia coli, Klebsiella pneumoniae, Salmonella enterica, or Shigella dysenteriae.

183. The bacteriophage of any one of claims 168-182, wherein the first nucleic acid encoding a spacer sequence or a crRNA is inserted into a non-essential bacteriophage gene.

184. The bacteriophage of any one of claims 168-183, wherein the non-essential bacteriophage gene is gp49.

185. The bacteriophage of any one of claims 168-183, wherein the non-essential bacteriophage gene is gp75.

186. The bacteriophage of any one of claims 168-183, wherein the non-essential bacteriophage gene is hoc.

187. The bacteriophage of any one of claims 168-183, wherein the non-essential bacteriophage gene is gp0.7, gp4.3, gp4.5, or gp4.7.

188. The bacteriophage of any one of claims 168-183, wherein the non-essential bacteriophage gene is gp0.6, gp0.65, gp0.7, gp4.3, or gp4.5.

189. The bacteriophage of any one of claims 168-188, further comprising a third nucleic acid encoding a Gam protein.

190. A method for modulating the activity of a CRISPR-Cpf1 system in a bacterium, comprising:
introducing a bacteriophage comprising a nucleic acid encoding an exogenous Cpf1 for the CRISPR-Cpf1 system in the target bacterium.

191. The method of claim 190, wherein the CRISPR-Cpf1 system is endogenous to the target bacterium.

192. The method of claim 190, wherein the CRISPR-Cpf1 system is exogenous to the target bacterium.

193. The method of any one of claims 190-192, wherein the bacteriophage infects multiple bacterial strains.

194. The method of any one of claims 190-193, wherein the bacteriophage is an obligate lytic bacteriophage.

195. The method of any one of claims 190-193, wherein the bacteriophage is a temperate bacteriophage that is rendered lytic.

196. The method of any one of claims 190-195, wherein the target bacterium is Escherichia coli, Klebsiella pneumoniae, Salmonella enterica, or Shigella dysenteriae.

197. The method of any one of claims 190-196, wherein the bacteriophage further comprises a second nucleic acid encoding a Gam protein.

198. A bacteriophage comprising a nucleic acid encoding an exogenous Cpf1 for a CRISPR-Cpf1 system in a target bacterium.

199. The bacteriophage of claim 198, wherein the CRISPR-Cpf1 system is endogenous to the target bacterium.

200. The bacteriophage of claim 198, wherein the CRISPR-Cpf1 system is exogenous to the target bacterium.

201. The bacteriophage of any one of claims 198-200, wherein the bacteriophage infects multiple bacterial strains.

202. The bacteriophage of any one of claims 198-201, wherein the bacteriophage is an obligate lytic bacteriophage.

203. The bacteriophage of any one of claims 198-201, wherein the bacteriophage is a temperate bacteriophage that is rendered lytic.

204. The bacteriophage of any one of claims 198-203, wherein the target bacterium is Escherichia coli, Klebsiella pneumoniae, Salmonella enterica, or Shigella dysenteriae.

205. The bacteriophage of any one of claims 198-204, further comprising a second nucleic acid encoding a Gam protein.

206. A method of treating a disease in a subject comprising administering the bacteriophage of any one of claim 168-189, or 198-205 to the subject.

207. The method of claim 206, wherein the subject is a mammal.

208. The method of any one of claims 206-207, wherein the disease is a bacterial infection.

209. The method of claim 208, wherein a bacterium causing the bacterial infection is a bacterium in the genus Acinetobacter, Actinomyces, Burkholderia, Capylobacter, Candidia, Clostrium, Corynebacterium, Coccidiodes, Cryptococcus, Enterococcus, Escherichica, Haemophilis, Helicobacter, Klebsiella, Moraxella, Mycobacterium, Neisseria, Porphyromonas, Prevotella, Pseudomonas, Salmonella, Serratia, Staphylococcus, Streptococcus, or Vibrio.

210. The method of claim 208, wherein a bacterium causing the bacterial infection is Burkholderia cepacia, Clostridium difficile, Corynebacterium minutissium, Corynebacterium pseudodiphtheriae, Corynebacterium stratium, Escherichia coli, Haemophilus influenzae, Klebsiella pneumoniae, a Moraxella species, Mycobacterium tuberculosis, Neisseria gonorrhoeae, Neisseria meningitidis, Prevotella melaninogenicus, Salmonella typhimurium, Salmonella enterica, Shigella dysenteriae, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, Helicobacter felis, Helicobacter pylori, or Clostridium bolteae.

211. The method of any one of claim 209 or 210, wherein the bacterium is a drug resistant bacteria that is resistant to at least one antibiotic.

212. The method of any one of claims 209-211, wherein the bacterium is a multi-drug resistant bacteria that is resistant to at least one antibiotic.

213. The method of any one of claim 211 or 212, wherein the antibiotic comprises a cephalosporin, a fluoroquinolone, a carbapenem, a colistin, an aminoglycoside, vancomycin, streptomycin, or methicillin.

214. The method of any one of claims 206-213, wherein the administering is intra-arterial, intravenous, intramuscular, oral, subcutaneous, inhalation, or any combination thereof.

215. A pharmaceutical composition comprising:
a. a bacteriophage of any one of claim 168-189, or 198-205; and
b. a pharmaceutically acceptable excipient.

216. The pharmaceutical composition of claim 215, that is in a form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof.
